SEQUENCE LISTING (79 sequences)

SEQ ID NO: 1 - protein, 451 aa, synthetic construct
QLQVQESGPGLVKPSETLSLTCTVSGGSISNSYFSWGWIRQPPGKGLEWIGSFYYGEKTYYNPSLKSRATISIDTSKSQFSLKLSSVTAADTAVYYCPRGPTMIRGVIDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 214 aa, synthetic construct
EIVLTQSPATLSLSPGERATLSCRASQSVRSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 454 aa, synthetic construct
EVQLVESGGGVVQPGRSLRLSCAASGFTFSNYGMHWVRQAPGKGLEWVAVISYDGSTKYSADSLKGRFTISRDNSKNTLYLQMNSLRADDTAVYYCAKEGWSFDSSGYRSWFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 4 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 454 aa, synthetic construct
EVQLVESGGGVVQPGRSLRLSCAASGFTFSNYGMHWVRQAPGKGLEWVAVISYDGSTKYSADSLKGRFTISRDNSKNTLYLQMNSLRADDTAVYYCAKEGWSFDSSGYRSWFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELGRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - protein, 760 aa, Homo sapiens
MKTWVKIVFGVATSAVLALLVMCIVLRPSRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPVVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEEYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSD

SEQ ID NO: 7 - protein, 454 aa, synthetic construct
QVQLVQSGAEVKKPGASVKVSCKTSRYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 220 aa, synthetic construct
DIVMTQSPDSLAVSLGERATINCKSSQSLLYSRNQKNYLAWYQQKPGQPPKLLIFWASTRESGVPDRFSGSGFGTDFTLTISSLQAEDVAVYYCQQYFSYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 760 aa, unidentified
  source 1..760: note = Cynomolgus macaque
MKTWVKIVFGVATSAVLALLVMCIVLRPPRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPFVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEDYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSD

SEQ ID NO: 10 - protein, 766 aa, Homo sapiens
MKTPWKVLLGLLGAAALVTIITVPVVLLNKGTDDATADSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWSYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLP

SEQ ID NO: 11 - protein, 121 aa, synthetic construct
EVQLVESGGGLVQPGGSLRLSCAASGFTFSSYAMIWVRQAPGKGLEWVSAISGSGGYTFNADSVKGRFTMSRDNSKNTLYLQMNSLRAEDTAVYYCAKRDGGYEGGAFDIWGQGTLVTVSS

SEQ ID NO: 12 - protein, 5 aa, synthetic construct
SYAMI

SEQ ID NO: 13 - protein, 17 aa, synthetic construct
AISGSGGYTFNADSVKG

SEQ ID NO: 14 - protein, 12 aa, synthetic construct
RDGGYEGGAFDI

SEQ ID NO: 15 - protein, 123 aa, synthetic construct
QLQLQESGPGLVKPSETLSLTCTVSGGSISSSSYFWAWIRQPPGKGLEFIGNIYYSGSTYYNPSLKSRVTISVDTSKNQFSLKLSSVTAADTAVYYCARPSYYGSGNYYFAFWGQGTLVTVSS

SEQ ID NO: 16 - protein, 7 aa, synthetic construct
SSSYFWA

SEQ ID NO: 17 - protein, 16 aa, synthetic construct
NIYYSGSTYYNPSLKS

SEQ ID NO: 18 - protein, 13 aa, synthetic construct
PSYYGSGNYYFAF

SEQ ID NO: 19 - protein, 122 aa, synthetic construct
EVQLVESGGGLVKPGGSLRLSCAASGFPFNYAWMSWVRQAPGKGLEWVGRIKPKTSGGATDYAAPVKDRFTISRDDSRNTLYLQMNSLKTEDTAVYYCSAREDIWNFFFDLWGQGTLVTVSS

SEQ ID NO: 20 - protein, 5 aa, synthetic construct
YAWMS

SEQ ID NO: 21 - protein, 19 aa, synthetic construct
RIKPKTSGGATDYAAPVKD

SEQ ID NO: 22 - protein, 11 aa, synthetic construct
REDIWNFFFDL

SEQ ID NO: 23 - protein, 121 aa, synthetic construct
QVQLVESGGGLVQPGGSLRLSCAVSGFTFRRYAMSWVRQAPGKGLEWVSAISASGDRTHNTDSVKGRFSISRDNSKNTLYLQMNSLRAEDTAVYFCAKGIAASGKNYFDPWGQGTLVTVSS

SEQ ID NO: 24 - protein, 5 aa, synthetic construct
RYAMS

SEQ ID NO: 25 - protein, 17 aa, synthetic construct
AISASGDRTHNTDSVKG

SEQ ID NO: 26 - protein, 12 aa, synthetic construct
GIAASGKNYFDP

SEQ ID NO: 27 - protein, 121 aa, synthetic construct
QVQLVESGGGLVQPGGSLRLSCAVSGFTFSRYAMSWVRQAPGKGLEWVSAISASGDRTKNTDSVKGRFSISRDNSKNTLYLQMNSLRAEDTAVYFCAKGTAAAGKNYFDPWGQGTLVTVSS

SEQ ID NO: 28 - protein, 96 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSP

SEQ ID NO: 29 - protein, 17 aa, synthetic construct
AISASGDRTKNTDSVKG

SEQ ID NO: 30 - protein, 12 aa, synthetic construct
GTAAAGKNYFDP

SEQ ID NO: 31 - protein, 121 aa, synthetic construct
QVQLVESGGGLVQPGGSLRLSCAVSGFTFERYAMSWVRQAPGKGLEWVSAISASGDRTQNTDSVKGRFSISRDNSKNTLYLQMNSLRAEDTAVYFCAKGTAASGRNYFDPWGQGTLVTVSS

SEQ ID NO: 32 - protein, 108 aa, synthetic construct
SYVLTQPPSVSVAPGETARITCGGDNIGRKSVYWYQQKSGQAPVLVIYYDSDRPSGIPERFSGSNSGNTATLTISRVEAGDEADYYCQVWDGSSDHWVFGGGTKLTVL

SEQ ID NO: 33 - protein, 17 aa, synthetic construct
AISASGDRTQNTDSVKG

SEQ ID NO: 34 - protein, 12 aa, synthetic construct
GTAASGRNYFDP

SEQ ID NO: 35 - protein, 121 aa, synthetic construct
QVQLVESGGGLVQPGGSLRLSCAVSGFTFERYAMSWVRQAPGKGLEWVSAISASGDRTQYTDSVKGRFSISRDNSKNTLYLQMNSLRAEDTAVYFCAKGTAASGRNYFDPWGQGTLVTVSS

SEQ ID NO: 36 - protein, 6 aa, synthetic construct
NIGRKS

SEQ ID NO: 37 - protein, 17 aa, synthetic construct
AISASGDRTQYTDSVKG

SEQ ID NO: 38 - skipped

SEQ ID NO: 39 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 40 - protein, 15 aa, synthetic construct
EPKSCDKTHTCPPCP

SEQ ID NO: 41 - protein, 110 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 42 - protein, 110 aa, synthetic construct
APELGRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 43 - protein, 106 aa, synthetic construct
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 44 - protein, 106 aa, synthetic construct
GQPREPQVYTDPPSREEMTKNQVSLTCEVKGFYPSDIAVEWESNGQPENNYKTTPPVLDSDGSFFLYSKLTVDKSRWQQGNVFSCSVMHEALHNHYTQKSLSLSPG

SEQ ID NO: 45 - protein, 106 aa, synthetic construct
GQPREPQVYTKPPSREEMTKNQVSLKCLVKGFYPSDIAVEWESNGQPENNYKTTPPVLDSDGSFFLYSKLTVDKSRWQQGNVFSCSVMHEALHNHYTQKSLSLSPG

SEQ ID NO: 46 - protein, 567 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 47 - protein, 144 aa, Homo sapiens
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQ

SEQ ID NO: 48 - protein, 592 aa, Homo sapiens
MGRGLLRGLWPLHIVLWTRIASTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 49 - protein, 169 aa, Homo sapiens
TIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQ

SEQ ID NO: 50 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 51 - protein, 107 aa, synthetic construct [sequence omitted]

SEQ ID NO: 52 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIK

SEQ ID NO: 53 - protein, 6 aa, synthetic construct
QSISSY

SEQ ID NO: 54 - skipped

SEQ ID NO: 55 - protein, 9 aa, synthetic construct
QQSYSTPPT

SEQ ID NO: 56 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 57 - protein, 11 aa, synthetic construct
QVWDGSSDHWV

SEQ ID NO: 58 - protein, 96 aa, synthetic construct
SYVLTQPPSVSVAPGETARITCGGDNIGRKSVYWYQQKSGQAPVLVIYYDSDRPSGIPERFSGSNSGNTATLTISRVEAGDEADYYCQVWDGSSDH

SEQ ID NO: 59 - protein, 10 aa, synthetic construct
QQSYSTPPIT

SEQ ID NO: 60 - protein, 95 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTP

SEQ ID NO: 61 - protein, 107 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPWTFGQGTKVEIK

SEQ ID NO: 62 - protein, 6 aa, synthetic construct
QSVSSN

SEQ ID NO: 63 - skipped

SEQ ID NO: 64 - protein, 9 aa, synthetic construct
QQYNNWPWT

SEQ ID NO: 65 - protein, 95 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWP

SEQ ID NO: 66 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 67 - protein, 7 aa, synthetic construct
QSVSSSY

SEQ ID NO: 68 - protein, 9 aa, synthetic construct
QQYGSSPWT

SEQ ID NO: 69 - protein, 120 aa, synthetic construct
EVQLVETGGGLIQPGGSLRLSCAASGFTVSSNYMSWVRQAPGKGLEWVSVIYSGGSTYYADSVKGRFTISRDNSKNTLYLQMNSLRAEDTAVYYCARGPTDAYPYLDYLWGQGTLVTVSS

SEQ ID NO: 70 - protein, 5 aa, synthetic construct
SNYMS

SEQ ID NO: 71 - protein, 16 aa, synthetic construct
VIYSGGSTYYADSVKG

SEQ ID NO: 72 - protein, 12 aa, synthetic construct
GPTDAYPYLDYL

SEQ ID NO: 73 - protein, 761 aa, Mus musculus
MKTWLKTVFGVTTLAALALVVICIVLRPSRVYKPEGNTKRALTLKDILNGTFSYKTYFPNWISEQEYLHQSEDDNIVFYNIETRESYIILSNSTMKSVNATDYGLSPDRQFVYLESDYSKLWRYSYTATYYIYDLQNGEFVRGYELPRPIQYLCWSPVGSKLAYVYQNNIYLKQRPGDPPFQITYTGRENRIFNGIPDWVYEEEMLATKYALWWSPDGKFLAYVEFNDSDIPIIAYSYYGDGQYPRTINIPYPKAGAKNPVVRVFIVDTTYPHHVGPMEVPVPEMIASSDYYFSWLTWVSSERVCLQWLKRVQNVSVLSICDFREDWHAWECPKNQEHVEESRTGWAGGFFVSTPAFSQDATSYYKIFSDKDGYKHIHYIKDTVENAIQITSGKWEAIYIFRVTQDSLFYSSNEFEGYPGRRNIYRISIGNSPPSKKCVTCHLRKERCQYYTASFSYKAKYYALVCYGPGLPISTLHDGRTDQEIQVLEENKELENSLRNIQLPKVEIKKLKDGGLTFWYKMILPPQFDRSKKYPLLIQVYGGPCSQSVKSVFAVNWITYLASKEGIVIALVDGRGTAFQGDKFLHAVYRKLGVYEVEDQLTAVRKFIEMGFIDEERIAIWGWSYGGYVSSLALASGTGLFKCGIAVAPVSSWEYYASIYSERFMGLPTKDDNLEHYKNSTVMARAEYFRNVDYLLIHGTADDNVHFQNSAQIAKALVNAQVDFQAMWYSDQNHGISSGRSQNHLYTHMTHFLKQCFSLSD

SEQ ID NO: 74 - protein, 459 aa, synthetic construct
QVQLQESGPGLVKPSETLSLTCTVSGGSISSNNYYWGWIRQTPGKGLEWIGSIYYSGSTNYNPSLKSRVTISVDTSKNQFSLKLSSVTAADTAVYYCARGARWQARPATRIDGVAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 213 aa, synthetic construct
EIVLTQSPGTLSLSPGERATLSCRASQTVTRNYLAWYQQKPGQAPRLLMYGASNRAAGVPDRFSGSGSGTDFTLTISRLEPEDFAVYYCQQFGSPYTFGQGTVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 76 - protein, 451 aa, synthetic construct
EVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDWSRSGYYLPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 219 aa, synthetic construct
DVVMTQSPLSLPVTLGQPASISCRSSQSLLHSNGYNYLDWYLQRPGQSPHLLIFLGSNRASGVPDRFSGSGSGTDFTLKISRVEAEDVGIYYCMQALQTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - protein, 456 aa, synthetic construct
EVQLVETGAEVKKPGASVKVSCKASDYIFTKYDINWVRQAPGQGLEWMGWMSANTGNTGYAQKFQGRVTMTRDTSINTAYMELSSLTSGDTAVYFCARSSLFKTETAPYYHFALDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 79 - protein, 125 aa, synthetic construct
EVQLVESGGGVVQPGRSLRLSCAASGFTFSNYGMHWVRQAPGKGLEWVAVISYDGSTKYSADSLKGRFTISRDNSKNTLYLQMNSLRADDTAVYYCAKEGWSFDSSGYRSWFDSWGQGTLVTVSS

(19) United States
(12) Patent Application Publication
(10) Pub. No.: US 2024/0218070 A1
(43) Pub. Date: Jul. 4, 2024

(54) FAP BINDING DOMAINS AND BISPECIFIC BINDING MOIETIES THAT BIND FAP AND TGF-ßRII

(71) Applicants: MERUS N.V., Utrecht (NL); INCYTE CORPORATION, Wilmington, DE (US)

(72) Inventors: Cecilia Anna Wilhelmina GEUIJEN, Utrecht (NL); Rinse KLOOSTER, Utrecht (NL); Amaya GARCIA DE VINUESA ANTUNANO, Utrecht (NL); Patrick MAYES, Wilmington, DE (US); Horatio G. NASTRI, Wilmington, DE (US); Stephen RUDNICK, Wilmington, DE (US); Bindu VARGHESE, Wilmington, DE (US); Moses DONKOR, Wilmington, DE (US)

(73) Assignees: MERUS N.V., Utrecht (NL); INCYTE CORPORATION, Wilmington, DE (US)

(21) Appl. No.: 18/545,263

(22) Filed: Dec. 19, 2023

(30) Foreign Application Priority Data
Dec. 20, 2022 (NL) 2033768
Dec. 20, 2022 (NL) 2033769

(51) Int. Cl. C07K 16/28; A61K 39/00; A61P 35/00; C07K 16/40

(52) U.S. Cl. CPC C07K 16/2863; A61P 35/00; C07K 16/2818; C07K 16/40; A61K 2039/505; C07K 2317/31; C07K 2317/524; C07K 2317/565; C07K 2317/732; C07K 2317/76; C07K 2317/92

(57) ABSTRACT

The present disclosure relates to polypeptides, FAP binding domains comprising such polypeptides, and binding domains comprising such FAP binding domains. The present disclosure further relates to the use of such binding domains or binding moieties in the treatment of cancer. This disclosure further relates to a bispecific binding moiety comprising a FAP binding domain and a TGF-βRII binding domain. This disclosure further relates to a pharmaceutical composition comprising an effective amount of said bispecific binding moiety, and to methods for treating a disease in a subject, comprising administering a therapeutically effective amount of said bispecific binding moiety.

### TECHNICAL FIELD

The present disclosure relates to the field of antibodies. In particular it relates to the field of therapeutic antibodies for the treatment of diseases involving aberrant cells. Particularly, it relates to binding domains that bind to FAP, and binding moieties comprising such FAP binding domains. It further relates to bispecific binding moieties comprising a binding domain that binds to FAP and a binding domain that binds to TGF-βRII.

### BACKGROUND

Cancer-associated fibroblasts (CAFs) have been identified as key players in promoting an immunosuppressive tumor microenvironment (TME) that blocks tumor infiltration of lymphocytes, and hence reduces the effectiveness of checkpoint blockade (Calon, A., et al. Dependency of Colorectal Cancer on a TGF-β-Driven Program in Stromal Cells for Metastasis Initiation. Cancer Cell. 2012 Nov. 13; 22(5):571-584). CAFs favor malignant progression by providing cancer cells with proliferative, migratory, survival and invasive capacities. Upstream TGF-β has been identified as critical in the activation of CAFs and maintenance of the TME.

TGF-β signaling regulates a plethora of normal physiological and pathological processes including cell cycle arrest in epithelial and hematopoietic cells, control of mesenchymal cell proliferation and differentiation, wound healing, extracellular matrix production, immunosuppression and carcinogenesis (Massagué J. TGFβ signalling in context. Nat Rev Mol Cell Biol. 2012 October; 13(10):616-30). In addition, TGF-β signaling regulates numerous cancer cell functions, including cell cycle progression, apoptosis, adhesion and differentiation (Liu S et al, Signal Transduction and targeted Therapy, 2021). TGF-β exhibits a biphasic function such that in normal and premalignant cells, it predominantly has been reported to act as a tumor suppressor, whereas in tumor cells it permits growth promoting functions, angiogenesis and epithelial-to-mesenchymal transition, which in turn permits tumor cell migration, invasion, intravasation and extravasation.

There are three TGF-β ligands (TGF-β1, 2 and 3) which mediate signaling through binding to TGF-β receptor type-2 (TGF-βRII), which leads to the dimerization with and phosphorylation of TGF-βRI. This heterotetrameric complex composed of two TGF-βRII and two TGF-βRI then recruits and phosphorylates SMAD2 and SMAD3, which in turn recruit and bind to the co-SMAD molecule SMAD4 to form the SMAD/co-SMAD complex, and translocates to the nucleus where it regulates the transcription of TGF-β target genes (Hata A, Chen Y G. TGF-β Signaling from Receptors to Smads. Cold Spring Harb Perspect Biol. 2016 Sep. 1; 8(9):a022061; Vander Ark A et al. TGF-β receptors: In and beyond TGF-β signaling. Cell Signal. 2018 December; 52:112-120). TGF-βRII is a member of the serine/threonine protein kinase family and the TGF-β receptor subfamily. It is known under various synonyms, including TGFBR2, AAT3, FAA3, LDS1B, LDS2, LDS2B, MFS2, RIIC, TAAD2, TGFR-2, TGFbeta-RII, transforming growth factor beta receptor 2, TBR-ii, and TBRII.

Certain moieties targeting the TGF-β pathway have been reported to show antitumor activity in vitro and in vivo; however, poor clinical outcomes persist with low efficacy and unacceptable toxicity, including major cytokine release syndrome (CRS). Combined targeting of the TGF-β pathway and immune checkpoint inhibition has been attempted with Bintrafusp alpha, an anti-PD-L1-TGF-βRII bifunctional fusion protein, without demonstrating a strong clinical effect and limiting toxicity.

Fibroblast activation protein (FAP) is a cell surface serine protease involved in the degradation of the extracellular matrix. It is known under various synonyms, including Seprase, DPPIV, FAPalpha, SIMP, Dipeptidyl Peptidase FAP, FAPA. FAP is not expressed by normal adult tissues; however, its expression is induced in activated fibroblasts during wound healing, stroma cells of epithelial cancers and some sarcomas (Kelly T. Fibroblast activation protein-alpha and dipeptidyl peptidase IV (CD26): cell-surface proteases that activate cell signaling and are potential targets for cancer therapy. Drug Resist Updat. 2005 February-April; 8(1-2):51-8). FAP is highly overexpressed on CAFs in the stroma of about 90% of all human epithelial cancers such as breast, lung and colorectal cancers. FAP is specifically upregulated by TGF-β. FAP degrades gelatin and type I collagen of the extracellular matrix due to its dipeptidyl peptidase and collagenolytic activity (Huber M A, et al. Fibroblast activation protein: differential expression and serine protease activity in reactive stromal fibroblasts of melanocytic skin tumors. J Invest Dermatol. 2003 February; 120(2):182-8). By degrading locally extracellular matrix components, FAP plays a critical role in cell migration and matrix invasion that occurs during tumor invasion, angiogenesis and metastasis.

Several anti-FAP antibodies have been studied in clinical trials. scFv M036 has been selected from FAP−/− immunized mice and is cross reactive to human FAP. M036 has been used to generate anti-FAP-CAR human T cells in an immunodeficient mouse model of human lung cancer. The anti-FAP antibody sibrotuzumab, which is the humanized version of the mouse monoclonal antibody F19, has been tested in a phase II clinical trial for metastatic CRC and a phase II clinical trial for NSCLC. Both trials have failed due to lack of therapeutic efficacy. A maytansinoid conjugate of the monoclonal antibody FAP5, FAP5-DM1, has been reported to inhibit tumor growth in xenograft models of lung, pancreas, and head and neck cancers.

There remains a need for novel therapeutic interventions that selectively target cancer-associated fibroblasts in the tumor microenvironment. Additionally, there remains a need for novel therapeutic interventions that selectively inhibit TGF-βRII signaling in the tumor microenvironment. Such targeting aims at inhibiting metastasis formation and restoration of tumor immunity, while limiting TGF-pathway blockade in normal tissues.

### SUMMARY

One of the objects of the present disclosure is to provide a new pharmaceutical agent for the treatment of human disease, in particular for the treatment of cancer. This object is met by the provision of FAP binding domains, and bispecific binding moieties comprising such FAP binding domains, for example bispecific antibodies, that bind FAP and TGF-βRII. The FAP binding domain of the bispecific binding moieties drives the specificity of the bispecific binding moiety to cancer associated fibroblasts (CAFs) in the tumor microenvironment (TME), where the TGF-βRII binding domain can locally block TGF-β from binding to TGF-βRII in the TME. Further, the bispecific binding moieties aim to promote cytotoxic T lymphocyte activity in the tumor microenvironment by alleviation of TGF-β-mediated immunosuppressive pathways on activated/exhausted effector T cells.

In certain embodiments, the present disclosure provides FAP binding domains comprising a polypeptide as further described herein that are particularly useful for the generation of binding moieties, such as antibodies.

In certain embodiments, the present disclosure provides a polypeptide selected from:
a polypeptide comprising a heavy chain CDR1 (HCDR1) having an amino acid sequence as set forth in SEQ ID NO: 16, a heavy chain CDR2 (HCDR2) having an amino acid sequence as set forth in SEQ ID NO: 17, and a heavy chain CDR3 (HCDR3) having an amino acid sequence as set forth in SEQ ID NO: 18;
a polypeptide comprising a heavy chain CDR1 (HCDR1) having an amino acid sequence as set forth in SEQ ID NO: 20, a heavy chain CDR2 (HCDR2) having an amino acid sequence as set forth in SEQ ID NO: 21, and a heavy chain CDR3 (HCDR3) having an amino acid sequence as set forth in SEQ ID NO: 22;
a polypeptide comprising a heavy chain CDR1 (HCDR1) having an amino acid sequence as set forth in SEQ ID NO: 12, a heavy chain CDR2 (HCDR2) having an amino acid sequence as set forth in SEQ ID NO: 13, and a heavy chain CDR3 (HCDR3) having an amino acid sequence as set forth in SEQ ID NO: 14; or
a polypeptide comprising a heavy chain CDR1 (HCDR1) having an amino acid sequence as set forth in SEQ ID NO: 70, a heavy chain CDR2 (HCDR2) having an amino acid sequence as set forth in SEQ ID NO: 71, and a heavy chain CDR3 (HCDR3) having an amino acid sequence as set forth in SEQ ID NO: 72.

In certain embodiments, the present disclosure provides a FAP binding domain comprising a polypeptide as described herein.

In certain embodiments, the present disclosure provides a FAP binding domain that binds to human FAP and mouse FAP.

In certain embodiments, the present disclosure provides a binding moiety comprising a polypeptide, or a FAP binding domain, as described herein.

In certain embodiments, the present disclosure provides a pharmaceutical composition comprising an effective amount of a polypeptide, or of a FAP binding domain, or of a binding moiety, as described herein, and a pharmaceutically acceptable carrier.

In certain embodiments, the present disclosure provides a polypeptide, or a FAP binding domain, or a binding moiety, or a pharmaceutical composition, as described herein, for use in therapy.

In certain embodiments, the present disclosure provides a polypeptide, or a FAP binding domain, or a binding moiety, or a pharmaceutical composition, as described herein, for use in the treatment of cancer.

In certain embodiments, the present disclosure provides a method for treating a disease, comprising administering an effective amount of a polypeptide, or of a FAP binding domain, or of a binding moiety, or of a pharmaceutical composition, as described herein, to an individual in need thereof.

In certain embodiments, the present disclosure provides a method for treating cancer, comprising administering an effective amount of a polypeptide, or of a FAP binding domain, or of a binding moiety, or of a pharmaceutical composition, as described herein, to an individual in need thereof.

In certain embodiments, the present disclosure provides a nucleic acid comprising a sequence that encodes a polypeptide as described herein.

In certain embodiments, the present disclosure provides a vector comprising a nucleic acid sequence as described herein.

In certain embodiments, the present disclosure provides a cell comprising a nucleic acid as described herein.

In certain embodiments, the present disclosure provides a cell producing a polypeptide, or a FAP binding domain, or a binding moiety, as described herein.

In certain embodiments, the present disclosure provides a bispecific binding moiety comprising a FAP binding domain and a TGF-βRII binding domain, wherein the TGF-βRII binding domain blocks TGF-βRII-mediated signaling.

In certain embodiments, the present disclosure provides a bispecific binding moiety comprising a FAP binding domain and a TGF-βRII binding domain, wherein the FAP binding domain binds to FAP expressed on a first cell and the TGF-βRII binding domain binds to TGF-βRII expressed on a second cell.

In certain embodiments, the present disclosure provides a bispecific binding moiety comprising a FAP binding domain and a TGF-βRII binding domain, wherein the FAP binding domain comprises a heavy chain variable region comprising CDR1, CDR2, and CDR3 sequences as described further herein.

In certain embodiments, the present disclosure provides a bispecific binding moiety comprising a FAP binding domain and a TGF-βRII binding domain, wherein the TGF-βRII binding domain comprises a heavy chain variable region comprising CDR1, CDR2, and CDR3 sequences as described further herein.

In certain embodiments, the present disclosure provides a pharmaceutical composition comprising an effective amount of a bispecific binding moiety as described herein.

In certain embodiments, the present disclosure provides a bispecific binding moiety as described herein, and a pharmaceutical composition as described herein, for use in therapy.

In certain embodiments, the present disclosure provides a bispecific binding moiety as described herein, and a pharmaceutical composition as described herein, for use in the treatment of cancer.

In certain embodiments, the present disclosure provides a combination of a bispecific binding moiety as described herein and a second binding moiety that binds PD-1 for use in therapy.

In certain embodiments, the present disclosure provides a combination of a bispecific binding moiety as described herein and a second binding moiety that binds PD-1 for use in the treatment of cancer.

In certain embodiments, the present disclosure provides a method for treating a disease, comprising administering an effective amount of a bispecific binding moiety as described herein, or a pharmaceutical composition as described herein, to an individual in need thereof.

In certain embodiments, the present disclosure further provides a method for treating cancer, comprising administering an effective amount of a bispecific binding moiety as described herein, or a pharmaceutical composition as described herein, to an individual in need thereof.

In certain embodiments, the present disclosure further provides a nucleic acid sequence encoding a heavy chain variable region of a FAP binding domain as described herein.

In certain embodiments, the present disclosure further provides a nucleic acid sequence encoding a heavy chain variable region of a FAP binding domain and a heavy chain variable region of a TGF-βRII binding domain as described herein.

In certain embodiments, the present disclosure provides a cell comprising a nucleic acid sequence encoding the heavy chain variable region of a FAP binding domain as described herein and a nucleic acid sequence encoding the heavy chain variable region of a TGF-βRII binding domain as described herein.

In certain embodiments, the present disclosure further provides a cell producing a bispecific binding moiety as described herein.

In certain embodiments, the present disclosure further provides a bispecific binding moiety that competes with a bispecific binding moiety as described herein for binding to FAP and TGF-βRII.

### DETAILED DESCRIPTION

### FAP Binding Domains

One of the objects of the present disclosure is to provide a binding domain that binds to human FAP for use in the development of a new pharmaceutical agent for the diagnosis and treatment of disease, in particular in humans, and in particular for the diagnosis and treatment of cancer. This object is met by the provision of binding domains that comprise a polypeptide identified herein as being useful for the generation of antibodies, and in particular for the generation of bispecific antibodies.

In certain embodiments, the present disclosure provides a polypeptide selected from:
a polypeptide comprising a heavy chain CDR1 (HCDR1) having an amino acid sequence as set forth in SEQ ID NO: 16, a heavy chain CDR2 (HCDR2) having an amino acid sequence as set forth in SEQ ID NO: 17, and a heavy chain CDR3 (HCDR3) having an amino acid sequence as set forth in SEQ ID NO: 18;
a polypeptide comprising a heavy chain CDR1 (HCDR1) having an amino acid sequence as set forth in SEQ ID NO: 20, a heavy chain CDR2 (HCDR2) having an amino acid sequence as set forth in SEQ ID NO: 21, and a heavy chain CDR3 (HCDR3) having an amino acid sequence as set forth in SEQ ID NO: 22;
a polypeptide comprising a heavy chain CDR1 (HCDR1) having an amino acid sequence as set forth in SEQ ID NO: 12, a heavy chain CDR2 (HCDR2) having an amino acid sequence as set forth in SEQ ID NO: 13, and a heavy chain CDR3 (HCDR3) having an amino acid sequence as set forth in SEQ ID NO: 14; or
a polypeptide comprising a heavy chain CDR1 (HCDR1) having an amino acid sequence as set forth in SEQ ID NO: 70, a heavy chain CDR2 (HCDR2) having an amino acid sequence as set forth in SEQ ID NO: 71, and a heavy chain CDR3 (HCDR3) having an amino acid sequence as set forth in SEQ ID NO: 72.

In certain embodiments, the polypeptide is an immunoglobulin heavy chain, or part thereof, that, when combined with a suitable light chain, or part thereof, binds to FAP. For example, the part of an immunoglobulin heavy chain can be a heavy chain variable region with a CH1 region, or a heavy chain variable region. The part of a light chain can, for example, be a light chain variable region.

In certain embodiments, the polypeptide when combined with a suitable light chain, or part thereof, binds to human FAP. The amino acid sequence of human FAP is provided as SEQ ID NO: 6. The intracellular and transmembrane domains are indicated in bold and underlined therein. In certain embodiments, the polypeptide when combined with a suitable light chain, or part thereof, binds to mouse FAP. The amino acid sequence of mouse FAP is provided as SEQ ID NO: 73. The intracellular and transmembrane domains are indicated in bold and underlined therein. In certain embodiments, the polypeptide when combined with a suitable light chain, or part thereof, binds to cynomolgus FAP. The amino acid sequence of cynomolgus FAP is provided as SEQ ID NO: 9. The intracellular and transmembrane domains are indicated in bold and underlined therein. In certain embodiments, the polypeptide when combined with a suitable light chain, or part thereof, binds to human and mouse FAP. In certain embodiments, the polypeptide when combined with a suitable light chain, or part thereof, binds to human and cynomolgus FAP. In certain embodiments, the polypeptide when combined with a suitable light chain, or part thereof, binds to human, mouse, and cynomolgus FAP.

In general, as described herein, antigen binding can be expressed in terms of specificity and affinity. The specificity determines which antigen or epitope thereof is specifically bound by a binding domain or binding moiety. The affinity is a measure for the strength of binding to a particular antigen or epitope.

In certain embodiments, a polypeptide of the present disclosure also includes polypeptide variants thereof, wherein each of the HCDR1 or HCDR2 may comprise at most three, two, or one amino acid variations. In certain embodiments, only one of the HCDR1 or HCDR2 may comprise at most three, two, or one amino acid variations. In certain embodiments, such variants do not comprise amino acid variations in the HCDR3. In certain embodiments, the amino acid variation is a conservative amino acid substitution.

In general, as described herein, typically, a conservative amino acid substitution involves a variation of an amino acid with a homologous amino acid residue, which is a residue that shares similar characteristics or properties. Homologous amino acids are known in the art, as are routine methods for making amino acid substitutions in antibody binding domains without significantly impacting binding or function of the antibody, see for instance handbooks like Lehninger (Nelson, David L., and Michael M. Cox. 2017. Lehninger Principles of Biochemistry. 7th ed. New York, NY: W.H. Freeman) or Stryer (Berg, J., Tymoczko, J., Stryer, L. and Stryer, L., 2007. Biochemistry. New York: W.H. Freeman), incorporated herein in its entirety. In determining whether an amino acid can be replaced with a conserved amino acid, an assessment may typically be made of factors such as, but not limited to, (a) the structure of the polypeptide backbone in the area of the substitution, for example, a sheet or helical conformation, (b) the charge or hydrophobicity of the molecule at the target site, and/or (c) the bulk of the side chain(s). If a residue can be substituted with a residue which has common characteristics, such as a similar side chain or similar charge or hydrophobicity, then such a residue is preferred as a substitute. For example, the following groups can be determined: (1) non-polar: Ala (A), Gly (G), Val (V), Leu (L), Ile (I), Pro (P), Phe (F), Trp (W), Met (M); (2) uncharged polar: Ser (S), Thr (T), Cys (C), Tyr (Y), Asn (N), Gln (Q); (3) acidic: Asp (D), Glu (E); and (4) basic: Lys (K), Arg (R), His (H). Alternatively, the amino acids may be grouped as follows: (1) aromatic: Phe (F), Trp (W), Tyr (Y); (2) apolar: Leu (L), Val (V), Ile (I), Ala (A), Met (M); (3) aliphatic: Ala (A), Val (V), Leu (L), Ile (I); (4) acidic: Asp (D), Glu (E); (5) basic: His (H), Lys (K), Arg (R); and (6) polar: Gln (Q), Asn (N), Ser (S), Thr (T), Tyr (Y). Alternatively, amino acid residues may be divided into groups based on common side-chain properties: (1) hydrophobic: Met (M), Ala (A), Val (V), Leu (L), Ile (I); (2) neutral hydrophilic: Cys (C), Ser (S), Thr (T), Asn (N), Gln (Q); (3) acidic: Asp (D), Glu (E); (4) basic: His (H), Lys (K), Arg R); (5) residues that influence chain orientation: Gly (G), Pro (P); and (6) aromatic: Trp (W), Tyr (Y), Phe (F).

The substitution of an amino acid residue with another present in the same group would be preferred. Accordingly, conservative amino acid substitution can involve exchanging a member of one of these classes for another member of that same class. Typically, the variation results in no, or substantially no, loss in binding specificity of the binding domain to its intended target.

Additional types of amino acid variations include variations resulting from somatic hypermutation or affinity maturation. Binding variants encompassed by the present disclosure include somatically hypermutated or affinity matured heavy chain variable regions, which are heavy chain variable regions derived from the same VH gene segments as the heavy chain variable regions described by sequence herein, the variants having amino acid variations, including non-conservative and/or conservative amino acid substitutions in one or two of HCDR1 and HCDR2. Routine methods for affinity maturing antibody binding domains are widely known in the art, see for instance Tabasinezhad M, et al. (Trends in therapeutic antibody affinity maturation: From in-vitro towards next-generation sequencing approaches. Immunol Lett. 2019 August; 212:106-113).

In certain embodiments, a polypeptide of the present disclosure comprises a heavy chain variable region having an amino acid sequence as set forth in SEQ ID NO: 11; 15; 19; or 69, or having at least 80%, or at least 85%, or at least 90%, or at least 95%, sequence identity thereto.

In general, as described herein, “percent (%) identity” as referring to nucleic acid or amino acid sequences herein is defined as the percentage of residues in a candidate sequence that are identical with the residues in a selected sequence, after aligning the sequences for optimal comparison purposes. In order to optimize the alignment between the two sequences gaps may be introduced in any of the two sequences that are compared. Such alignment can be carried out over the full length of the sequences being compared. Alternatively, the alignment may be carried out over a shorter length, for example over about 20, about 50, about 100 or more nucleic acids/bases or amino acids. The sequence identity is the percentage of identical matches between the two sequences over the reported aligned region.

A comparison of sequences and determination of percentage of sequence identity between two sequences can be accomplished using a mathematical algorithm. The skilled person will be aware of the fact that several different computer programs are available to align two sequences and determine the identity between two sequences (Kruskal, J. B. (1983) An overview of sequence comparison In D. Sankoff and J. B. Kruskal, (ed.), Time warps, string edits and macromolecules: the theory and practice of sequence comparison, pp. 1-44 Addison Wesley). The percent sequence identity between two amino acid sequences or nucleic acid sequences may be determined using the Needleman and Wunsch algorithm for the alignment of two sequences. (Needleman, S. B. and Wunsch, C. D. (1970) J. Mol. Biol. 48, 443-453). The Needleman-Wunsch algorithm has been implemented in the computer program NEEDLE. For the purpose of this disclosure, the NEEDLE program from the EMBOSS package is used to determine percent identity of amino acid and nucleic acid sequences (version 2.8.0, EMBOSS: The European Molecular Biology Open Software Suite (2000) Rice, P. Longden J. and Bleasby, A. Trends in Genetics 16, (6) pp 276-277, http://emboss.bioinformatics.nl/). For protein sequences, EBLOSUM62 is used for the substitution matrix. For DNA sequences, DNAFULL is used. The parameters used are a gap-open penalty of 10 and a gap extension penalty of 0.5.

After alignment by the program NEEDLE as described above the percentage of sequence identity between a query sequence and a sequence of the invention is calculated as follows: Number of corresponding positions in the alignment showing an identical amino acid or identical nucleotide in both sequences divided by the total length of the alignment after subtraction of the total number of gaps in the alignment.

In certain embodiments, a polypeptide of the present disclosure also comprises polypeptide variants, which, in addition to variations in the HCDR1 and/or HCDR2 referred to above, comprise one or more variations in the framework regions. A variation can be any type of amino acid variation described herein, such as for instance a conservative amino acid substitution or non-conservative amino acid substitution resulting from somatic hypermutation or affinity maturation. In certain embodiments, a polypeptide of the present disclosure comprises no variations in the CDR regions but comprises one or more variations in the framework regions. Such variants have at least 80%, at least 85%, at least 90%, or at least 95% sequence identity to the sequences disclosed herein. Such variants are expected to retain FAP binding specificity. Thus, in certain embodiments, a polypeptide of the present disclosure comprises:
a polypeptide having at least 80%, at least 85%, at least 90%, or at least 95% sequence identity to the amino acid sequence as set forth in SEQ ID NO: 15, which polypeptide comprises a HCDR1 amino acid sequence as set forth in SEQ ID NO: 16; a HCDR2 amino acid sequence as set forth in SEQ ID NO: 17; and a HCDR3 amino acid sequence as set forth in SEQ ID NO: 18;
a polypeptide having at least 80%, at least 85%, at least 90%, or at least 95% sequence identity to the amino acid sequence as set forth in SEQ ID NO: 19, which polypeptide comprises a HCDR1 amino acid sequence as set forth in SEQ ID NO: 20; a HCDR2 amino acid sequence as set forth in SEQ ID NO: 21; and a HCDR3 amino acid sequence as set forth in SEQ ID NO: 22;
a polypeptide having at least 80%, at least 85%, at least 90%, or at least 95% sequence identity to the amino acid sequence as set forth in SEQ ID NO: 11, which polypeptide comprises a HCDR1 amino acid sequence as set forth in SEQ ID NO: 12; a HCDR2 amino acid sequence as set forth in SEQ ID NO: 13; and a HCDR3 amino acid sequence as set forth in SEQ ID NO: 14; or
a polypeptide having at least 80%, at least 85%, at least 90%, or at least 95% sequence identity to the amino acid sequence as set forth in SEQ ID NO: 69, which polypeptide comprises a HCDR1 amino acid sequence as set forth in SEQ ID NO: 70; a HCDR2 amino acid sequence as set forth in SEQ ID NO: 71; and a HCDR3 amino acid sequence as set forth in SEQ ID NO: 72.

In certain embodiments, the polypeptides of the present disclosure have been generated with the light chain VK1-39/JK1. A polypeptide of the present disclosure may be paired with any suitable light chain. In certain embodiments, a suitable light chain is light chain VK1-39/JK1. This light chain comprises a light chain variable region comprising light chain CDR1 (LCDR1), light chain CDR2 (LCDR2), and light chain CDR3 (LCDR3), having an amino acid sequence as set forth in SEQ ID NO: 53, SEQ ID NO: 54, and SEQ ID NO: 55. In certain embodiments, a suitable light chain comprises a light chain variable region having an amino acid sequence as set forth in SEQ ID NO: 52.

In certain embodiments, a polypeptide of the present disclosure further comprises a CH1 region. In certain embodiments, a polypeptide of the present disclosure further comprises a CH1 region, hinge, CH2 region, and CH3 region. A suitable CH1 region includes, but is not limited to, the CH1 region of which the amino acid sequence is set forth in SEQ ID NO: 39. A suitable hinge includes, but is not limited to, the hinge of which the amino acid sequence is set forth in SEQ ID NO: 40. Suitable CH2 and CH3 regions include, but are not limited to, the CH2 region of which the amino acid sequence is set forth in SEQ ID NO: 41 (WT) or 42 (DM), and the CH3 region of which the amino acid sequence is set forth in SEQ ID NO: 43 (WT), or 44 (DE) and 45 (KK).

In certain embodiments, the present disclosure provides a FAP binding domain comprising a polypeptide as described herein.

In certain embodiments, the present disclosure provides a FAP binding domain that binds to human FAP and mouse FAP, i.e. a FAP binding domain that is cross-reactive for human and mouse FAP. In certain embodiments, the human/mouse cross-reactive FAP binding domain has a binding affinity for human FAP that is at least 2-3 times higher than the background signal of the assay. In certain embodiments, the human/mouse cross-reactive FAP binding domain has a binding affinity for mouse FAP that is at least 2-3 times higher than the background signal of the assay. In certain embodiments, the human/mouse cross-reactive FAP binding domain has a binding affinity for human FAP that is at least 2 times higher than the background signal of the assay. In certain embodiments, the human/mouse cross-reactive FAP binding domain has a binding affinity for mouse FAP that is at least 2 times higher than the background signal of the assay.

For the purpose of the present disclosure, in certain embodiments, binding to huFAP or moFAP is determined by using the assays as described in Example 2. In certain embodiments, binding to huFAP is determined using a FACS assay with cells expressing human FAP. In certain embodiments, binding to moFAP is determined using a FACS assay with cells expressing mouse FAP.

In certain embodiments, a human/mouse cross-reactive FAP binding domain comprises a polypeptide comprising a heavy chain CDR1 (HCDR1) having an amino acid sequence as set forth in SEQ ID NO: 70, a heavy chain CDR2 (HCDR2) having an amino acid sequence as set forth in SEQ ID NO: 71, and a heavy chain CDR3 (HCDR3) having an amino acid sequence as set forth in SEQ ID NO: 72.

In certain embodiments, a human/mouse cross-reactive FAP binding domain of the present disclosure comprises a heavy chain variable region having an amino acid sequence as set forth in SEQ ID NO: 69, or having at least 80%, or at least 85%, or at least 90%, or at least 95%, sequence identity thereto.

In certain embodiments, a human/mouse FAP binding domain of the present disclosure also comprises polypeptide variants. Polypeptide variants include polypeptides comprising variations in the HCDR1, HCDR2, and/or framework regions as described herein.

In certain embodiments, a human/mouse FAP binding domain of the present disclosure further comprises a light chain as defined herein.

In certain embodiments, a human/mouse FAP binding domain of the present disclosure further comprises a CH1 region as defined herein. In certain embodiments, a human/mouse FAP binding domain of the present disclosure further comprises a CH1 region, hinge, CH2 region, and CH3 region, as defined herein.

A CL, CH1, hinge, CH2, and/or CH3 region may be modified according to methods known in the art in order to obtain favorable antibody characteristics, including for instance to promote heterodimerization of different heavy chains, to improve heavy-light chain pairing, and to enhance or reduce immune cell effector function. A CH3 region may comprise the terminal lysine residue, or lack the terminal lysine residue to improve manufacturability.

In certain embodiments, the present disclosure provides a binding moiety comprising a polypeptide or a FAP binding domain as described herein. This binding moiety is also referred to herein as a FAP binding moiety.

In general, as described herein, a “binding moiety” refers to a proteinaceous molecule and includes for instance all antibody formats available in the art, such as for example a full length IgG antibody, immunoconjugates, diabodies, BiTEs, Fab fragments, scFv, tandem scFv, single domain antibody (like VHH and VH), minibodies, scFab, scFv-zipper, nanobodies, DART molecules, TandAb, Fab-scFv, F(ab)′2, F(ab)′2-scFv2, and intrabodies, as well as any other formats known to a person of ordinary skill in the art.

In certain embodiments, a binding moiety of the present disclosure is a monospecific binding moiety, in particular a monospecific antibody. A monospecific antibody according to the present disclosure is an antibody, in any antibody format, that comprises one or more binding domains with specificity for a single target. In certain embodiments, a monospecific binding moiety of the present disclosure may further comprise an Fc region or a part thereof. In certain embodiments, a monospecific binding moiety of the present disclosure is an IgG1 antibody. In certain embodiments, a binding moiety of the present disclosure is a bivalent monospecific antibody.

In general, as described herein, an “Fc region” typically comprises a hinge, CH2, and CH3 region. Suitable hinge, CH2, and CH3 regions are as described herein. The Fc region mediates effector functions of an antibody, such as complement-dependent cytotoxicity (CDC), antibody-dependent cellular cytotoxicity (ADCC) and antibody-dependent cell phagocytosis (ADCP). Depending on the therapeutic antibody or Fc fusion protein application, it may be desired to either reduce or increase the effector function.

In certain embodiments, a binding moiety comprising a polypeptide or binding domain of the present disclosure has Fc effector function. In certain embodiments, a binding moiety comprising a polypeptide or binding domain of the present disclosure has enhanced Fc effector function. In certain embodiments, a binding moiety comprising a polypeptide or binding domain of the present disclosure exhibits antibody-dependent cell-mediated cytotoxicity (ADCC).

In general, as described herein, a binding moiety, such as an antibody, can be engineered to enhance the ADCC activity (for review, see Kubota T et al. Cancer Sci. 2009; 100(9):1566-72). For instance, ADCC activity of an antibody can be improved when the antibody itself has a low ADCC activity, by slightly modifying the constant region of the antibody (Junttila T T. et al. Cancer Res. 2010; 70(11):4481-9). Changes are sometimes also made to improve storage or production or to remove C-terminal lysins (Kubota T et al. Cancer Sci. 2009; 100(9):1566-72). Another way to improve ADCC activity of an antibody is by enzymatically interfering with the glycosylation pathway resulting in a reduced fucose (von Horsten H H. et al. Glycobiology. 2010; 20(12): 1607-18). Alternatively, or additionally, multiple other strategies can be used to achieve ADCC enhancement, for instance including glycoengineering (Kyowa Hakko/Biowa, GlycArt (Roche) and Eureka Therapeutics) and mutagenesis, all of which seek to improve Fc binding to low-affinity activating FcγRIIIa, and/or to reduce binding to the low affinity inhibitory FcγRIIb. In certain embodiments, a binding moiety of the present disclosure exhibits enhanced antibody-dependent cell-mediated cytotoxicity (ADCC).

In certain embodiments, a FAP binding moiety of the present disclosure is afucosylated.

In general, as described herein, afucosylation of antibodies can be obtained using various methods known in the art. Fc-enhanced variants of antibodies can be produced using FUT-8 knock-out CHO cells, which generates afucosylated antibodies (Zong H, et al. Producing defucosylated antibodies with enhanced in vitro antibody-dependent cellular cytotoxicity via FUT8 knockout CHO-S cells. Eng Life Sci. 2017 Apr. 18; 17(7):801-808). Afucosylation of antibodies can also be achieved using CHO cells expressing GDP-6-deoxy-D-lyxo-4-hexulose reductase (RMD) enzyme (Roy G, et. al., A novel bicistronic gene design couples stable cell line selection with a fucose switch in a designer CHO host to produce native and afucosylated glycoform antibodies, MAbs, 2018 April; 10(3):416-430).

Bispecific Binding Moieties that Bind FAP and TGF-βRII

Another of the objects of the present disclosure is to provide a new pharmaceutical agent for the diagnosis and treatment of disease, in particular in humans and in particular for the diagnosis and treatment of cancer. This object is met by the provision of bispecific binding moieties, for example bispecific antibodies, that bind FAP and TGF-βRII. The FAP binding domain of the bispecific binding moieties drives the specificity of the bispecific binding moiety to cancer associated fibroblasts (CAFs) in the tumor microenvironment, where the TGF-βRII binding domain can locally block TGF-β from binding to TGF-βRII. Further, the bispecific binding moieties promote cytotoxic T lymphocyte activity in the tumor microenvironment by alleviation of TGF-β-mediated immunosuppressive pathways in activated/exhausted effector T cells.

In certain embodiments, the present disclosure provides a bispecific binding moiety comprising a FAP binding domain and a TGF-βRII binding domain, wherein the TGF-βRII binding domain blocks TGF-βRII binding to TGF-βRII ligand.

In certain embodiments, the bispecific binding moiety of the present disclosure is a bispecific antibody. A bispecific antibody according to the present disclosure is an antibody that comprises at least two binding domains which have specificity for at least two different targets or epitopes. In certain embodiments, a bispecific antibody of the present disclosure is a bivalent bispecific antibody. In certain embodiments, a bispecific antibody of the present disclosure further comprises an Fc region or a part thereof. In certain embodiments, a bispecific binding moiety of the present disclosure is an IgG1 antibody. Constant regions of a binding moiety of the present disclosure may comprise one or more variations that modulate properties of the binding moiety other than its binding properties to the target antigens. For instance, the constant regions may comprise one or more variations that promote heterodimerization of the FAP and TGF-βRII heavy chains over homodimerization of two FAP heavy chains and/or two TGF-βRII heavy chains, one or more variations in the CH1 and/or CL that improve heavy-light chain pairing, and/or the constant regions may comprise one or more variations that reduce or improve effector function, in particular one or more variations that reduce effector function.

In certain embodiments, a FAP binding domain and/or TGF-βRII binding domain is a Fab domain, also referred to as “Fab” herein. For the purpose of the present disclosure, a “Fab” means a binding domain comprising a heavy chain variable region, a light chain variable region, a CH1 and a CL region.

In certain embodiments, a bispecific binding moiety of the present disclosure comprises a single Fab domain that binds to FAP, a single Fab domain that binds to TGF-βRII, and an Fc region. In certain embodiments, a bispecific binding moiety of the present disclosure consists of a single Fab domain that binds to FAP, a single Fab domain that binds to TGF-βRII, and an Fc region.

For the purpose of the present disclosure, an “Fc region” comprises a hinge, CH2, and CH3 region. A suitable hinge includes, but is not limited to, the hinge of which the amino acid sequence is set forth in SEQ ID NO: 40. Suitable CH2 and CH3 regions include, but are not limited to the CH2 region of which the amino acid sequence is set forth in SEQ ID NO: 41 or 42, and the CH3 region of which the amino acid sequence is set forth in SEQ ID NO: 43, or 44 and 45. A CH3 region may comprise the terminal lysine residue, or lack the terminal lysine residue to improve manufacturability.

In certain embodiments, the bispecific binding moiety of the present disclosure binds to human FAP. The amino acid sequence of human FAP is provided as SEQ ID NO: 6. In certain embodiments, the bispecific binding moiety of the present disclosure has a binding affinity for human FAP that is at least 2 times higher than the background signal of the assay.

For the purpose of the present disclosure, in certain embodiments, binding to huFAP is determined by using the assays as described in Example 2. In certain embodiments, binding to huFAP is determined using a FACS assay with cells expressing human FAP.

In certain embodiments, the FAP binding domain of the bispecific binding moiety disclosed herein, binds to human FAP and is cross-reactive with cynomolgus FAP (cyFAP). The amino acid sequence of cyFAP is provided as SEQ ID NO: 9. In certain embodiments, the bispecific binding moiety of the present disclosure has a binding affinity for cynomolgus FAP that is at least 2 times higher than the background signal of the assay.

For the purpose of the present disclosure, in certain embodiments, binding to cyFAP is determined by using the assays as described in Example 2. In certain embodiments, binding to cyFAP is determined using a FACS assay with cells expressing cynomolgus FAP.

In certain embodiments, the FAP binding domain of the bispecific binding moiety disclosed herein, does not bind to human CD26 (huCD26). The amino acid sequence of huCD26 is provided as SEQ ID NO: 10. In certain embodiments, the bispecific binding moiety of the present disclosure has a binding affinity for human CD26 that is equal to or lower than the background signal of the assay. In certain embodiments, binding to huCD26 is determined using a FACS assay with cells expressing huCD26.

In certain embodiments, the bispecific binding moiety of the present disclosure binds to human TGF-βRII. Human TGF-βRII is a transmembrane protein of which there are different isoforms. The amino acid sequence of human TGF-βRII isoform A is provided as SEQ ID NO: 46; the amino acid sequence of the extracellular domain of human TGF-βRII isoform A is provided as SEQ ID NO: 47. Human TGF-βRII isoform B is a splice variant encoding a longer isoform due to an insertion in the extracellular domain. The amino acid sequence of human TGF-βRII isoform B is provided as SEQ ID NO: 48; the amino acid sequence of the extracellular domain of isoform B of human TGF-βRII is as set forth in SEQ ID NO: 49. In certain embodiments, the bispecific binding moiety of the present disclosure binds to isoform A of human TGF-βRII. In certain embodiments, the bispecific binding moiety of the present disclosure has a binding affinity for human TGF-βRII that is at least 2 times higher than the background signal of the assay. In certain embodiments, binding to human TGF-βRII is determined with a FACS assay using cells expressing human TGF-βRII, such as for instance cells endogenously expressing human TGF-βRII, for example CCD18Co cells.

In certain embodiments, TGF-βRII ligand is TGF-β1.

In certain embodiments, the TGF-βRII binding domain of the bispecific binding moiety of the present disclosure blocks binding of TGF-βRII to TGF-βRII ligand TGF-βI.

As used herein, “blocks TGF-βRII binding to TGF-βRII ligand” or “blocking TGF-βRII binding to TGF-βRII ligand” means interfering or modifying the interaction between a ligand of TGF-βRII and a TGF-βRII receptor. This occurs when the TGF-βRII binding domain of the bispecific binding moiety is directed to an epitope on TGF-βRII and competes with TGF-β1 for binding to human TGF-βRII. In certain embodiments, blocking TGF-βRII binding to TGF-βRII ligand is determined by using an ELISA assay as described in the art, for example in WO 2021/133167.

In certain embodiments, the TGF-βRII binding domain of the bispecific binding moiety blocks TGF-βRII mediated signaling in a cell expressing FAP and TGF-βRII.

As used herein, “blocks TGF-βRII mediated signaling” or “blocking TGF-βRII mediated signaling” means causing a total or partial reduction of the signal transduction cascade. For the purpose of the present disclosure, in certain embodiments, blocking TGF-βRII mediated signaling is determined by using a TGF-βRII signaling inhibition assay as described in Example 7. The TGF-βRII-mediated signaling inhibition data of the bispecific binding moieties as provided herein is obtained using the assay as described in Example 7.

In brief, the TGF-βRII signaling inhibition assay in Example 7 is performed using primary CAF cells, which are trypsinized and re-suspended in a suitable buffer. Cells are pre-incubated with the test bispecific binding moieties, followed by incubation with recombinant human TGF-β1 and then subsequently assayed for pSMAD2 expression. The potency in blocking TGF-βRII mediated signaling is determined in IC50 (ug/ml).

In certain embodiments, reduction in pSMAD2 expression is determined in IC50 (ug/ml), wherein pSMAD2 expression in the presence of the bispecific binding moiety is compared to pSMAD2 expression in the absence of the bispecific binding moiety, in a TGF-βRII signaling inhibition assay.

In certain embodiments, a cell expressing both FAP and TGF-βRII is a fibroblast, in particular a primary cancer associated fibroblast (CAF), such as for instance, primary human lung squamous cell cancer CAF, primary human bladder CAF, primary human breast CAF, primary human head and neck CAF, primary colon cancer CAF, primary pancreatic stellate CAF, primary melanoma CAF, primary lung adenocarcinoma CAF, primary colorectal adenocarcinoma CAF, primary ovarian serous CAF, or primary glioblastoma CAF. Primary CAF cells expressing FAP and TGF-βRII are commercially available from for instance, BioIVT or Neuromics, as described in Table 3, and Example 6. Alternatively, methods for producing cells expressing FAP and TGF-βRII are known in the art to persons of ordinary skill. Methods for determining expression of FAP and TGF-βRII on cells are known to a skilled person. In certain embodiments, expression of FAP and TGF-βRII on CAF cells is determined as Mean Fluorescence Intensity (MFI) by using a FACS assay as described in Example 6. In certain embodiments, the MFI of FAP and TGF-βRII expression is at least 2 fold or 3 fold higher than the background MFI obtained with only the secondary antibody as set out in Example 6.

In Example 6, CAF cells are cultured and re-suspended in a suitable buffer and stained with primary antibody. For huFAP detection, mouse anti-FAP antibody is used as primary antibody and for huTGF-βRII detection, analog reference TGF1 antibody is used as primary antibody. After washing of the primary antibody, cells are stained with a suitable secondary antibody for instance, FITC-conjugated goat anti-mouse antibody and Alexa Fluor 647 conjugated goat anti-human antibody. Stained cells are analyzed in FACS and expression levels are determined as MFI.

In certain embodiments, the potency of a bispecific binding moieties of the present disclosure in blocking TGF-βRII mediated signaling is 2.0-500 fold higher than the potency of a reference anti-TGF-βRII antibody in a cell expressing FAP and TGF-βRII.

In certain embodiments, the potency in blocking TGF-βRII mediated signaling is determined by measuring reduction in pSMAD2 expression, in IC50 (ug/ml), as described in Example 7. In certain embodiments, the potency in blocking TGF-βRII mediated signaling is determined as reduction in pSMAD2 expression, in IC50 (ug/ml).

In certain embodiments, a bispecific binding moiety of the present disclosure has a potency in blocking TGF-βRII mediated signaling in a range of about 2.0-500, or in a range of 2.0-300, fold higher than the potency of a reference anti-TGF-βRII antibody in a cell expressing FAP and TGF-βRII, as measured as reduction in pSMAD2 expression as described in Example 7. In certain embodiments, a bispecific binding moiety of the present disclosure has a potency in blocking TGF-βRII mediated signaling in a range of about 2.0-5, or about 2, fold higher than the potency of a reference anti-TGF-βRII antibody in a cell expressing FAP and TGF-βRII, as measured as reduction in pSMAD2 expression as described in Example 7. In certain embodiments, a bispecific binding moiety of the present disclosure has a potency in blocking TGF-βRII mediated signaling in a range of about 50-100, or about 80, fold higher than the potency of a reference anti-TGF-βRII antibody in a cell expressing FAP and TGF-βRII, as measured as reduction in pSMAD2 expression as described in Example 7. In certain embodiments, a bispecific binding moiety of the present disclosure has a potency in blocking TGF-βRII mediated signaling in a range of about 100-500, or in a range of about 100-300, or about 200, fold higher than the potency of a reference anti-TGF-βRII antibody in a cell expressing FAP and TGF-βRII, as measured as reduction in pSMAD2 expression as described in Example 7.

In certain embodiments, the reference anti-TGF-βRII antibody is a bivalent monospecific antibody comprising a heavy chain having an amino acid sequences as set forth in SEQ ID NO: 1 and a light chain having an amino acid sequence as set forth in SEQ ID NO: 2.

In certain embodiments, a bispecific binding moiety of the present disclosure has a higher potency in blocking TGF-βRII-mediated signaling in a cell expressing FAP and TGF-βRII than in a cell expressing TGF-βRII and no, or undetectable levels of FAP.

In certain embodiments, a cell expressing both FAP and TGF-βRII is an A549-FAP cell, such as for instance an A549 parental cell overexpressing human FAP, as described in Example 8. In certain embodiments, an A549-FAP⁺ cell expresses at least in the range of about 1×10⁵-1×10⁶ FAP molecules on the cell surface. In certain embodiments, an A549-FAP⁺ cell expresses at least about 1×10⁶ FAP molecules on the cell surface. In certain embodiments, an A549-FAP⁺ cell expresses at least about 5000-10000 TGF-βRII molecules on the cell surface. In certain embodiments, an A549-FAP⁺ cell expresses at least about 10000 TGF-βRII molecules on the cell surface. In certain embodiments, the levels of FAP and TGF-βRII are measured using quantibrite bead methodology as described in Example 8.

In certain embodiments, a cell expressing TGF-βRII and no FAP, or undetectable levels of FAP is an A549 parental cell as described herein. A549 parental cells are publicly available, for instance from ATCC (cat. no. CCL-185). In certain embodiments, no, or undetectable levels of FAP, refers to less than about 300 FAP molecules present on the cell surface. In certain embodiments, an A549 parental cell expresses less than about 200 FAP molecules on the cell surface. In certain embodiments, A549 parental cell expresses at least about 7000 TGF-βRII molecules on the cell surface. In certain embodiments, the levels of FAP and TGF-βRII are measured using quantibrite bead methodology as described in Example 8.

In certain embodiments, the fold difference of FAP receptors on A549-FAP⁺ cells as compared to A549 parental cells, is at least in the range of 500-5000 fold, in particular in a range of 1000-5000 fold, in particular in the range of 4000-5000 fold. In certain embodiments, the fold difference of FAP receptors on A549-FAP⁺ cells as compared to A549 parental cells, is at least 4500 fold. In certain embodiments, the expression of TGF-βRII receptors on A549-FAP⁺ cells and A549 parental cells is comparable and in the range of 1-2 fold difference.

For the purposes of the present disclosure, determining if a bispecific binding moiety has a higher potency in blocking TGF-βRII-mediated signaling in cells expressing both FAP and TGF-βRII than in cells expressing TGF-βRII and no, or undetectable levels of FAP, is done by using the mixed culture pSMAD2 assay as described in Example 9. Therefore, in certain embodiments, the potency in blocking TGF-βRII-mediated signaling is measured in a mixed culture pSMAD2 assay as described in Example 9.

In brief, the mixed culture pSMAD2 assay as described in Example 9 is performed by using A549 parental cells and A549-FAP⁺ cells cultured in a suitable buffer. Cells are trypsinized, washed and A549-FAP⁺ cells are labeled with CFSE. A549 parental and A549-FAP⁺ cells are then mixed 1:1 and incubated with test antibodies and recombinant human TGF-β1. Washed and fixed cells are then stained for pSMAD2 and acquired by a flow cytometer. The potency in blocking TGF-βRII mediated signaling is determined in IC50 (ug/ml) for A549 parental cells and A549-FAP⁺ cells.

In certain embodiments, a bispecific binding moiety of the present disclosure has a potency in blocking TGF-βRII-mediated signaling in cells expressing both FAP and TGF-βRII of at least about 100 fold, or between about 100-20,000 fold, higher than in cells expressing TGF-βRII and no, or undetectable levels of FAP. In certain embodiments, a bispecific binding moiety of the present disclosure has a potency in blocking TGF-βRII-mediated signaling in cells expressing both FAP and TGF-βRII of at least about 600-700 fold, or at least about 3000-4000 fold, or at least about 18000-20000 fold higher than in cells expressing TGF-βRII and no, or undetectable levels of FAP. In certain embodiments, the potency in blocking TGF-βRII mediated signaling is determined in IC50 (ug/ml) in a mixed culture pSMAD2 assay.

In certain embodiments, determining if a bispecific binding moiety has a higher potency in blocking TGF-βRII-mediated signaling in cells expressing both FAP and TGF-βRII than in cells expressing TGF-βRII and no, or undetectable levels of FAP, is done in an in vivo study by using an NSG mouse model as described in Example 12.

In brief, A549 parental or A549-FAP⁺ cells are inoculated into the flank of NSG mice. After tumors are established, bispecific antibodies are administered. Mice are sacrificed and single cells are obtained from the collected tumors. Cells are stained for IL-11, pSMAD2 and anti-human IgG.

In certain embodiments, a bispecific binding moiety of the present disclosure targets FAP on a particular CAF cell and simultaneously targets TGF-βRII on the same CAF cell. This is referred to as a cis-mode of activity. The bispecific binding moiety, thereby mediates inhibition of TGF-β induced immunomodulation on CAFs. Additionally, the bispecific binding moiety can mediate cytotoxic activity of immune cells directed to CAFs by means of Fc-mediated effector function.

In certain embodiments, a bispecific binding moiety of the present disclosure targets FAP expressed on a CAF cell and simultaneously targets TGF-βRII expressed on another cell. In certain embodiments, a bispecific binding moiety of the present disclosure targets FAP expressed on CAF cells and simultaneously targets TGF-βRII expressed on immune effector cells. This is referred to as a trans-mode of activity, whereby the bispecific binding moiety prevents immune cell inhibitory signaling in TGF-βRII expressing immune effector cells in the tumor microenvironment.

The present disclosure therefore also provides a bispecific binding moiety comprising a FAP binding domain and a TGF-βRII binding domain, wherein the FAP binding domain binds to FAP expressed on a first cell and the TGF-βRII binding domain binds to TGF-βRII expressed on a second cell.

In certain embodiments, upon binding of the FAP binding domain to FAP expressed on the first cell and binding of the TGF-βRII binding domain to TGF-βRII expressed on the second cell, the TGF-βRII binding domain blocks TGF-βRII mediated signaling in the second cell.

In certain embodiments, the first and the second cell are different types of cells. In certain embodiments, the first cell is a fibroblast cell. In certain embodiments, the second cell is a non-fibroblast cell. In certain embodiments the second cell is an immune effector cell or a tumor cell. In certain embodiments, an immune effector cell is an NK cell, a T cell, a B cell, a monocyte, a macrophage, a dendritic cell or a neutrophilic granulocyte.

In certain embodiments, the blocking of TGF-βRII mediated signaling in the second cell, according to the trans-mode of activity, is measured in a TGF-βRII reporter assay as described in Example 15.

In brief, the TGF-βRII reporter assay as described in Example 15 is performed by using recombinant human TGF-β1 binding to TGF-βRII expressed on HEK-Blue-TGF-βRII reporter cells and MRC-5 cells. Bispecific binding moieties of the present disclosure are added and the disruption of TGF-βRII binding to its ligand is measured by detecting secreted alkaline phosphatase (SEAP) levels using a suitable substrate, such as for instance QUANTI-Blue™ substrate.

In certain embodiments, a bispecific binding moiety of the present disclosure has a higher activity in reducing tumor volume than a reference anti-TGF-βRII antibody. In certain embodiments, the reference antibody is a bivalent monospecific antibody targeting TGF-βRII comprising a heavy chain having an amino acid sequences as set forth in SEQ ID NO: 1 and a light chain having an amino acid sequence as set forth in SEQ ID NO: 2.

The present disclosure therefore also provides a bispecific binding moiety comprising a FAP binding domain and a TGF-βRII binding domain, wherein the bispecific binding moiety has a higher activity in reducing tumor volume than a reference anti-TGF-βRII antibody. In certain embodiments, the bispecific binding moiety is dosed with a two-fold lower to up to twenty-fold lower number of TGF-βRII binding domains than the bivalent monospecific anti-TGF-βRII reference antibody. For example, a bispecific binding moiety, which is monovalent for binding to FAP and monovalent for binding to TGF-βRII, when dosed at 3 mg/kg has a higher activity in reducing tumor volume than a reference antibody, which is bivalent for binding to TGF-βRII and which is dosed at 30 mg/kg. Also, a bispecific binding moiety, which is monovalent for binding to FAP and monovalent for binding to TGF-βRII, when dosed at 30 mg/kg has a higher activity in reducing tumor volume than a reference antibody, which is bivalent for binding to TGF-βRII and which is dosed at 30 mg/kg.

In certain embodiments, the activity in reducing tumor volume is determined by measuring tumor volume reduction in an in vivo mouse study, in particular in an in vivo mouse study using A549-FAP⁺ cells transplanted in a BALB/c nu/nu mice, as described in Example 13.

In certain embodiments, a bispecific binding moiety of the present disclosure has a tumor volume reduction that is at least 1.5 fold, or between 1.5-2 fold, of the tumor volume reduction of a reference antibody.

In certain embodiments, the reference antibody is a bivalent monospecific antibody targeting TGF-βRII comprising a heavy chain having an amino acid sequence as set forth in SEQ ID NO: 1 and a light chain having an amino acid sequence as set forth in SEQ ID NO: 2.

In certain embodiments, a bispecific binding moiety of the present disclosure reduces tumor volume in an in vivo mouse model, compared to untreated mice.

In certain embodiments, a bispecific binding moiety of the present disclosure reduces tumor volume when administered as a single agent.

The present disclosure therefore also provides a bispecific binding moiety comprising a FAP binding domain and a TGF-βRII binding domain, wherein the bispecific binding moiety induces tumor volume reduction as a single agent.

In certain embodiments, the FAP binding domain of a bispecific binding moiety of the present disclosure comprises a heavy chain variable region comprising:
a) heavy chain CDR1 (HCDR1), heavy chain CDR2 (HCDR2), and heavy chain CDR3 (HCDR3), having an amino acid sequence as set forth in SEQ ID NO: 12, SEQ ID NO: 13, and SEQ ID NO: 14, respectively;
b) heavy chain CDR1 (HCDR1), heavy chain CDR2 (HCDR2), and heavy chain CDR3 (HCDR3), having an amino acid sequence as set forth in SEQ ID NO: 16, SEQ ID NO: 17, and SEQ ID NO: 18, respectively; or
c) heavy chain CDR1 (HCDR1), heavy chain CDR2 (HCDR2), and heavy chain CDR3 (HCDR3), having an amino acid sequence as set forth in SEQ ID NO: 20, SEQ ID NO: 21, and SEQ ID NO: 22, respectively.

The heavy chain variable regions of the FAP binding domains of a bispecific binding moiety of the present disclosure may comprise a limited number, such as for instance one, two, three, four, five, six, seven, eight, nine, or ten, non-conservative amino acid substitutions, or an unlimited number of conservative amino acid substitutions.

In certain embodiments, the FAP binding domain of a bispecific binding moiety of the present disclosure also includes FAP binding domain variants thereof, wherein each of the HCDR1 or HCDR2 may comprise at most three, two, or one amino acid variations. In certain embodiments, only one of the HCDR1 or HCDR2 may comprise at most three, two, or one non-conservative amino acid variations. In certain embodiments, such variants do not comprise amino acid variations in HCDR3. In certain embodiments, the amino acid variation is a conservative amino acid substitution.

In certain embodiments, a FAP binding domain of a bispecific binding moiety of the present disclosure comprises a heavy chain variable region having an amino acid sequence as set forth in any one of SEQ ID NOs: 11, 15 or 19, or a variant thereof. In certain embodiments, a FAP binding domain of a bispecific binding moiety of the present disclosure comprises a heavy chain variable region having an amino acid sequence as set forth in any one of SEQ ID NOs: 11, 15 or 19, or a variant having at least 80%, at least 85%, at least 90%, or at least 95% sequence identity thereto.

In certain embodiments, a FAP binding domain of a bispecific binding moiety of the present disclosure also comprises FAP binding domain variants, which, in addition to the variations in the HCDR1 and HCDR2 referred to above, comprise one or more variations in the framework regions. A variation can be any type of amino acid variation described herein, such as for instance a conservative amino acid substitution or non-conservative amino acid substitution resulting from somatic hypermutation or affinity maturation. In certain embodiments, a FAP binding domain variant of a bispecific binding moiety of the present disclosure comprises no variations in the CDR regions but comprises one or more variations in the framework regions. Such variants have at least 80%, at least 85%, at least 90%, or at least 95% sequence identity to the sequences disclosed herein, and are expected to retain FAP binding specificity. Thus, in certain embodiments, a FAP binding domain of a bispecific binding moiety of the present disclosure comprises:
a heavy chain variable region having at least 80%, at least 85%, at least 90%, or at least 95% sequence identity to the amino acid sequence as set forth in SEQ ID NO: 11, which heavy chain variable region comprises a HCDR1 amino acid sequence as set forth in SEQ ID NO: 12; a HCDR2 amino acid sequence as set forth in SEQ ID NO: 13; and a HCDR3 amino acid sequence as set forth in SEQ ID NO: 14;
a heavy chain variable region having at least 80%, at least 85%, at least 90%, or at least 95% sequence identity to the amino acid sequence as set forth in SEQ ID NO: 15, which heavy chain variable region comprises a HCDR1 amino acid sequence as set forth in SEQ ID NO: 16; a HCDR2 amino acid sequence as set forth in SEQ ID NO: 17; and a HCDR3 amino acid sequence as set forth in SEQ ID NO: 18; or
a heavy chain variable region having at least 80%, at least 85%, at least 90%, or at least 95% sequence identity to the amino acid sequence as set forth in SEQ ID NO: 19, which heavy chain variable region comprises a HCDR1 amino acid sequence as set forth in SEQ ID NO: 20; a HCDR2 amino acid sequence as set forth in SEQ ID NO: 21; and a HCDR3 amino acid sequence as set forth in SEQ ID NO: 22.

The polypeptides of the present disclosure and the binding domains of the bispecific binding moiety of the present disclosure have been generated with a common light chain, in particular with a common light chain referred to as VK1-39/JK1. The polypeptides of the present disclosure and the binding domains of the bispecific binding moiety of the present disclosure can comprise any suitable light chain, including but not limited to common light chains known in the art. In certain embodiments, the polypeptides of the present disclosure and the binding domains of the bispecific binding moiety of the present disclosure comprise common light chain VK1-39/JK1, or a variant thereof harboring a limited number, such as for instance one, two, or three, non-conservative amino acid substitutions, or an unlimited number of conservative amino acid substitutions.

In certain embodiments, a FAP binding domain of a bispecific binding moiety of the present disclosure comprises a light chain variable region having an amino acid sequence as set forth in SEQ ID NO: 52, or a variant thereof. In certain embodiments, a FAP binding domain of a bispecific binding moiety of the present disclosure comprises a light chain variable region having an amino acid sequence as set forth in SEQ ID NO: 52, or a variant having at least 80%, at least 85%, at least 90%, or at least 95% sequence identity thereto.

In certain embodiments, a FAP binding domain of a bispecific binding moiety of the present disclosure comprises a light chain variable region comprising light chain CDR1 (LCDR1), light chain CDR2 (LCDR2), and light chain CDR3 (LCDR3), having an amino acid sequence as set forth in SEQ ID NO: 53, SEQ ID NO: 54, and SEQ ID NO: 55. In certain embodiments, the light chain variable region of a FAP binding domain of a bispecific binding moiety of the present disclosure also includes variants thereof, wherein each of the LCDRs may comprise at most three, two, or one amino acid variations. In certain embodiments, the amino acid variation is a conservative amino acid substitution.

In certain embodiments, a FAP binding domain of a bispecific binding moiety of the present disclosure also includes FAP binding domain variants, which, in addition to the variations in the LCDRs referred to above, comprise one or more variations in the framework regions. A variation is preferably a conservative amino acid substitution. In certain embodiments, a FAP binding domain variant of a bispecific binding moiety of the present disclosure comprises no variations in the LCDR regions but comprises one or more variations in the framework regions. Such variants have at least 80%, at least 85%, at least 90%, or at least 95% sequence identity to the sequences disclosed herein. Thus, in certain embodiments, a FAP binding domain of a bispecific binding moiety of the present disclosure comprises:
a light chain variable region having at least 80%, at least 85%, at least 90%, or at least 95% sequence identity to the amino acid sequence as set forth in SEQ ID NO: 52, which light chain variable region comprises a LCDR1 amino acid sequence as set forth in SEQ ID NO: 53; a LCDR2 amino acid sequence as set forth in SEQ ID NO: 54; and a LCDR3 amino acid sequence as set forth in SEQ ID NO: 55.

A light chain or light chain variable region comprising these LCDRs and/or light chain variable region can be, for example, the light chain referred to in the art as VK1-39/JK1. This is a common light chain. The term ‘common light chain’ according to the present disclosure refers to a light chain that is capable of pairing with multiple different heavy chains, such as for instance heavy chains having different antigen or epitope binding specificities. A common light chain is particularly useful in the generation of, for instance, bispecific or multispecific antibodies, where antibody production is more efficient when all binding domains comprise the same light chain. The term “common light chain” encompasses light chains that are identical or have some amino acid sequence differences while the binding specificity of the full length antibody is not affected. It is for instance possible within the scope of the definition of common light chains as used herein, to prepare or find light chains that are not identical but still functionally equivalent, e.g., by using well established variations that introduce conservative amino acid changes, changes of amino acids in regions that are known to or are shown to not or only partly contribute to binding specificity when paired with the heavy chain, and the like.

Apart from a common light chain comprising the LCDRs and/or light chain variable region referred to above, other common light chains known in the art may be used. Examples of such common light chains include, but are not limited to: VK1-39/JK5, comprising a light chain variable region comprising a light chain CDR1 (LCDR1), light chain CDR2 (LCDR2), and light chain CDR3 (LCDR3), of a light chain variable region having an amino acid sequence as set forth in SEQ ID NO: 56. In certain embodiments, the light chain comprises a light chain variable region comprising a light chain CDR1 (LCDR1), light chain CDR2 (LCDR2), and light chain CDR3 (LCDR3), of a light chain variable region having an amino acid sequence as set forth in SEQ ID NO: 56, wherein each of the LCDRs may comprise at most three, two, or one amino acid variations, for example substitutions. In certain embodiments, the light chain comprises a light chain variable region having an amino acid sequence as set forth in SEQ ID NO: 56, or having at least 80%, at least 85%, at least 90%, or at least 95% sequence identity thereto. In certain embodiments, the light chain comprises a light chain CDR1 (LCDR1), light chain CDR2 (LCDR2), and light chain CDR3 (LCDR3) having an amino acid sequence as set forth in SEQ ID NO: 53, SEQ ID NO: 54, and SEQ ID NO: 59; VK3-15/JK1, comprising a light chain variable region comprising a light chain CDR1 (LCDR1), light chain CDR2 (LCDR2), and light chain CDR3 (LCDR3), of a light chain variable region having an amino acid sequence as set forth in SEQ ID NO: 61. In certain embodiments, the light chain comprises a light chain variable region comprising a light chain CDR1 (LCDR1), light chain CDR2 (LCDR2), and light chain CDR3 (LCDR3), of a light chain variable region having an amino acid sequence as set forth in SEQ ID NO: 61, wherein each of the LCDRs may comprise at most three, two, or one amino acid variations, for example substitutions. In certain embodiments, the light chain comprises a light chain variable region having an amino acid sequence as set forth in SEQ ID NO: 61, or having at least 80%, at least 85%, at least 90%, or at least 95% sequence identity thereto. In certain embodiments, the light chain comprises a light chain CDR1 (LCDR1), light chain CDR2 (LCDR2), and light chain CDR3 (LCDR3) having an amino acid sequence as set forth in SEQ ID NO: 62, SEQ ID NO: 63, and SEQ ID NO: 64; VK3-20/JK1, comprising a light chain variable region comprising a light chain CDR1 (LCDR1), light chain CDR2 (LCDR2), and light chain CDR3 (LCDR3), of a light chain variable region having an amino acid sequence as set forth in SEQ ID NO: 66. In certain embodiments, the light chain comprises a light chain variable region comprising a light chain CDR1 (LCDR1), light chain CDR2 (LCDR2), and light chain CDR3 (LCDR3), of a light chain variable region having an amino acid sequence as set forth in SEQ ID NO: 66, wherein each of the LCDRs may comprise at most three, two, or one amino acid variations, for example substitutions. In certain embodiments, the light chain comprises a light chain variable region having an amino acid sequence as set forth in SEQ ID NO: 66, or having at least 80%, at least 85%, at least 90%, or at least 95% sequence identity thereto. In certain embodiments, the light chain comprises a light chain CDR1 (LCDR1), light chain CDR2 (LCDR2), and light chain CDR3 (LCDR3) having an amino acid sequence as set forth in SEQ ID NO: 67, SEQ ID NO: 63, and SEQ ID NO: 68; and VL3-21/JL3, comprising a light chain variable region comprising a light chain CDR1 (LCDR1), light chain CDR2 (LCDR2), and light chain CDR3 (LCDR3), of a light chain variable region having an amino acid sequence as set forth in SEQ ID NO: 32. In certain embodiments, the light chain comprises a light chain variable region comprising a light chain CDR1 (LCDR1), light chain CDR2 (LCDR2), and light chain CDR3 (LCDR3), of a light chain variable region having an amino acid sequence as set forth in SEQ ID NO: 32, wherein each of the LCDRs may comprise at most three, two, or one amino acid variations, for example substitutions. In certain embodiments, the light chain comprises a light chain variable region having an amino acid sequence as set forth in SEQ ID NO: 32, or having at least 80%, at least 85%, at least 90%, or at least 95% sequence identity thereto. In certain embodiments, the light chain comprises a light chain CDR1 (LCDR1), light chain CDR2 (LCDR2), and light chain CDR3 (LCDR3) having an amino acid sequence as set forth in SEQ ID NO: 36, SEQ ID NO: 38, and SEQ ID NO: 57.

VK1-39 is short for Immunoglobulin Variable Kappa 1-39 Gene. The gene is also known as Immunoglobulin Kappa Variable 1-39; IGKV139; IGKV1-39; IgVκ1-39. External Ids for the gene are HGNC: 5740; Entrez Gene: 28930; Ensembl: ENSG00000242371. An amino acid sequence for VK1-39 is given as SEQ ID NO: 60. This is the sequence of the V-region. The V-region can be combined with one of five J-regions. Suitable VJ-region sequences are indicated as VK1-39/JK1 (SEQ ID NO: 52) and VK1-39/JK5 (SEQ ID NO: 56); alternative names are IgVκ1-39*01/IGJκ1*01 or IgVκ1-39*01/IGJκ5*01 (nomenclature according to the IMGT database worldwide web at imgt.org). These names are exemplary and encompass allelic variants of the gene segments.

VK3-15 is short for Immunoglobulin Variable Kappa 3-15 Gene. The gene is also known as Immunoglobulin Kappa Variable 3-15; IGKV315; IGKV3-15; IgVκ3-15. External Ids for the gene are HGNC: 5816; Entrez Gene: 28913; Ensembl: ENSG00000244437. An amino acid sequence for VK3-15 is given as SEQ ID NO: 65. This is the sequence of the V-region. The V-region can be combined with one of five J-regions. A suitable VJ-region sequence is indicated as VK3-15/JK1 (SEQ ID NO: 61); alternative name is Vκ3-15*01/IGJκ1*01 (nomenclature according to the IMGT database worldwide web at imgt.org). This name is exemplary and encompasses allelic variants of the gene segments.

VK3-20 is short for Immunoglobulin Variable Kappa 3-20 Gene. The gene is also known as Immunoglobulin Kappa Variable 3-20; IGKV320; IGKV3-20; IgVκ3-20. External Ids for the gene are HGNC: 5817; Entrez Gene: 28912; Ensembl: ENSG00000239951. An amino acid sequence for VK3-20 is indicated as SEQ ID NO: 28. This is the sequence of the V-region. The V-region can be combined with one of five J-regions. A suitable VJ-region sequence is indicated as VK3-20/JK1 (SEQ ID NO: 66); alternative name is IgVκ3-20*01/IGJκ1*01 (nomenclature according to the IMGT database worldwide web at imgt.org). This name is exemplary and encompasses allelic variants of the gene segments.

VL3-21 is short for Immunoglobulin Variable Lambda 3-21 Gene. The gene is also known as Immunoglobulin Lambda Variable 3-21; IGLV321; IGLV3-21; IgVλ3-21. External Ids for the gene are HGNC: 5905; Entrez Gene: 28796; Ensembl: ENSG00000211662. An amino acid sequence for VL3-21 is given as SEQ ID NO: 58. This is the sequence of the V-region. The V-region can be combined with one of five J-regions. A suitable VJ-region sequence is indicated as VL3-21/JL3 (SEQ ID NO: 32); alternative name is IgVλ3-21/IGJλ3 (nomenclature according to the IMGT database worldwide web at imgt.org). This name is exemplary and encompasses allelic variants of the gene segments.

Further, any light chain variable region of a FAP antibody available in the art may be used, as may any other light chain variable region that can readily be obtained, such as from, for instance, an antibody display library by showing antigen binding activity when paired with a FAP binding domain of a bispecific binding moiety of the present disclosure.

In certain embodiments, a FAP binding domain of a bispecific binding moiety of the present disclosure may further comprise a CH1 and CL region. Any CH1 domain may be used, in particular a human CH1 domain. An example of a suitable CH1 domain is provided by the amino acid sequence provided as SEQ ID NO: 39. Any CL domain may be used, in particular a human CL. An example of a suitable CL domain is provided by the amino acid sequence provided as SEQ ID NO: 51.

In certain embodiments, the TGF-βRII binding domain of a bispecific binding moiety of the present disclosure which blocks TGF-βRII binding to TGF-βRII ligand is a TGF-βRII binding moiety as described in WO 2021/133167, in particular on page 54, line 11, to page 55, line 1; page 59, line 3, to page 60, line 5, and in FIG. 6. In certain embodiments, the TGF-βRII binding domain of a bispecific binding moiety of the present disclosure, which blocks TGF-βRII binding to TGF-βRII ligand, is described in WO 2021/133167 as one of SEQ ID NO: 10-12; SEQ ID NO: 22-91, and SEQ ID NO: 93-96.

In certain embodiments, the TGF-βRII binding domain of a bispecific binding moiety of the present disclosure comprises a heavy chain variable region comprising:
a) heavy chain CDR1 (HCDR1), heavy chain CDR2 (HCDR2), and heavy chain CDR3 (HCDR3), having an amino acid sequence as set forth in SEQ ID NO: 24, SEQ ID NO: 25, and SEQ ID NO: 26, respectively;
b) heavy chain CDR1 (HCDR1), heavy chain CDR2 (HCDR2), and heavy chain CDR3 (HCDR3), having an amino acid sequence as set forth in SEQ ID NO: 24, SEQ ID NO: 29, and SEQ ID NO: 30, respectively;
c) heavy chain CDR1 (HCDR1), heavy chain CDR2 (HCDR2), and heavy chain CDR3 (HCDR3), having an amino acid sequence as set forth in SEQ ID NO: 24, SEQ ID NO: 33, and SEQ ID NO: 34, respectively; or
d) heavy chain CDR1 (HCDR1), heavy chain CDR2 (HCDR2), and heavy chain CDR3 (HCDR3), having an amino acid sequence as set forth in SEQ ID NO: 24, SEQ ID NO: 37, and SEQ ID NO: 34, respectively.

The heavy chain variable regions of the TGF-βRII binding domains of a bispecific binding moiety of the present disclosure may comprise a limited number, such as for instance one, two, or three, non-conservative amino acid substitutions, or an unlimited number of conservative amino acid substitutions.

In certain embodiments, the TGF-βRII binding domain of a bispecific binding moiety of the present disclosure also includes TGF-βRII binding domain variants thereof, wherein each of the HCDR1 or HCDR2 may comprise at most three, two, or one amino acid variations. In certain embodiments, only one of the HCDR1 or HCDR2 may comprise at most three, two, or one amino acid variations. In certain embodiments, such variants do not comprise amino acid variations in HCDR3. In certain embodiments, the amino acid variation is a conservative amino acid substitution. A conservative amino acid substitution is as described further herein.

In certain embodiments, a TGF-βRII binding domain of a bispecific binding moiety of the present disclosure comprises a heavy chain variable region having an amino acid sequence as set forth in any one of SEQ ID NOs: 23, 27, 31 or 35, or a variant thereof. In certain embodiments, a TGF-βRII binding domain of a bispecific binding moiety of the present disclosure comprises a heavy chain variable region having an amino acid sequence as set forth in any one of SEQ ID NOs: 23, 27, 31 or 35, or having at least 80%, at least 85%, at least 90%, or at least 95% sequence identity thereto.

In certain embodiments, a TGF-βRII binding domain of a bispecific binding moiety of the present disclosure also includes TGF-βRII binding domain variants, which, in addition to the variations in the HCDR1 and HCDR2 referred to above, comprise one or more variations in the framework regions. A variation can be any type of amino acid variation described herein, such as for instance a conservative amino acid substitution or non-conservative amino acid substitution resulting from somatic hypermutation or affinity maturation. In certain embodiments, a TGF-βRII binding domain variant of a bispecific binding moiety of the present disclosure comprises no variations in the CDR regions but comprises one or more variations in the framework regions. Such variants have at least 80%, at least 85%, at least 90%, or at least 95% sequence identity to the sequences disclosed herein, and are expected to retain TGF-βRII binding specificity. Thus, in certain embodiments, a TGF-βRII binding domain of a bispecific binding moiety of the present disclosure comprises:
a heavy chain variable region having at least 80%, at least 85%, at least 90%, or at least 95% sequence identity to the amino acid sequence as set forth in SEQ ID NO: 23, which heavy chain variable region comprises a HCDR1 amino acid sequence as set forth in SEQ ID NO: 24; a HCDR2 amino acid sequence as set forth in SEQ ID NO: 25; and a HCDR3 amino acid sequence as set forth in SEQ ID NO: 26;
a heavy chain variable region having at least 80%, at least 85%, at least 90%, or at least 95% sequence identity to the amino acid sequence as set forth in SEQ ID NO: 27, which heavy chain variable region comprises a HCDR1 amino acid sequence as set forth in SEQ ID NO: 24; a HCDR2 amino acid sequence as set forth in SEQ ID NO: 29; and a HCDR3 amino acid sequence as set forth in SEQ ID NO: 30;
a heavy chain variable region having at least 80%, at least 85%, at least 90%, or at least 95% sequence identity to the amino acid sequence as set forth in SEQ ID NO: 31, which heavy chain variable region comprises a HCDR1 amino acid sequence as set forth in SEQ ID NO: 24; a HCDR2 amino acid sequence as set forth in SEQ ID NO: 33; and a HCDR3 amino acid sequence as set forth in SEQ ID NO: 34; or
a heavy chain variable region having at least 80%, at least 85%, at least 90%, or most at least 95% sequence identity to the amino acid sequence as set forth in SEQ ID NO: 35, which heavy chain variable region comprises a HCDR1 amino acid sequence as set forth in SEQ ID NO: 24; a HCDR2 amino acid sequence as set forth in SEQ ID NO: 37; and a HCDR3 amino acid sequence as set forth in SEQ ID NO: 34.

Any light chain variable region of a TGF-βRII antibody available in the art may be used, for example as described herein, as may any other light chain variable region that can readily be obtained, such as from, for instance, an antibody display library by showing antigen binding activity when paired with a TGF-βRII binding domain of a bispecific binding moiety of the present disclosure. In certain embodiments, the TGF-βRII binding domain of a bispecific binding moiety of the present disclosure comprises the same or substantially the same light chain as the FAP binding domain.

In certain embodiments, the TGF-βRII binding domain of a bispecific binding moiety of the present disclosure comprises a light chain variable region having an amino acid sequence as set forth in SEQ ID NO: 52, or a variant thereof. In certain embodiments, the TGF-βRII binding domain of a bispecific binding moiety of the present disclosure comprises a light chain variable region having an amino acid sequence as set forth in SEQ ID NO: 52, or a variant having at least 80%, at least 85%, at least 90%, or at least 95% sequence identity thereto.

In certain embodiments, the TGF-βRII binding domain of a bispecific binding moiety of the present disclosure comprises a light chain variable region comprising light chain CDR1 (LCDR1), light chain CDR2 (LCDR2), and light chain CDR3 (LCDR3), having an amino acid sequence as set forth in SEQ ID NO: 53, SEQ ID NO: 54, and SEQ ID NO: 55. In certain embodiments, the light chain variable region of a TGF-βRII binding domain of a bispecific binding moiety of the present disclosure also includes variants thereof, wherein each of the LCDRs may comprise at most three, two, or one conservative or non-conservative amino acid variations. In certain embodiments, the amino acid variation is a conservative amino acid substitution.

In certain embodiments, a TGF-βRII binding domain of a bispecific binding moiety of the present disclosure may further comprise a CH1 and CL region. Any CH1 domain may be used, in particular a human CH1 domain. An example of a suitable CH1 domain is provided by the amino acid sequence provided as SEQ ID NO: 39. Any CL domain may be used, in particular a human CL. An example of a suitable CL domain is provided by the amino acid sequence provided as SEQ ID NO: 51.

The present invention thus also provides a bispecific binding moiety comprising a FAP binding domain and a TGF-βRII binding domain, wherein the FAP binding domain comprises a heavy chain variable region, and optionally a light chain variable region and CH1 and CL regions, as described herein. In certain embodiments, the bispecific binding moiety further comprises a TGF-βRII binding domain that comprises a heavy chain variable region, and optionally a light chain variable region and CH1 and CL regions, as described herein.

The present invention thus also provides a bispecific binding moiety comprising a FAP binding domain and a TGF-βRII binding domain, wherein the TGF-βRII binding domain comprises a heavy chain variable region, and optionally a light chain variable region and CH1 and CL regions, as described herein. In certain embodiments, the bispecific binding moiety further comprises a FAP binding domain that comprises a heavy chain variable region, and optionally a light chain variable region and CH1 and CL regions, as described herein.

In certain embodiments, any FAP binding domain disclosed herein can be combined with any TGF-βRII binding domain disclosed herein to produce a bispecific binding moiety of the present disclosure. The present disclosure thus provides exemplary bispecific binding moieties PB1-PB12, as presented in Table 2.

In one embodiment, the present disclosure provides a bispecific binding moiety comprising:
a FAP binding domain as described herein comprising a heavy chain CDR1 (HCDR1), heavy chain CDR2 (HCDR2), and heavy chain CDR3 (HCDR3), having an amino acid sequence as set forth in SEQ ID NO: 12, SEQ ID NO: 13, and SEQ ID NO: 14, respectively; and
a TGF-βRII binding domain as described herein comprising a heavy chain CDR1 (HCDR1), heavy chain CDR2 (HCDR2), and heavy chain CDR3 (HCDR3), having an amino acid sequence as set forth in SEQ ID NO: 24, SEQ ID NO: 25, and SEQ ID NO: 26, respectively,
wherein each of the HCDR1 and HCDR2 may comprise at most three, two, or one amino acid variations, for example substitutions. In certain embodiments, the HCDR3 do not comprise amino acid variations. In certain embodiments, the HCDRs do not comprise amino acid variations.

In one embodiment, the present disclosure provides a bispecific binding moiety comprising:
a FAP binding domain as described herein comprising a heavy chain CDR1 (HCDR1), heavy chain CDR2 (HCDR2), and heavy chain CDR3 (HCDR3), having an amino acid sequence as set forth in SEQ ID NO: 16, SEQ ID NO: 17, and SEQ ID NO: 18, respectively; and
a TGF-βRII binding domain as described herein comprising a heavy chain CDR1 (HCDR1), heavy chain CDR2 (HCDR2), and heavy chain CDR3 (HCDR3), having an amino acid sequence as set forth in SEQ ID NO: 24, SEQ ID NO: 33, and SEQ ID NO: 34, respectively,
wherein each of the HCDR1 and HCDR2 may comprise at most three, two, or one amino acid variations, for example substitutions. In certain embodiments, the HCDR3 do not comprise amino acid variations. In certain embodiments, the HCDRs do not comprise amino acid variations.

In one embodiment, the present disclosure provides a bispecific binding moiety comprising:
a FAP binding domain as described herein comprising a heavy chain CDR1 (HCDR1), heavy chain CDR2 (HCDR2), and heavy chain CDR3 (HCDR3), having an amino acid sequence as set forth in SEQ ID NO: 20, SEQ ID NO: 21, and SEQ ID NO: 22, respectively; and
a TGF-βRII binding domain as described herein comprising a heavy chain CDR1 (HCDR1), heavy chain CDR2 (HCDR2), and heavy chain CDR3 (HCDR3), having an amino acid sequence as set forth in SEQ ID NO: 24, SEQ ID NO: 29, and SEQ ID NO: 30, respectively,
wherein each of the HCDR1 and HCDR2 may comprise at most three, two, or one amino acid variations, for example substitutions. In certain embodiments, the HCDR3 do not comprise amino acid variations. In certain embodiments, the HCDRs do not comprise amino acid variations.

In one embodiment, the present disclosure provides a bispecific binding moiety comprising:
a FAP binding domain as described herein comprising a heavy chain CDR1 (HCDR1), heavy chain CDR2 (HCDR2), and heavy chain CDR3 (HCDR3), having an amino acid sequence as set forth in SEQ ID NO: 16, SEQ ID NO: 17, and SEQ ID NO: 18, respectively; and
a TGF-βRII binding domain as described herein comprising a heavy chain CDR1 (HCDR1), heavy chain CDR2 (HCDR2), and heavy chain CDR3 (HCDR3), having an amino acid sequence as set forth in SEQ ID NO: 24, SEQ ID NO: 37, and SEQ ID NO: 34, respectively,
wherein each of the HCDR1 and HCDR2 may comprise at most three, two, or one amino acid variations, for example substitutions. In certain embodiments, the HCDR3 do not comprise amino acid variations. In certain embodiments, the HCDRs do not comprise amino acid variations.

In one embodiment, the present disclosure provides a bispecific binding moiety comprising:
a FAP binding domain as described herein comprising a heavy chain CDR1 (HCDR1), heavy chain CDR2 (HCDR2), and heavy chain CDR3 (HCDR3), having an amino acid sequence as set forth in SEQ ID NO: 12, SEQ ID NO: 13, and SEQ ID NO: 14, respectively; and
a TGF-βRII binding domain as described herein comprising a heavy chain CDR1 (HCDR1), heavy chain CDR2 (HCDR2), and heavy chain CDR3 (HCDR3), having an amino acid sequence as set forth in SEQ ID NO: 24, SEQ ID NO: 25, and SEQ ID NO: 26, respectively,
wherein the FAP binding domain and TGF-βRII binding domain comprise a light chain CDR1 (LCDR1) having an amino acid sequence as set forth in SEQ ID NO: 53, light chain CDR2 (LCDR2) having an amino acid sequence as set forth in SEQ ID NO: 54, and light chain CDR3 (LCDR3) having an amino acid sequence as set forth in SEQ ID NO: 55, and
wherein each of the HCDR1, HCDR2, LCDR1, LCDR2, and LCDR3 may comprise at most three, two, or one amino acid variations, for example substitutions. In certain embodiments, the HCDR3 do not comprise amino acid variations. In certain embodiments, the HCDRs and/or LCDRs do not comprise amino acid variations.

In one embodiment, the present disclosure provides a bispecific binding moiety comprising:
a FAP binding domain as described herein comprising a heavy chain CDR1 (HCDR1), heavy chain CDR2 (HCDR2), and heavy chain CDR3 (HCDR3), having an amino acid sequence as set forth in SEQ ID NO: 16, SEQ ID NO: 17, and SEQ ID NO: 18, respectively; and
a TGF-βRII binding domain as described herein comprising a heavy chain CDR1 (HCDR1), heavy chain CDR2 (HCDR2), and heavy chain CDR3 (HCDR3), having an amino acid sequence as set forth in SEQ ID NO: 24, SEQ ID NO: 33, and SEQ ID NO: 34, respectively,
wherein the FAP binding domain and TGF-βRII binding domain comprise a light chain CDR1 (LCDR1) having an amino acid sequence as set forth in SEQ ID NO: 53, light chain CDR2 (LCDR2) having an amino acid sequence as set forth in SEQ ID NO: 54, and light chain CDR3 (LCDR3) having an amino acid sequence as set forth in SEQ ID NO: 55, and
wherein each of the HCDR1, HCDR2, LCDR1, LCDR2, and LCDR3 may comprise at most three, two, or one amino acid variations, for example substitutions. In certain embodiments, the HCDR3 do not comprise amino acid variations. In certain embodiments, the HCDRs and/or LCDRs do not comprise amino acid variations.

In one embodiment, the present disclosure provides a bispecific binding moiety comprising:
a FAP binding domain as described herein comprising a heavy chain CDR1 (HCDR1), heavy chain CDR2 (HCDR2), and heavy chain CDR3 (HCDR3), having an amino acid sequence as set forth in SEQ ID NO: 20, SEQ ID NO: 21, and SEQ ID NO: 22, respectively; and
a TGF-βRII binding domain as described herein comprising a heavy chain CDR1 (HCDR1), heavy chain CDR2 (HCDR2), and heavy chain CDR3 (HCDR3), having an amino acid sequence as set forth in SEQ ID NO: 24, SEQ ID NO: 29, and SEQ ID NO: 30, respectively,
wherein the FAP binding domain and TGF-βRII binding domain comprise a light chain CDR1 (LCDR1) having an amino acid sequence as set forth in SEQ ID NO: 53, light chain CDR2 (LCDR2) having an amino acid sequence as set forth in SEQ ID NO: 54, and light chain CDR3 (LCDR3) having an amino acid sequence as set forth in SEQ ID NO: 55, and
wherein each of the HCDR1, HCDR2, LCDR1, LCDR2, and LCDR3 may comprise at most three, two, or one amino acid variations, for example substitutions. In certain embodiments, the HCDR3 do not comprise amino acid variations. In certain embodiments, the HCDRs and/or LCDRs do not comprise amino acid variations.

In one embodiment, the present disclosure provides a bispecific binding moiety comprising:
a FAP binding domain as described herein comprising a heavy chain CDR1 (HCDR1), heavy chain CDR2 (HCDR2), and heavy chain CDR3 (HCDR3), having an amino acid sequence as set forth in SEQ ID NO: 16, SEQ ID NO: 17, and SEQ ID NO: 18, respectively; and
a TGF-βRII binding domain as described herein comprising a heavy chain CDR1 (HCDR1), heavy chain CDR2 (HCDR2), and heavy chain CDR3 (HCDR3), having an amino acid sequence as set forth in SEQ ID NO: 24, SEQ ID NO: 37, and SEQ ID NO: 34, respectively,
wherein the FAP binding domain and TGF-βRII binding domain comprise a light chain CDR1 (LCDR1) having an amino acid sequence as set forth in SEQ ID NO: 53, light chain CDR2 (LCDR2) having an amino acid sequence as set forth in SEQ ID NO: 54, and light chain CDR3 (LCDR3) having an amino acid sequence as set forth in SEQ ID NO: 55, and
wherein each of the HCDR1, HCDR2, LCDR1, LCDR2, and LCDR3 may comprise at most three, two, or one amino acid variations, for example substitutions. In certain embodiments, the HCDR3 do not comprise amino acid variations. In certain embodiments, the HCDRs and/or LCDRs do not comprise amino acid variations.

In one embodiment, the present disclosure provides a bispecific binding moiety comprising:
a FAP binding domain as described herein comprising a heavy chain variable region having an amino acid sequence as set forth in SEQ ID NO: 11, or a heavy chain variable region that is at least 80%, at least 85%, at least 90%, or at least 95% sequence identity thereto; and
a TGF-βRII binding domain as described herein comprising a heavy chain variable region having an amino acid sequence as set forth in SEQ ID NO: 23, or a heavy chain variable region that is at least 80%, at least 85%, at least 90%, or at least 95% sequence identity thereto. In certain embodiments, each of the heavy chain variable regions comprise HCDRs that do not comprise amino acid variations. In certain embodiments, each of the heavy chain variable regions do not comprise amino acid variations.

In one embodiment, the present disclosure provides a bispecific binding moiety comprising:
a FAP binding domain as described herein comprising a heavy chain variable region having an amino acid sequence as set forth in SEQ ID NO: 15, or a heavy chain variable region that is at least 80%, at least 85%, at least 90%, or at least 95% sequence identity thereto; and
a TGF-βRII binding domain as described herein comprising a heavy chain variable region having an amino acid sequence as set forth in SEQ ID NO: 31 or a heavy chain variable region that is at least 80%, at least 85%, at least 90%, or at least 95% sequence identity thereto. In certain embodiments, each of the heavy chain variable regions comprise HCDRs that do not comprise amino acid variations. In certain embodiments, each of the heavy chain variable regions do not comprise amino acid variations.

In one embodiment, the present disclosure provides a bispecific binding moiety comprising:
a FAP binding domain as described herein comprising a heavy chain variable region having an amino acid sequence as set forth in SEQ ID NO: 19, or a heavy chain variable region that is at least 80%, at least 85%, at least 90%, or at least 95% sequence identity thereto; and
a TGF-βRII binding domain as described herein comprising a heavy chain variable region having an amino acid sequence as set forth in SEQ ID NO: 27, or a heavy chain variable region that is at least 80%, at least 85%, at least 90%, or at least 95% sequence identity thereto. In certain embodiments, each of the heavy chain variable regions comprise HCDRs that do not comprise amino acid variations. In certain embodiments, each of the heavy chain variable regions do not comprise amino acid variations.

In one embodiment, the present disclosure provides a bispecific binding moiety comprising:
a FAP binding domain as described herein comprising a heavy chain variable region having an amino acid sequence as set forth in SEQ ID NO: 15, or a heavy chain variable region that is at least 80%, at least 85%, at least 90%, or at least 95% sequence identity thereto; and
a TGF-βRII binding domain as described herein comprising a heavy chain variable region having an amino acid sequence as set forth in SEQ ID NO: 35, or a heavy chain variable region that is at least 80%, at least 85%, at least 90%, or at least 95% sequence identity thereto. In certain embodiments, each of the heavy chain variable regions comprise HCDRs that do not comprise amino acid variations. In certain embodiments, each of the heavy chain variable regions do not comprise amino acid variations.

In one embodiment, the present disclosure provides a bispecific binding moiety comprising:
a FAP binding domain as described herein comprising a heavy chain variable region having an amino acid sequence as set forth in SEQ ID NO: 11, or a heavy chain variable region that is at least 80%, at least 85%, at least 90%, or at least 95% sequence identity thereto; and
a TGF-βRII binding domain as described herein comprising a heavy chain variable region having an amino acid sequence as set forth in SEQ ID NO: 23, or a heavy chain variable region that is at least 80%, at least 85%, at least 90%, or at least 95% sequence identity thereto,
wherein the FAP binding domain and TGF-βRII binding domain comprise a light chain variable region having an amino acid sequence as set forth in SEQ ID NO: 52, or a light chain variable region that is at least 80%, at least 85%, at least 90%, or at least 95% sequence identity thereto. In certain embodiments, each of the heavy chain variable regions and light chain variable regions comprise HCDRs and LCDRs, respectively, that do not comprise amino acid variations. In certain embodiments, the each of the heavy chain variable regions and light chain variable regions do not comprise amino acid variations.

In one embodiment, the present disclosure provides a bispecific binding moiety comprising:
a FAP binding domain as described herein comprising a heavy chain variable region having an amino acid sequence as set forth in SEQ ID NO: 15, or a heavy chain variable region that is at least 80%, at least 85%, at least 90%, or at least 95% sequence identity thereto; and
a TGF-βRII binding domain as described herein comprising a heavy chain variable region having an amino acid sequence as set forth in SEQ ID NO: 31, or a heavy chain variable region that is at least 80%, at least 85%, at least 90%, or at least 95% sequence identity thereto,
wherein the FAP binding domain and TGF-βRII binding domain comprise a light chain variable region having an amino acid sequence as set forth in SEQ ID NO: 52, or a light chain variable region that is at least 80%, at least 85%, at least 90%, or at least 95% sequence identity thereto. In certain embodiments, each of the heavy chain variable regions and light chain variable regions comprise HCDRs and LCDRs, respectively, that do not comprise amino acid variations. In certain embodiments, the each of the heavy chain variable regions and light chain variable regions do not comprise amino acid variations.

In one embodiment, the present disclosure provides a bispecific binding moiety comprising:
a FAP binding domain as described herein comprising a heavy chain variable region having an amino acid sequence as set forth in SEQ ID NO: 19, or a heavy chain variable region that is at least 80%, at least 85%, at least 90%, or at least 95% sequence identity thereto; and
a TGF-βRII binding domain as described herein comprising a heavy chain variable region having an amino acid sequence as set forth in SEQ ID NO: 27, or a heavy chain variable region that is at least 80%, at least 85%, at least 90%, or at least 95% sequence identity thereto,
wherein the FAP binding domain and TGF-βRII binding domain comprise a light chain variable region having an amino acid sequence as set forth in SEQ ID NO: 52, or a light chain variable region that is at least 80%, at least 85%, at least 90%, or at least 95% sequence identity thereto. In certain embodiments, each of the heavy chain variable regions and light chain variable regions comprise HCDRs and LCDRs, respectively, that do not comprise amino acid variations. In certain embodiments, the each of the heavy chain variable regions and light chain variable regions do not comprise amino acid variations.

In one embodiment, the present disclosure provides a bispecific binding moiety comprising:
a FAP binding domain as described herein comprising a heavy chain variable region having an amino acid sequence as set forth in SEQ ID NO: 15, or a heavy chain variable region that is at least 80%, at least 85%, at least 90%, or at least 95% sequence identity thereto; and
a TGF-βRII binding domain as described herein comprising a heavy chain variable region having an amino acid sequence as set forth in SEQ ID NO: 35, or a heavy chain variable region that is at least 80%, at least 85%, at least 90%, or at least 95% sequence identity thereto,
wherein the FAP binding domain and TGF-βRII binding domain comprise a light chain variable region having an amino acid sequence as set forth in SEQ ID NO: 52, or a light chain variable region that is at least 80%, at least 85%, at least 90%, or at least 95% sequence identity thereto. In certain embodiments, each of the heavy chain variable regions and light chain variable regions comprise HCDRs and LCDRs, respectively, that do not comprise amino acid variations. In certain embodiments, the each of the heavy chain variable regions and light chain variable regions do not comprise amino acid variations.

The present invention also provides a bispecific binding moiety comprising a FAP binding domain and a TGF-βRII binding domain, wherein the affinity of the FAP binding domain for human FAP is between 25-50 fold higher than the affinity of TGF-βRII binding domain for human TGF-βRII. In certain embodiments, the affinity is determined as the equilibrium dissociation constant (K_{D}). In certain embodiments, the K_{D} of the FAP binding domain for human FAP is between 25-50 fold lower than the K_{D} of TGF-βRII binding domain for human TGF-βRII, as determined in SPR. In certain embodiments, the K_{D} is measured using surface plasmon resonance (SPR).

SPR is an assay that measures binding affinity using a biosensor system such as Biacore®, or Solution Equilibrium Titration (SET) (see Friguet B et al. (1985) J. Immunol Methods; 77(2): 305-319, 25 and Hanel C et al. (2005) Anal Biochem; 339(1): 182-184). In certain embodiments, K_{D} is measured using SPR as described in Example 14.

In certain embodiments, the affinity of the FAP binding domain of the bispecific binding moiety as described herein, is in the range of about 0.1-0.2 nM, as measured by SPR as described in Example 14. In certain embodiments, the affinity of the TGF-βRII binding domain of the bispecific binding moiety, is in the range of about 3.8-5 nM, as measured by SPR as described in Example 14.

In certain embodiments, the binding affinity is measured with the FAP×TGF-βRII bispecific binding moiety of the present disclosure in bivalent bispecific format. The binding affinity of the bispecific binding moiety for human FAP and for human TGF-βRII thus represents a monovalent binding affinity.

In certain embodiments, the present disclosure also provides a bispecific binding moiety comprising a FAP binding domain and a TGF-βRII binding domain, wherein the FAP binding domain binds to human FAP (huFAP) and is cross-reactive with cynomolgus FAP (cyFAP). In certain embodiments, the present disclosure provides a bispecific binding moiety comprising a FAP binding domain and a TGF-βRII binding domain, wherein the TGF-βRII binding domain binds to human TGF-βRII (hu TGF-βRII) and is cross-reactive with cynomolgus TGF-βRII (cyTGF-βRII).

In certain embodiments, a bispecific binding moiety of the present disclosure comprises a Fab domain that binds FAP, a Fab domain that binds TGF-βRII and an Fc region.

In certain embodiments, a binding moiety comprising a polypeptide or binding domain of the present disclosure has Fc effector function.

In certain embodiments, the Fc region of the bispecific binding moiety has antibody-dependent cellular cytotoxicity (ADCC) activity. In certain embodiments, the Fc region of the bispecific binding moiety has antibody-dependent cell phagocytosis (ADCP) activity. In certain embodiments, the Fc region of the bispecific binding moiety has ADCC activity and ADCP activity.

In certain embodiments, a bispecific binding moiety of the present disclosure has an unmodified immune cell effector function, or a modified immune cell effector function. In certain embodiments, an unmodified immune effector function is caused by the bispecific binding moiety comprising an Fc region comprising a hinge, CH2, and CH3 region of an IgG1 isotype, according the SEQ ID NO: 40, 41, 43, respectively.

In certain embodiments, a bispecific binding moiety of the present disclosure has a modified immune cell effector function, such as for instance, an enhanced immune cell effector function or a reduced immune cell effector function. In certain embodiments, a modified immune effector function is caused by one or more variations in the hinge, CH2 and/or CH3 region of SEQ ID NO: 40, 41, 43, respectively.

In certain embodiments, the Fc region of a bispecific binding moiety of the present disclosure has enhanced or reduced immune effector function.

In certain embodiments, the Fc region of the bispecific binding moiety has enhanced immune cell effector function, in particular enhanced ADCC activity. A bispecific binding moiety comprising an Fc with enhanced immune effector function is referred to herein as “Fc-enhanced variant”. The immune cell effector function exhibited by the Fc-enhanced variant is enhanced when compared to the immune cell effector function exhibited by the unmodified bispecific binding moiety.

In certain embodiments, the Fc region of the bispecific binding moiety has enhanced ADCC activity.

In certain embodiments, the Fc region of the bispecific binding moiety has ADCP activity and enhanced ADCC activity.

In certain embodiments, the Fc region of the bispecific binding moiety has enhanced ADCP activity.

In certain embodiments, the Fc region of the bispecific binding moiety has enhanced ADCP activity and enhanced ADCC activity.

A bispecific binding moiety, such as an antibody, can be engineered to enhance the ADCC activity. Methods for doing so are well known in the art to persons of ordinary skill (for review, see Kubota T et al. Cancer Sci. 2009; 100(9): 1566-72). For instance, ADCC activity of an antibody can be improved by slightly modifying the constant region of the antibody (Junttila T T. et al. Cancer Res. 2010; 70(11):4481-9). Changes are sometimes also made to improve storage or production or to remove C-terminal lysines (Kubota T et al. Cancer Sci. 2009; 100(9): 1566-72). Another way to improve ADCC activity of an antibody is by enzymatically interfering with the glycosylation pathway resulting in a reduced fucose (von Horsten H H. et al. Glycobiology. 2010; 20(12): 1607-18). Alternatively, or additionally, multiple other strategies can be used to achieve ADCC enhancement, for instance including glycoengineering (Kyowa Hakko/Biowa, GlycArt (Roche) and Eureka Therapeutics) and mutagenesis, all of which seek to improve Fc binding to low-affinity activating FcγRIIIa, and/or to reduce binding to the low affinity inhibitory FcγRIIb. In certain embodiments, a bispecific binding moiety of the present disclosure exhibits enhanced ADCC.

The present disclosure therefore provides a bispecific binding moiety comprising a FAP binding domain and a TGF-βRII binding domain, wherein the bispecific binding moiety has enhanced immune cell effector function. In certain embodiments, the bispecific binding moiety is afucosylated.

In certain embodiments, an immune cell with effector function is an NK cell, a T cell, a B cell, a monocyte, a macrophage, a dendritic cell or a neutrophilic granulocyte.

In certain embodiments, the Fc region of the bispecific binding moiety has reduced immune cell effector function, in particular reduced ADCC and/or ADCP activity. A bispecific binding moiety comprising an Fc with reduced immune effector function is referred to herein as an “Fc-silenced variant”. The immune cell effector function exhibited by the Fc-silenced variant is reduced when compared to the immune cell effector function exhibited by the unmodified bispecific binding moiety.

In certain embodiments, a bispecific binding moiety of the present disclosure has reduced Fc-receptor interaction or reduced C1q binding. In certain embodiments, a bispecific binding moiety of the present disclosure exhibits reduced ADCC and/or ADCP. A bispecific binding moiety, such as an antibody, can be engineered to reduce the ADCC and/or ADCP activity (Liu R, et. al. Fc-Engineering for Modulated Effector Functions-Improving Antibodies for Cancer Treatment. Antibodies (Basel). 2020 Nov. 17; 9(4):64). For instance, ADCC and/or ADCP activity of an antibody can be reduced by modifying the CH2 and/or lower hinge region of an IgG antibody, such that the interaction of the antibody to a Fc-gamma receptor is reduced. Such a variant IgG1 CH2 and/or lower hinge region comprises an amino acid substitution at position 235 and/or 236 (EU numbering), such as for instance an L235G and/or G236R substitution.

The present disclosure, therefore also provides a bispecific binding moiety comprising a FAP binding domain and a TGF-βRII binding domain, wherein the bispecific binding moiety has reduced immune cell effector function. In certain embodiments, the bispecific binding moiety has L235G and/or G236R mutations in the CH2 domain of the Fc region (SEQ ID NO: 42) (EU numbering).

Several in vitro methods exist for determining the efficacy of antibodies or effector cells in eliciting ADCC. Among these are chromium-51 [Cr51] release assays, europium [Eu] release assays, and sulfur-35 [S35] release assays. Usually, a labeled target cell line expressing a certain surface exposed antigen is incubated with an antibody specific for that antigen. After washing, effector cells expressing Fc receptor CD16 are co-incubated with the antibody-labeled target cells. Target cell lysis is subsequently measured by release of intracellular label by a scintillation counter or spectrophotometry.

In certain embodiments, potency of bispecific binding moieties in eliciting ADCC is determined by methods as described in Example 10 or 11.

In certain embodiments, the potency of bispecific binding moieties in eliciting ADCP is determined by the method as described in Example 17. In short, the method described in Example 17 involves differentiation of human peripheral blood monocytes into M0/M2C macrophages, which are validated to express M1/M2 markers. ADCP assay is then performed in the presence of bispecific binding moieties of the present disclosure, using differentiated macrophages used as effector cells and A549-FAP⁺ cells or Lung CAFs as target cells. The ability of bispecific binding moieties to mediate ADCP on target cells can be measured by flow cytometry or imaging.

In certain embodiments, the present disclosure provides a bispecific binding moiety that competes with a bispecific binding moiety as described herein for binding to huFAP and huTGF-βRII.

For the purpose of the present disclosure, “compete”, “competes”, or “competing” refers to an activity of a bispecific binding moiety that displaces a bispecific binding moiety as described herein from its target antigen, in a cross-blocking assay. Therefore, in certain embodiments, a binding moiety that competes for binding with the bispecific binding moiety as described herein, binds to huFAP and huTGF-βRII and displaces the bispecific moiety as described herein, in a cross-blocking assay. In certain embodiments, a cross-blocking assay is a competitive ELISA. Methods of performing a competitive ELISA are known to a person of ordinary skill in the art.

In brief, in a competitive ELISA, antigen is coated on the wells of a microtiter plate and pre-incubated with or without the competing binding moiety. This is followed by addition of a biotin-labeled bispecific binding moiety as disclosed herein. The amount of labeled bispecific binding moiety bound to the antigen in the wells is measured using avidin-peroxidase conjugate and appropriate substrate. The amount of labeled bispecific binding moiety that is bound to the antigen has an indirect correlation to the ability of the competing binding moiety to compete for binding to the same antigen, i.e., the greater the affinity of the competing binding moiety for the same antigen, the less labeled bispecific binding moiety will be bound to the antigen-coated wells. A candidate competing binding moiety is considered to compete for binding to the antigen as a bispecific binding moiety of the current disclosure, if the candidate binding moiety can block binding of the bispecific binding moiety, to each target antigen, by at least 20%, in particular by at least 20-50%, in particular, by at least 50% as compared to the control performed in parallel in the absence of the candidate competing binding moiety.

### Nucleic Acids, Vectors, and Cells

Further provided herein is a nucleic acid useful for producing a polypeptide, binding domain, or binding moiety, of the present disclosure. In certain embodiments, such nucleic acid comprises a nucleic acid sequence encoding a polypeptide as described herein.

Further provided herein are nucleic acids useful for producing a bispecific binding moiety of the present disclosure. In certain embodiments, such nucleic acids comprise a nucleic acid sequence encoding the heavy chain variable region of a FAP binding domain as described herein. In certain embodiments, such nucleic acids comprise a nucleic acid sequence encoding the heavy chain variable region of a FAP binding domain and the heavy chain variable region of a TGF-βRII binding domain, as described herein.

In certain embodiments, a nucleic acid of the present disclosure may further comprise a nucleic acid sequence encoding a CH1 region and preferably a hinge, CH2 and CH3 region. In certain embodiments, the nucleic acids of the present disclosure may further comprise at least one nucleic acid sequence encoding a light chain variable region, and preferably a CL region. In certain embodiments, the light chain variable region can be a light chain variable region as described herein. In certain embodiments, the light chain variable region is a light chain variable region of a light chain that is capable of pairing with multiple heavy chains having different epitope specificities.

Further provided herein is a vector comprising a nucleic acid of the present disclosure useful for producing a binding domain or binding moiety of the present disclosure. In certain embodiments, such vector comprises a nucleic acid sequence encoding a polypeptide as described herein.

Further provided herein are vectors comprising nucleic acids of the present disclosure useful for producing a bispecific binding moiety of the present disclosure. In certain embodiments, such vectors comprise a nucleic acid sequence encoding the heavy chain variable region of a FAP binding domain as described herein. In certain embodiments, such vectors comprise a nucleic acid sequence encoding the heavy chain variable region of a FAP binding domain and the heavy chain variable region of a TGF-βRII binding domain, as described herein.

In certain embodiments, a vector of the present disclosure may further comprise a nucleic acid sequence encoding a CH1 region and preferably a hinge, CH2 and CH3 region. In certain embodiments, the vector of the present disclosure may further comprise at least one nucleic acid sequence encoding a light chain variable region, and preferably a CL region. In certain embodiments, the light chain variable region can be a common light chain variable region as described herein. In certain embodiments, the light chain variable region is a light chain variable region of a light chain that is capable of pairing with multiple heavy chains having different specificities.

The present disclosure also provides a cell comprising a nucleic acid, for example as part of a vector, comprising a sequence that encodes a polypeptide as described herein.

The present disclosure also provides a cell comprising a nucleic acid sequence, for example as part of a vector, encoding the heavy chain variable region of a FAP binding domain as described herein and a nucleic acid sequence encoding the heavy chain variable region of a TGF-βRII binding domain as described herein.

In certain embodiments, a cell of the present disclosure may further comprise a nucleic acid sequence, for example as part of a vector, encoding a CH1 region and preferably a hinge, CH2 and CH3 region. In certain embodiments, the cell of the present disclosure may further comprise at least one nucleic acid sequence, for example as part of a vector, encoding a light chain variable region, and preferably a CL region. In certain embodiments, the light chain variable region can be a common light chain variable region as described herein.

The present disclosure also provides a cell producing a polypeptide, binding domain or binding moiety as described herein. The present disclosure also provides a cell producing a bispecific binding moiety as described herein. In certain embodiments, such cell can be a recombinant cell, which comprises a nucleic acid, for example a vector, of the present disclosure. In certain embodiments, a cell of the present disclosure comprises a nucleic acid sequence, for example a vector, comprising a sequences that encodes a polypeptide as described herein. In certain embodiments, a cell of the present disclosure comprises a nucleic acid sequence, for example a vector, encoding the heavy chain variable region of a FAP binding domain as described herein and a nucleic acid sequence encoding the heavy chain variable region of a TGF-βRII binding domain as described herein. In certain embodiments, a cell of the present disclosure further comprises a nucleic acid sequence, for example a vector, encoding a CH1 region and preferably a hinge, CH2 and CH3 region. In certain embodiments, a cell of the present disclosure further comprises at least one nucleic acid sequence, for example a vector, encoding a light chain variable region, in particular a light chain variable region as described herein, and preferably a CL region.

### Pharmaceutical Compositions and Methods of Use

In certain embodiments, the present disclosure provides a pharmaceutical composition comprising an effective amount of a polypeptide as described herein, or a FAP binding domain as described herein, or a binding moiety as described herein, and a pharmaceutically acceptable carrier.

In certain embodiments, the present disclosure provides a pharmaceutical composition comprising an effective amount of a bispecific binding moiety as described herein, and optionally a pharmaceutically acceptable carrier.

In certain embodiments, the present disclosure provides a polypeptide as described herein, or a FAP binding domain as described herein, or a binding moiety as described herein, or a pharmaceutical composition as described herein, for use in therapy.

In certain embodiments, the present disclosure provides a bispecific binding moiety as described herein, and a pharmaceutical composition as described herein, for use in therapy.

In certain embodiments, the present disclosure provides a polypeptide as described herein, or a FAP binding domain as described herein, or a binding moiety as described herein, or a pharmaceutical composition as described herein, for use in the treatment of cancer.

In certain embodiments, the present disclosure provides a bispecific binding moiety as described herein, and a pharmaceutical composition as described herein, for use in the treatment of cancer.

In certain embodiments, the present disclosure provides a method for treating a disease, comprising administering an effective amount of a polypeptide as described herein, or a FAP binding domain as described herein, or a binding moiety as described herein, or a pharmaceutical composition as described herein, to an individual in need thereof.

In certain embodiments, the present disclosure provides a method for treating a disease, comprising administering an effective amount of a bispecific binding moiety as described herein, or the pharmaceutical composition as described herein, to an individual in need thereof.

In certain embodiments, the present disclosure provides a method for treating cancer, comprising administering an effective amount of a polypeptide as described herein, or a FAP binding domain as described herein, or a binding moiety as described herein, or a pharmaceutical composition as described herein, to an individual in need thereof.

In certain embodiments, the present disclosure provides a method for treating cancer, comprising administering an effective amount of a bispecific binding moiety as described herein, or the pharmaceutical composition as described herein, to an individual in need thereof.

### Combination Therapy

The bispecific binding moiety of the present disclosure may be particularly effective when used in combination with a programmed cell death protein 1 (PD-1) inhibitor. The combination of a bispecific binding moiety as described herein with a PD-1 inhibitor provides for inhibition of the PD-1/PD-L1 axis, in addition to alleviation of immune cell inhibition mediated by TGF-βRII signaling on T cells. The present disclosure therefore also provides a combination of a bispecific binding moiety as described herein and a PD-1 inhibitor. The PD-1 inhibitor may be any PD-1 inhibitor. In certain embodiments, the PD-1 inhibitor is an anti-PD-1 binding moiety. In certain embodiments, the PD-1 inhibitor is an anti-PD-1 antibody. In certain embodiments the anti-PD-1 antibody is a full length antibody, a Fab, a modified Fab, or a scFv. The anti-PD-1 antibody can be a commercially available antibody such as for instance pembrolizumab, retifanlimab, nivolumab, cemiplimab, dostarlimab, or an analog or variant thereof. In certain embodiments, the PD-1 antibody is pembrolizumab. In certain embodiments, the PD-1 antibody is retifanlimab.

PD-1 is a cell surface receptor that belongs to the CD28 family of receptors and is expressed on T cells and pro-B cells. PD-1 is presently known to bind two ligands, PD-L1 and PD-L2. PD-1, functioning as an immune checkpoint, plays an important role in down regulating the immune system by inhibiting the activation of T-cells, which in turn reduces autoimmunity and promotes self-tolerance. The inhibitory effect of PD-1 is thought to be accomplished through a dual mechanism of promoting apoptosis (programmed cell death) in antigen specific T-cells in lymph nodes while simultaneously reducing apoptosis in regulatory T cells (suppressor T cells). PD-1 is also known under a number of different aliases such as PDCD1; Programmed Cell Death 1; Systemic Lupus Erythematosus Susceptibility 2; Protein PD-1; HPD-1; PD1; Programmed 5 Cell Death 1 Protein; CD279 Antigen; CD279; HPD-L; HSLE1; SLEB2; and PD-1. External Ids for PD-1 are HGNC: 8760; Entrez Gene: 5133; Ensembl: ENSG00000188389; OMIM: 600244; and UniProtKB: Q15116. The amino acid sequence of human PD-1 is provided as SEQ ID NO: 50. New classes of drugs that block the activity of PD-1, the PD-1 inhibitors, activate the immune system to attack tumors and are therefore used with a certain level of success to treat some types of cancer.

The present disclosure further provides a kit of parts comprising a bispecific binding moiety as described herein and a PD-1 inhibitor. The present disclosure also provides a kit of parts comprising a bispecific binding moiety as described herein and instructions to use the bispecific binding moiety in combination with a PD-1 inhibitor.

The bispecific binding moiety and the PD-1 inhibitor may be formulated and/or administered together or separately, simultaneously or consecutively.

The present disclosure further provides a combination of a bispecific binding moiety as described herein and a PD-1 inhibitor for alleviating inhibitory signals in T cells. The present disclosure further provides a combination of a bispecific binding moiety as described herein and a PD-1 inhibitor for use in therapy. The present disclosure further provides a combination of a bispecific binding moiety as described herein and a PD-1 inhibitor for use in the treatment of a subject in need thereof, in particular for use in the treatment of cancer. The bispecific binding moiety as described herein and the PD-1 inhibitor may be administered simultaneously, or sequentially with the PD-1 inhibitor preceding or following the administration of the bispecific binding moiety.

The present disclosure further provides a bispecific binding moiety as described herein and a PD-1 inhibitor for use in therapy. The present disclosure further provides a bispecific binding moiety as described herein and a PD-1 inhibitor for use in the treatment of cancer.

The present disclosure further provides a bispecific binding moiety as described herein for use in therapy, wherein the therapy further comprises administering a PD-1 inhibitor. The present disclosure further provides a bispecific binding moiety as described herein for use in the treatment of cancer, wherein the treatment further comprises administering a PD-1 inhibitor. In certain embodiments, the present disclosure provides a method for treating a disease, in particular cancer, comprising administering an effective amount of a combination of a bispecific binding moiety as described herein and a PD-1 inhibitor to an individual in need thereof.

In certain embodiments, the present disclosure provides a method for treating a disease, in particular cancer, comprising administering an effective amount of a bispecific binding moiety as described herein and a PD-1 inhibitor to an individual in need thereof.

In certain embodiments, the present disclosure provides the use of a bispecific binding moiety as disclosed herein and a PD-1 inhibitor, in the manufacture of a medicament for the treatment of a disease in a subject. In certain embodiments, the bispecific binding moiety as disclosed herein and the PD-1 inhibitor are administered in separate dosage forms. In certain embodiments, the bispecific binding moiety as disclosed herein and the PD-1 inhibitor are administered simultaneously or sequentially.

As used herein, the terms “individual”, “subject” and “patient” are used interchangeably and refer to a mammal such as a human, mouse, rat, hamster, guinea pig, rabbit, cat, dog, monkey, cow, horse, pig and the like, and in particular to a human subject having cancer.

The terms “treat,” “treating,” and “treatment,” as used herein, refer to any type of intervention or process performed on or administering an active agent or combination of active agents to a subject with the objective of curing or improving a disease or symptom thereof or which produces a positive therapeutic response. As used herein, “positive therapeutic response” refers to a treatment producing a beneficial effect, e.g. reversing, alleviating, ameliorating, inhibiting, or slowing down a symptom, complication, condition or biochemical indicia associated with a disease, as well as preventing the onset, progression, development, severity or recurrence of a symptom, complication, condition or biochemical indicia associated with a disease, such as, for example, amelioration of at least one symptom of a disease or disorder, e.g. cancer. A beneficial effect can take the form of an improvement over baseline, including an improvement over a measurement or observation made prior to initiation of therapy according to the method. For example, a beneficial effect can take the form of slowing, stabilizing, stopping or reversing the progression of a cancer in a subject at any clinical stage, as evidenced by a decrease or elimination of a clinical or diagnostic symptom of the disease, or of a marker of cancer. Effective treatment may, for example, decrease in tumor size, decrease in the presence of circulating tumor cells, reduce or prevent metastases of a tumor, slow or arrest tumor growth and/or prevent or delay tumor recurrence or relapse.

The term “therapeutic amount” or “effective amount” refers to an amount of an agent or combination of agents that treats a disease, such as cancer. In some embodiments, a therapeutic amount is an amount sufficient to delay tumor development. In some embodiments, a therapeutic amount is an amount sufficient to prevent or delay tumor recurrence.

As used herein, an effective amount of the agent or composition is one that, for example, may: (i) reduce the number of cancer cells; (ii) reduce tumor size; (iii) inhibit, retard, slow to some extent and may stop cancer cell infiltration into peripheral organs; (iv) inhibit tumor metastasis; (v) inhibit tumor growth; (vi) prevent or delay occurrence and/or recurrence of tumor; and/or (vii) relieve to some extent one or more of the symptoms associated with the cancer.

An effective amount may vary according to factors such as the disease state, age, sex, and weight of the individual to be treated, and the ability of the agent or combination of agents to elicit a desired response in the individual, which can be readily evaluated by the ordinarily skilled physician or other health care worker.

An effective amount can be administered to a subject in one or more administrations.

An effective amount can also include an amount that balances any toxic or detrimental effects of the agent or combination of agents and the beneficial effects.

The term “agent” refers to a therapeutically active substance, in the present case a polypeptide, binding domain, binding moiety or bispecific binding moiety of the present disclosure, or a pharmaceutical composition of the present disclosure.

As used herein, “to comprise” and its conjugations is used in its non-limiting sense to mean that items following the word are included, but items not specifically mentioned are not excluded.

The articles “a” and “an” are used herein to refer to one or more of the grammatical object of the article. By way of example, “an element” means one or more elements.

A reference herein to a patent document or other matter is not to be taken as an admission that that document or matter was known or that the information it contains was part of the common general knowledge at the priority date of any of the claims.

All patent and literature references cited in the present specification are hereby incorporated by reference in their entirety.

Note that in the present specification, unless stated otherwise, amino acid positions assigned to CDRs and frameworks in a variable region of an antibody or antibody fragment are specified according to Kabat's numbering (see Sequences of Proteins of Immunological Interest (National Institute of Health, Bethesda, Md., 1987 and 1991)). Amino acids in the constant regions are indicated according to the EU numbering system.

Accession numbers are primarily given to provide a further method of identification of a target, the actual sequence of the protein bound may vary, for instance because of a mutation in the encoding gene such as those occurring in some cancers or the like. An antigen binding site of a binding domain, a binding moiety or a bispecific binding moieties of the disclosure can bind the antigen and a variety of variants thereof, such as those expressed by some antigen positive immune or tumor cells. HGNC stands for the HUGO Gene nomenclature committee. The number following the abbreviation is the accession number with which information on the gene and protein encoded by the gene can be retrieved from the HGNC database. Entrez Gene provides the accession number or gene ID with which information on the gene or protein encoded by the gene can be retrieved from the NCBI (National Center for Biotechnology Information) database. Ensembl provides the accession number with which information on the gene or protein encoded by the gene can be obtained from the Ensembl database. Ensembl is a joint project between EMBL-EBI and the Wellcome Trust Sanger Institute to develop a software system which produces and maintains automatic annotation on selected eukaryotic genomes.

When herein reference is made to a gene or a protein, the reference is preferably to the human form of the gene or protein. When herein reference is made to a gene or protein reference is made both to the natural gene or protein and to variant forms of the gene or protein as can be detected in tumors, cancers and the like, preferably as can be detected in human tumors, cancers and the like.

### BRIEF DESCRIPTION OF THE DRAWINGS

The following naming convention is used herein as follows. In the Figures, certain bivalent monospecific antibodies are indicated in the format SEQ ID NO: A/B, where SEQ ID NO: A refers to the heavy chain of both binding domains and SEQ ID NO: B refers to the light chain of both binding domains. Bivalent bispecific antibodies are indicated in the format SEQ ID NO: A x B, where SEQ ID NO: A refers to the heavy chain variable region of one of the binding domains and SEQ ID NO: B refers to the heavy chain variable of the other binding domain. Both domains comprise the same light chain, as described herein.

FIG. 1 shows FAP and TGF-βRII expression levels on primary CAF cell lines. CAF cell lines include: Primary Human Bladder CAFs (BLDA); Primary Human Breast CAFs (Breast); Primary Colon Cancer CAFs (COAD1); Primary Human Head and Neck CAFs (HNSC); Primary Lung adenocarcinoma CAFs (LUAD); Primary Human Lung Squamous Cell Cancer CAFs (LUSC); Primary Melanoma CAFs (MEL); Primary Pancreatic Stellate CAFs (PAAD).

A) FAP expression levels expressed in mean fluorescence intensity (MFI).

B) TGF-βRII expression levels expressed in mean fluorescence intensity (MFI).

FIG. 2 shows pSMAD2 and IL-11 inhibition of test and control antibodies in primary CAFs.

A) pSMAD2 inhibition in primary colon CAFs, expressed in % inhibition measured at different antibody concentrations. Control antibody is a bivalent monospecific TGF-βRII antibody comprising a heavy chain having an amino acid sequence as set forth in SEQ ID NO: 1 and a light chain having an amino acid sequence as set forth in SEQ ID NO: 2. Test antibodies include a bivalent bispecific antibody comprising a FAP binding domain comprising a heavy chain variable region having an amino acid sequence as set forth in SEQ ID NO: 11 and a TGF-βRII binding domain comprising a heavy chain variable region having an amino acid sequence as set forth in SEQ ID NO: 23; a bivalent bispecific antibody comprising a FAP binding domain comprising a heavy chain variable region having an amino acid sequence as set forth in SEQ ID NO: 15 and a TGF-βRII binding domain comprising a heavy chain variable region having an amino acid sequence as set forth in SEQ ID NO: 31; and a bivalent bispecific antibody comprising a FAP binding domain comprising a heavy chain variable region having an amino acid sequence as set forth in SEQ ID NO: 19 and a TGF-βRII binding domain comprising a heavy chain variable region having an amino acid sequence as set forth in SEQ ID NO: 27.

B) IL-11 inhibition in primary colon CAFs, expressed in % inhibition measured at different antibody concentrations. Control antibody is a bivalent monospecific TGF-βRII antibody comprising a heavy chain having an amino acid sequence as set forth in SEQ ID NO: 1 and a light chain having an amino acid sequence as set forth in SEQ ID NO: 2. Test antibodies include a bivalent bispecific antibody comprising a FAP binding domain comprising a heavy chain variable region having an amino acid sequence as set forth in SEQ ID NO: 11 and a TGF-βRII binding domain comprising a heavy chain variable region having an amino acid sequence as set forth in SEQ ID NO: 23; a bivalent bispecific antibody comprising a FAP binding domain comprising a heavy chain variable region having an amino acid sequence as set forth in SEQ ID NO: 15 and a TGF-βRII binding domain comprising a heavy chain variable region having an amino acid sequence as set forth in SEQ ID NO: 31; and a bivalent bispecific antibody comprising a FAP binding domain comprising a heavy chain variable region having an amino acid sequence as set forth in SEQ ID NO: 19 and a TGF-βRII binding domain comprising a heavy chain variable region having an amino acid sequence as set forth in SEQ ID NO: 27.

C) pSMAD2 inhibition in primary lung adenocarcinoma (LUAD) CAFs, expressed in % inhibition measured at different antibody concentrations. Control antibodies are a bivalent monospecific RSV-G antibody comprising a heavy chain having an amino acid sequence as set forth in SEQ ID NO: 3 and a light chain having an amino acid sequence as set forth in SEQ ID NO: 4, and a bivalent monospecific TGF-βRII antibody comprising a heavy chain having an amino acid sequence as set forth in SEQ ID NO: 1 and a light chain having an amino acid sequence as set forth in SEQ ID NO: 2. Test antibodies include a bivalent bispecific antibody comprising a FAP binding domain comprising a heavy chain variable region having an amino acid sequence as set forth in SEQ ID NO: 11 and a TGF-βRII binding domain comprising a heavy chain variable region having an amino acid sequence as set forth in SEQ ID NO: 23; and a bivalent bispecific antibody comprising a FAP binding domain comprising a heavy chain variable region having an amino acid sequence as set forth in SEQ ID NO: 15 and a TGF-βRII binding domain comprising a heavy chain variable region having an amino acid sequence as set forth in SEQ ID NO: 31.

D) IL-11 inhibition, in primary lung adenocarcinoma (LUAD) CAFs, expressed in % inhibition measured at different antibody concentrations. Control antibodies are a bivalent monospecific RSV-G antibody comprising a heavy chain having an amino acid sequence as set forth in SEQ ID NO: 3 and a light chain having an amino acid sequence as set forth in SEQ ID NO: 4, and a bivalent monospecific TGF-βRII antibody comprising a heavy chain having an amino acid sequence as set forth in SEQ ID NO: 1 and a light chain having an amino acid sequence as set forth in SEQ ID NO: 2. Test antibodies include a bivalent bispecific antibody comprising a FAP binding domain comprising a heavy chain variable region having an amino acid sequence as set forth in SEQ ID NO: 11 and a TGF-βRII binding domain comprising a heavy chain variable region having an amino acid sequence as set forth in SEQ ID NO: 23; and a bivalent bispecific antibody comprising a FAP binding domain comprising a heavy chain variable region having an amino acid sequence as set forth in SEQ ID NO: 15 and a TGF-βRII binding domain comprising a heavy chain variable region having an amino acid sequence as set forth in SEQ ID NO: 31.

E) IL-11 inhibition in primary colon CAFs, expressed as the concentration of IL-11 in pg/ml measured at different antibody concentrations. Control antibodies are an Fc-enhanced bivalent monospecific RSV-G antibody comprising a heavy chain having an amino acid sequence as set forth in SEQ ID NO: 3 and a light chain having an amino acid sequence as set forth in SEQ ID NO: 4; an Fc-silenced bivalent monospecific RSV-G antibody comprising a heavy chain having an amino acid sequence as set forth in SEQ ID NO: 5 and a light chain having an amino acid sequence as set forth in SEQ ID NO: 4; a bivalent monospecific TGF-βRII antibody comprising a heavy chain having an amino acid sequence as set forth in SEQ ID NO: 1 and a light chain having an amino acid sequence as set forth in SEQ ID NO: 2; and an Fc-enhanced bivalent monospecific FAP antibody comprising a heavy chain having an amino acid sequence as set forth in SEQ ID NO: 7 and a light chain having an amino acid sequence as set forth in SEQ ID NO: 8. Test antibodies include an Fc-silenced bivalent bispecific antibody comprising a FAP binding domain comprising a heavy chain variable region having an amino acid sequence as set forth in SEQ ID NO: 15 and a TGF-βRII binding domain comprising a heavy chain variable region having an amino acid sequence as set forth in SEQ ID NO: 31; an Fc-enhanced bivalent bispecific antibody comprising a FAP binding domain comprising a heavy chain variable region having an amino acid sequence as set forth in SEQ ID NO: 15 and a TGF-βRII binding domain comprising a heavy chain variable region having an amino acid sequence as set forth in SEQ ID NO: 31; an Fc-silenced bivalent bispecific antibody comprising a FAP binding domain comprising a heavy chain variable region having an amino acid sequence as set forth in SEQ ID NO: 15 and a TGF-βRII binding domain comprising a heavy chain variable region having an amino acid sequence as set forth in SEQ ID NO: 35; and an Fc-enhanced bivalent bispecific antibody comprising a FAP binding domain comprising a heavy chain variable region having an amino acid sequence as set forth in SEQ ID NO: 15 and a TGF-βRII binding domain comprising a heavy chain variable region having an amino acid sequence as set forth in SEQ ID NO: 35.

FIG. 3 shows pSMAD2 inhibition in A549 parental and A549-FAP⁺ cells, expressed in % inhibition measured at different antibody concentrations.

A) pSMAD2 inhibition in A549-FAP⁺ cells induced by a bivalent bispecific antibody comprising a FAP binding domain comprising a heavy chain variable region having an amino acid sequence as set forth in SEQ ID NO: 11 and a TGF-βRII binding domain comprising a heavy chain variable region having an amino acid sequence as set forth in SEQ ID NO: 23.

B) pSMAD2 inhibition in A549 parental cells induced by a bivalent bispecific antibody comprising a FAP binding domain comprising a heavy chain variable region having an amino acid sequence as set forth in SEQ ID NO: 11 and a TGF-βRII binding domain comprising a heavy chain variable region having an amino acid sequence as set forth in SEQ ID NO: 23.

C) pSMAD2 inhibition in A549-FAP⁺ cells induced by a bivalent bispecific antibody comprising a FAP binding domain comprising a heavy chain variable region having an amino acid sequence as set forth in SEQ ID NO: 15 and a TGF-βRII binding domain comprising a heavy chain variable region having an amino acid sequence as set forth in SEQ ID NO: 31.

D) pSMAD2 inhibition in A549 parental cells induced by a bivalent bispecific antibody comprising a FAP binding domain comprising a heavy chain variable region having an amino acid sequence as set forth in SEQ ID NO: 15 and a TGF-βRII binding domain comprising a heavy chain variable region having an amino acid sequence as set forth in SEQ ID NO: 31.

E) pSMAD2 inhibition in A549-FAP⁺ cells induced by a bivalent bispecific antibody comprising a FAP binding domain comprising a heavy chain variable region having an amino acid sequence as set forth in SEQ ID NO: 19 and a TGF-βRII binding domain comprising a heavy chain variable region having an amino acid sequence as set forth in SEQ ID NO: 27.

F) pSMAD2 inhibition in A549 parental cells induced by a bivalent bispecific antibody comprising a FAP binding domain comprising a heavy chain variable region having an amino acid sequence as set forth in SEQ ID NO: 19 and a TGF-βRII binding domain comprising a heavy chain variable region having an amino acid sequence as set forth in SEQ ID NO: 27.

FIG. 4 shows the ADCC activity of antibodies in A549 parental cells vs A549-FAP⁺ cells.

A) ADCC activity of antibodies in A549 parental cells, expressed in RLU, measured at different antibody concentrations. Control antibodies are a bivalent monospecific RSV-G antibody comprising a heavy chain having an amino acid sequence as set forth in SEQ ID NO: 3 and a light chain having an amino acid sequence as set forth in SEQ ID NO: 4; a bivalent monospecific TGF-βRII antibody comprising a heavy chain having an amino acid sequence as set forth in SEQ ID NO: 1 and a light chain having an amino acid sequence as set forth in SEQ ID NO: 2; and cetuximab. Test antibodies include a bivalent bispecific antibody comprising a FAP binding domain comprising a heavy chain variable region having an amino acid sequence as set forth in SEQ ID NO: 11 and a TGF-βRII binding domain comprising a heavy chain variable region having an amino acid sequence as set forth in SEQ ID NO: 23; a bivalent bispecific antibody comprising a FAP binding domain comprising a heavy chain variable region having an amino acid sequence as set forth in SEQ ID NO: 15 and a TGF-βRII binding domain comprising a heavy chain variable region having an amino acid sequence as set forth in SEQ ID NO: 31; and a bivalent bispecific antibody comprising a FAP binding domain comprising a heavy chain variable region having an amino acid sequence as set forth in SEQ ID NO: 19 and a TGF-βRII binding domain comprising a heavy chain variable region having an amino acid sequence as set forth in SEQ ID NO: 27.

B) ADCC activity of antibodies in A549-FAP⁺ cells, expressed in RLU, measured at different antibody concentrations. Control antibodies are a bivalent monospecific RSV-G antibody comprising a heavy chain having an amino acid sequence as set forth in SEQ ID NO: 3 and a light chain having an amino acid sequence as set forth in SEQ ID NO: 4; a bivalent monospecific TGF-βRII antibody comprising a heavy chain having an amino acid sequence as set forth in SEQ ID NO: 1 and a light chain having an amino acid sequence as set forth in SEQ ID NO: 2; and cetuximab. Test antibodies include a bivalent bispecific antibody comprising a FAP binding domain comprising a heavy chain variable region having an amino acid sequence as set forth in SEQ ID NO: 11 and a TGF-βRII binding domain comprising a heavy chain variable region having an amino acid sequence as set forth in SEQ ID NO: 23; a bivalent bispecific antibody comprising a FAP binding domain comprising a heavy chain variable region having an amino acid sequence as set forth in SEQ ID NO: 15 and a TGF-βRII binding domain comprising a heavy chain variable region having an amino acid sequence as set forth in SEQ ID NO: 31; and a bivalent bispecific antibody comprising a FAP binding domain comprising a heavy chain variable region having an amino acid sequence as set forth in SEQ ID NO: 19 and a TGF-βRII binding domain comprising a heavy chain variable region having an amino acid sequence as set forth in SEQ ID NO: 27.

FIG. 5 shows the ADCC activity of control and test antibodies with or without Fc modifications.

A) ADCC activity of control antibodies on Primary Melanoma CAFs (MEL), expressed as rounded CAFs per well measured at different antibody concentrations. Control antibodies include: a bivalent monospecific RSV-G antibody comprising a heavy chain having an amino acid sequence as set forth in SEQ ID NO: 3 and a light chain having an amino acid sequence as set forth in SEQ ID NO: 4; and cetuximab.

B) ADCC activity of test antibodies on Primary Melanoma CAFs (MEL), expressed as rounded CAFs per well measured at different antibody concentrations. Test antibodies include: an Fc-unmodified bivalent bispecific antibody comprising a FAP binding domain comprising a heavy chain variable region having an amino acid sequence as set forth in SEQ ID NO: 11 and a TGF-βRII binding domain comprising a heavy chain variable region having an amino acid sequence as set forth in SEQ ID NO: 23; an Fc-silenced bivalent bispecific antibody comprising a FAP binding domain comprising a heavy chain variable region having an amino acid sequence as set forth in SEQ ID NO: 11 and a TGF-βRII binding domain comprising a heavy chain variable region having an amino acid sequence as set forth in SEQ ID NO: 23; and an Fc-enhanced bivalent bispecific antibody comprising a FAP binding domain comprising a heavy chain variable region having an amino acid sequence as set forth in SEQ ID NO: 11 and a TGF-βRII binding domain comprising a heavy chain variable region having an amino acid sequence as set forth in SEQ ID NO: 23.

C) ADCC activity of test antibodies on Primary Melanoma CAFs (MEL), expressed as rounded CAFs per well measured at different antibody concentrations. Test antibodies include: an Fc-unmodified bivalent bispecific antibody comprising a FAP binding domain comprising a heavy chain variable region having an amino acid sequence as set forth in SEQ ID NO: 15 and a TGF-βRII binding domain comprising a heavy chain variable region having an amino acid sequence as set forth in SEQ ID NO: 31; an Fc-silenced bivalent bispecific antibody comprising a FAP binding domain comprising a heavy chain variable region having an amino acid sequence as set forth in SEQ ID NO: 15 and a TGF-βRII binding domain comprising a heavy chain variable region having an amino acid sequence as set forth in SEQ ID NO: 31; and an Fc-enhanced bivalent bispecific antibody comprising a FAP binding domain comprising a heavy chain variable region having an amino acid sequence as set forth in SEQ ID NO: 15 and a TGF-βRII binding domain comprising a heavy chain variable region having an amino acid sequence as set forth in SEQ ID NO: 31.

D) ADCC activity of control antibodies on Primary Lung adenocarcinoma CAFs (LUAD), expressed as number of detectable CAFs per well measured at different antibody concentrations. Control antibodies include: a bivalent monospecific RSV-G antibody comprising a heavy chain having an amino acid sequence as set forth in SEQ ID NO: 3 and a light chain having an amino acid sequence as set forth in SEQ ID NO: 4; and cetuximab. E) ADCC activity of test antibodies on Primary Lung adenocarcinoma CAFs (LUAD), expressed as number of detectable CAFs per well measured at different antibody concentrations. Test antibodies include: an Fc-unmodified bivalent bispecific antibody comprising a FAP binding domain comprising a heavy chain variable region having an amino acid sequence as set forth in SEQ ID NO: 11 and a TGF-βRII binding domain comprising a heavy chain variable region having an amino acid sequence as set forth in SEQ ID NO: 23; an Fc-silenced bivalent bispecific antibody comprising a FAP binding domain comprising a heavy chain variable region having an amino acid sequence as set forth in SEQ ID NO: 11 and a TGF-βRII binding domain comprising a heavy chain variable region having an amino acid sequence as set forth in SEQ ID NO: 23; and an Fc-enhanced bivalent bispecific antibody comprising a FAP binding domain comprising a heavy chain variable region having an amino acid sequence as set forth in SEQ ID NO: 11 and a TGF-βRII binding domain comprising a heavy chain variable region having an amino acid sequence as set forth in SEQ ID NO: 23.

F) ADCC activity of test antibodies on Primary Lung adenocarcinoma CAFs (LUAD), expressed as number of detectable CAFs per well measured at different antibody concentrations. Test antibodies include: an Fc-unmodified bivalent bispecific antibody comprising a FAP binding domain comprising a heavy chain variable region having an amino acid sequence as set forth in SEQ ID NO: 15 and a TGF-βRII binding domain comprising a heavy chain variable region having an amino acid sequence as set forth in SEQ ID NO: 31; an Fc-silenced bivalent bispecific antibody comprising a FAP binding domain comprising a heavy chain variable region having an amino acid sequence as set forth in SEQ ID NO: 15 and a TGF-βRII binding domain comprising a heavy chain variable region having an amino acid sequence as set forth in SEQ ID NO: 31; and an Fc-enhanced bivalent bispecific antibody comprising a FAP binding domain comprising a heavy chain variable region having an amino acid sequence as set forth in SEQ ID NO: 15 and a TGF-βRII binding domain comprising a heavy chain variable region having an amino acid sequence as set forth in SEQ ID NO: 31.

G) ADCC activity of test antibodies on colon CAFs, expressed as fold induction of luciferase expression, measured at different antibody concentrations. Control antibodies include: an Fc-enhanced bivalent monospecific RSV-G antibody comprising a heavy chain having an amino acid sequence as set forth in SEQ ID NO: 3 and a light chain having an amino acid sequence as set forth in SEQ ID NO: 4; and an Fc-silenced bivalent monospecific RSV-G antibody comprising a heavy chain having an amino acid sequence as set forth in SEQ ID NO: 5 and a light chain having an amino acid sequence as set forth in SEQ ID NO: 4. Test antibodies include: an Fc-enhanced bivalent bispecific antibody comprising a FAP binding domain comprising a heavy chain variable region having an amino acid sequence as set forth in SEQ ID NO: 15 and a TGF-βRII binding domain comprising a heavy chain variable region having an amino acid sequence as set forth in SEQ ID NO: 31; an Fc-silenced bivalent bispecific antibody comprising a FAP binding domain comprising a heavy chain variable region having an amino acid sequence as set forth in SEQ ID NO: 15 and a TGF-βRII binding domain comprising a heavy chain variable region having an amino acid sequence as set forth in SEQ ID NO: 31; an Fc-enhanced bivalent bispecific antibody comprising a FAP binding domain comprising a heavy chain variable region having an amino acid sequence as set forth in SEQ ID NO: 15 and a TGF-βRII binding domain comprising a heavy chain variable region having an amino acid sequence as set forth in SEQ ID NO: 35; and an Fc-silenced bivalent bispecific antibody comprising a FAP binding domain comprising a heavy chain variable region having an amino acid sequence as set forth in SEQ ID NO: 15 and a TGF-βRII binding domain comprising a heavy chain variable region having an amino acid sequence as set forth in SEQ ID NO: 35.

FIG. 6 shows selective localization of antibodies and inhibition of TGF-βRII mediated signaling in tumor cells expressing both FAP and TGF-βRII, in an in vivo study.

A) Staining of tumor cells isolated from mice inoculated with A549 parental cells (left graph) and A549-FAP⁺ cells (right graph) with control and test antibodies, expressed as IgG+, % Live cells. Control antibodies include: a bivalent monospecific RSV-G antibody comprising a heavy chain having an amino acid sequence as set forth in SEQ ID NO: 3 and a light chain having an amino acid sequence as set forth in SEQ ID NO: 4; and a bivalent monospecific TGF-βRII antibody comprising a heavy chain having an amino acid sequence as set forth in SEQ ID NO: 1 and a light chain having an amino acid sequence as set forth in SEQ ID NO: 2. Test antibodies include: a bivalent bispecific antibody comprising a FAP binding domain comprising a heavy chain variable region having an amino acid sequence as set forth in SEQ ID NO: 19 and a TGF-βRII binding domain comprising a heavy chain variable region having an amino acid sequence as set forth in SEQ ID NO: 27; a bivalent bispecific antibody comprising a FAP binding domain comprising a heavy chain variable region having an amino acid sequence as set forth in SEQ ID NO: 11 and a TGF-βRII binding domain comprising a heavy chain variable region having an amino acid sequence as set forth in SEQ ID NO: 23; and a bivalent bispecific antibody comprising a FAP binding domain comprising a heavy chain variable region having an amino acid sequence as set forth in SEQ ID NO: 15 and a TGF-βRII binding domain comprising a heavy chain variable region having an amino acid sequence as set forth in SEQ ID NO: 31.

B) IL-11 inhibition of test control and test antibodies in A549 parental cells (left graph) and A549-FAP⁺ cells (right graph), expressed as mean fluorescence intensity (MFI). Control antibodies include: a bivalent monospecific RSV-G antibody comprising a heavy chain having an amino acid sequence as set forth in SEQ ID NO: 3 and a light chain having an amino acid sequence as set forth in SEQ ID NO: 4; and a bivalent monospecific TGF-βRII antibody comprising a heavy chain having an amino acid sequence as set forth in SEQ ID NO: 1 and a light chain having an amino acid sequence as set forth in SEQ ID NO: 2. Test antibodies include: a bivalent bispecific antibody comprising a FAP binding domain comprising a heavy chain variable region having an amino acid sequence as set forth in SEQ ID NO: 19 and a TGF-βRII binding domain comprising a heavy chain variable region having an amino acid sequence as set forth in SEQ ID NO: 27; a bivalent bispecific antibody comprising a FAP binding domain comprising a heavy chain variable region having an amino acid sequence as set forth in SEQ ID NO: 11 and a TGF-βRII binding domain comprising a heavy chain variable region having an amino acid sequence as set forth in SEQ ID NO: 23; and a bivalent bispecific antibody comprising a FAP binding domain comprising a heavy chain variable region having an amino acid sequence as set forth in SEQ ID NO: 15 and a TGF-βRII binding domain comprising a heavy chain variable region having an amino acid sequence as set forth in SEQ ID NO: 31.

C) pSMAD2 inhibition of test control and test antibodies in A549 parental cells (left graph) and A549-FAP⁺ cells (right graph), expressed as mean fluorescence intensity (MFI). Control antibodies include: a bivalent monospecific RSV-G antibody comprising a heavy chain having an amino acid sequence as set forth in SEQ ID NO: 3 and a light chain having an amino acid sequence as set forth in SEQ ID NO: 4; and a bivalent monospecific TGF-βRII antibody comprising a heavy chain having an amino acid sequence as set forth in SEQ ID NO: 1 and a light chain having an amino acid sequence as set forth in SEQ ID NO: 2. Test antibodies include: a bivalent bispecific antibody comprising a FAP binding domain comprising a heavy chain variable region having an amino acid sequence as set forth in SEQ ID NO: 19 and a TGF-βRII binding domain comprising a heavy chain variable region having an amino acid sequence as set forth in SEQ ID NO: 27; a bivalent bispecific antibody comprising a FAP binding domain comprising a heavy chain variable region having an amino acid sequence as set forth in SEQ ID NO: 11 and a TGF-βRII binding domain comprising a heavy chain variable region having an amino acid sequence as set forth in SEQ ID NO: 23; and a bivalent bispecific antibody comprising a FAP binding domain comprising a heavy chain variable region having an amino acid sequence as set forth in SEQ ID NO: 15 and a TGF-βRII binding domain comprising a heavy chain variable region having an amino acid sequence as set forth in SEQ ID NO: 31.

FIG. 7 shows in vivo tumor efficacy of test and control antibodies.

A) In vivo tumor efficacy of control antibodies: a bivalent monospecific RSV-G antibody comprising a heavy chain having an amino acid sequence as set forth in SEQ ID NO: 3 and a light chain having an amino acid sequence as set forth in SEQ ID NO: 4 having enhanced effector function dosed at 30 mg/kg (group 1); a bivalent monospecific TGF-βRII antibody comprising a heavy chain having an amino acid sequence as set forth in SEQ ID NO: 1 and a light chain having an amino acid sequence as set forth in SEQ ID NO: 2 having unmodified effector function dosed at 30 mg/kg (group 2); cetuximab dosed at 30 mg/kg (group 3); and a bivalent monospecific FAP antibody comprising a heavy chain having an amino acid sequence as set forth in SEQ ID NO: 7 and a light chain having an amino acid sequence as set forth in SEQ ID NO: 8 having enhanced Fc effector function dosed at 3 mg/kg (group 4) and 30 mg/kg (group 5).

B) In vivo tumor efficacy of control and test antibodies. Control antibodies include: a bivalent monospecific RSV-G antibody comprising a heavy chain having an amino acid sequence as set forth in SEQ ID NO: 3 and a light chain having an amino acid sequence as set forth in SEQ ID NO: 4 having enhanced effector function dosed at 30 mg/kg (group 1); a bivalent monospecific TGF-βRII antibody comprising a heavy chain having an amino acid sequence as set forth in SEQ ID NO: 1 and a light chain having an amino acid sequence as set forth in SEQ ID NO: 2 in unmodified Fc format dosed at 30 mg/kg (group 2); and cetuximab dosed at 30 mg/kg (group 3). Test antibodies include: a bivalent bispecific antibody comprising a FAP binding domain comprising a heavy chain variable region having an amino acid sequence as set forth in SEQ ID NO: 15 and a TGF-βRII binding domain comprising a heavy chain variable region having an amino acid sequence as set forth in SEQ ID NO: 31 having enhanced effector function dosed at 3 mg/kg (group 6); a bivalent bispecific antibody comprising a FAP binding domain comprising a heavy chain variable region having an amino acid sequence as set forth in SEQ ID NO: 15 and a TGF-βRII binding domain comprising a heavy chain variable region having an amino acid sequence as set forth in SEQ ID NO: 31 having enhanced effector function dosed at 30 mg/kg (group 7); and a bivalent bispecific antibody comprising a FAP binding domain comprising a heavy chain variable region having an amino acid sequence as set forth in SEQ ID NO: 15 and a TGF-βRII binding domain comprising a heavy chain variable region having an amino acid sequence as set forth in SEQ ID NO: 31 having unmodified effector function dosed at 30 mg/kg (group 8).

C) In vivo tumor efficacy of test antibodies. Inhibition of tumor growth is expressed in tumor volume in mm³ measured at different days (graph). Arrowheads indicate days on which test antibodies were administered. Test antibodies include: a bivalent bispecific antibody comprising a FAP binding domain comprising a heavy chain variable region having an amino acid sequence as set forth in SEQ ID NO: 15 and a TGF-βRII binding domain comprising a heavy chain variable region having an amino acid sequence as set forth in SEQ ID NO: 35 having enhanced effector function dosed at 30 mg/kg (group 9).

FIG. 8 shows the efficacy of control and test antibodies as single agents in a trans-activity mouse model.

A) Inhibition of tumor growth, as expressed in tumor volume in mm³ measured at different days (graph) and tumor growth inhibition (TGI) in % at day 24 (table). Control antibodies include: a bivalent monospecific RSV-G antibody comprising a heavy chain having an amino acid sequence as set forth in SEQ ID NO: 3 and a light chain having an amino acid sequence as set forth in SEQ ID NO: 4 dosed at 10 mg/kg (white circles); and a bivalent monospecific TGF-βRII antibody comprising a heavy chain having an amino acid sequence as set forth in SEQ ID NO: 1 and a light chain having an amino acid sequence as set forth in SEQ ID NO: 2 dosed at 10 mg/kg (black circles). Test antibodies include: a bivalent bispecific antibody comprising a mouse FAP binding domain and a TGF-βRII binding domain comprising a heavy chain variable region having an amino acid sequence as set forth in SEQ ID NO: 23 dosed at 1 mg/kg (black squares); and a bivalent bispecific antibody comprising a mouse FAP binding domain and a TGF-βRII binding domain comprising a heavy chain variable region having an amino acid sequence as set forth in SEQ ID NO: 23 dosed at 10 mg/kg (black triangles). * P<0.05 compared to the bivalent monospecific RSV-G antibody comprising a heavy chain having an amino acid sequence as set forth in SEQ ID NO: 3 and a light chain having an amino acid sequence as set forth in SEQ ID NO: 4.

B) Inhibition of tumor growth, as expressed in tumor volume in mm³ measured at different days (graph) and tumor growth inhibition (TGI) in % at day 24 (table). Control antibodies include: a bivalent monospecific RSV-G antibody comprising a heavy chain having an amino acid sequence as set forth in SEQ ID NO: 3 and a light chain having an amino acid sequence as set forth in SEQ ID NO: 4 dosed at 10 mg/kg (white circles); and a bivalent monospecific TGF-βRII antibody comprising a heavy chain having an amino acid sequence as set forth in SEQ ID NO: 1 and a light chain having an amino acid sequence as set forth in SEQ ID NO: 2 dosed at 10 mg/kg (black circles). Test antibodies include: a bivalent bispecific antibody comprising a mouse FAP binding domain and a TGF-βRII binding domain comprising a heavy chain variable region having an amino acid sequence as set forth in SEQ ID NO: 31 dosed at 1 mg/kg (black squares); and a bivalent bispecific antibody comprising a mouse FAP binding domain and a TGF-βRII binding domain comprising a heavy chain variable region having an amino acid sequence as set forth in SEQ ID NO: 31 dosed at 10 mg/kg (black triangles). *P<0.05 compared to the bivalent monospecific RSV-G antibody comprising a heavy chain having an amino acid sequence as set forth in SEQ ID NO: 3 and a light chain having an amino acid sequence as set forth in SEQ ID NO: 4.

FIG. 9 shows the efficacy of control and test antibodies in combination with pembrolizumab in a trans-activity mouse model.

A) Inhibition of tumor growth, as expressed in tumor volume in mm³ (graph) measured at different days and tumor growth inhibition (TGI) in % at day 24 (table). Controls include: a bivalent monospecific RSV-G antibody comprising a heavy chain having an amino acid sequence as set forth in SEQ ID NO: 3 and a light chain having an amino acid sequence as set forth in SEQ ID NO: 4; a bivalent monospecific TGF-βRII antibody comprising a heavy chain having an amino acid sequence as set forth in SEQ ID NO: 1 and a light chain having an amino acid sequence as set forth in SEQ ID NO: 2; a combination of a bivalent monospecific TGF-βRII antibody comprising a heavy chain having an amino acid sequence as set forth in SEQ ID NO: 1 and a light chain having an amino acid sequence as set forth in SEQ ID NO: 2 with pembrolizumab; and pembrolizumab. Test antibodies include: a bivalent bispecific antibody comprising a mouse FAP binding domain and a TGF-βRII binding domain comprising a heavy chain variable region having an amino acid sequence as set forth in SEQ ID NO: 23; and a combination of a bivalent bispecific antibody comprising a mouse FAP binding domain and a TGF-βRII binding domain comprising a heavy chain variable region having an amino acid sequence as set forth in SEQ ID NO: 23 with pembrolizumab. *P<0.05 compared to the bivalent monospecific RSV-G antibody comprising a heavy chain having an amino acid sequence as set forth in SEQ ID NO: 3 and a light chain having an amino acid sequence as set forth in SEQ ID NO: 4.

B) Inhibition of tumor growth, as expressed in tumor volume in mm³ measured at different days (graph) and tumor growth inhibition (TGI) in % at day 24 (table). Control antibodies include: a bivalent monospecific RSV-G antibody comprising a heavy chain having an amino acid sequence as set forth in SEQ ID NO: 3 and a light chain having an amino acid sequence as set forth in SEQ ID NO: 4; and a bivalent monospecific TGF-βRII antibody comprising a heavy chain having an amino acid sequence as set forth in SEQ ID NO: 1 and a light chain having an amino acid sequence as set forth in SEQ ID NO: 2; a combination of a bivalent monospecific TGF-βRII antibody comprising a heavy chain having an amino acid sequence as set forth in SEQ ID NO: 1 and a light chain having an amino acid sequence as set forth in SEQ ID NO: 2 with pembrolizumab; and pembrolizumab. Test antibodies include: a bivalent bispecific antibody comprising a mouse FAP binding domain and a TGF-βRII binding domain comprising a heavy chain variable region having an amino acid sequence as set forth in SEQ ID NO: 31; and a combination of bivalent bispecific antibody comprising a mouse FAP binding domain and a TGF-βRII binding domain comprising a heavy chain variable region having an amino acid sequence as set forth in SEQ ID NO: 31 with pembrolizumab. *P<0.05 compared to the bivalent monospecific RSV-G antibody comprising a heavy chain having an amino acid sequence as set forth in SEQ ID NO: 3 and a light chain having an amino acid sequence as set forth in SEQ ID NO: 4.

FIG. 10 shows TGF-βRII mediated signaling inhibition, expressed as % inhibition, by the bispecific antibodies in a trans binding assay.

A) Test antibody is a bivalent bispecific antibody comprising a FAP binding domain comprising a heavy chain variable region having an amino acid sequence as set forth in SEQ ID NO: 19 and a TGF-βRII binding domain comprising a heavy chain variable region having an amino acid sequence as set forth in SEQ ID NO: 27.

B) Control antibody is a bivalent bispecific antibody comprising a mock binding domain comprising a heavy chain variable region having an amino acid sequence as set forth in SEQ ID NO: 3 and a TGF-βRII binding domain comprising a heavy chain variable region having an amino acid sequence as set forth in SEQ ID NO: 27.

C) Test antibody is a bivalent bispecific antibody comprising a FAP binding domain comprising a heavy chain variable region having an amino acid sequence as set forth in SEQ ID NO: 15 and a TGF-βRII binding domain comprising a heavy chain variable region having an amino acid sequence as set forth in SEQ ID NO: 31.

D) Control antibody is a bivalent bispecific antibody comprising a mock binding domain comprising a heavy chain variable region having an amino acid sequence as set forth in SEQ ID NO: 3 and a TGF-βRII binding domain comprising a heavy chain variable region having an amino acid sequence as set forth in SEQ ID NO: 31.

E) Test antibody is a bivalent bispecific antibody comprising a FAP binding domain comprising a heavy chain variable region having an amino acid sequence as set forth in SEQ ID NO: 11 and a TGF-βRII binding domain comprising a heavy chain variable region having an amino acid sequence as set forth in SEQ ID NO: 23.

F) Control antibody is a bivalent bispecific antibody comprising a mock binding domain comprising a heavy chain variable region having an amino acid sequence as set forth in SEQ ID NO: 3 and a TGF-βRII binding domain comprising a heavy chain variable region having an amino acid sequence as set forth in SEQ ID NO: 23.

G) Negative control antibody is a bivalent monospecific RSV-G antibody comprising a heavy chain having an amino acid sequence as set forth in SEQ ID NO: 3 and a light chain having an amino acid sequence as set forth in SEQ ID NO: 4.

FIG. 11 shows the ADCP activity of control and test antibodies with or without Fc modifications.

A) ADCP activity of test antibodies on A549-FAP⁺ cells, expressed as % phagocytosis, measured using M2c macrophage cells and A549-FAP⁺ target cells at a ratio of 1:1 (left graph) or 3:1 (right graph). Control antibody is an IgG1 isotype antibody. Test antibodies include: an Fc-enhanced bivalent bispecific antibody comprising a FAP binding domain comprising a heavy chain variable region having an amino acid sequence as set forth in SEQ ID NO: 15 and a TGF-βRII binding domain comprising a heavy chain variable region having an amino acid sequence as set forth in SEQ ID NO: 35; an Fc-silenced bivalent bispecific antibody comprising a FAP binding domain comprising a heavy chain variable region having an amino acid sequence as set forth in SEQ ID NO: 15 and a TGF-βRII binding domain comprising a heavy chain variable region having an amino acid sequence as set forth in SEQ ID NO: 35.

B) ADCP activity of test antibodies on lung CAFs cells, expressed as green object count per image, measured using M2c macrophage cells and lung CAFs target cells at a ratio of 1:1 (left graph) or 3:1 (right graph). Control antibody is an IgG1 isotype antibody. Test antibodies include: an Fc-enhanced bivalent bispecific antibody comprising a FAP binding domain comprising a heavy chain variable region having an amino acid sequence as set forth in SEQ ID NO: 15 and a TGF-βRII binding domain comprising a heavy chain variable region having an amino acid sequence as set forth in SEQ ID NO: 35; an Fc-silenced bivalent bispecific antibody comprising a FAP binding domain comprising a heavy chain variable region having an amino acid sequence as set forth in SEQ ID NO: 15 and a TGF-βRII binding domain comprising a heavy chain variable region having an amino acid sequence as set forth in SEQ ID NO: 35.

### EXAMPLES

In the Examples, which are used to illustrate the present disclosure but are not intended to limit the disclosure in any way, the FAP binding domains comprise a heavy chain variable region as further specified herein and a CH1 region having an amino acid sequence as set forth in SEQ ID NO: 39. The FAP binding domains further comprise a light chain variable region having an amino acid sequence as set forth in SEQ ID NO: 52 and a light chain constant region having an amino acid sequence as set forth in SEQ ID NO: 51.

When screened in IgG1 format, the IgGs comprise a hinge region having an amino acid sequence as set forth in SEQ ID NO: 40, a CH2 region having an amino acid sequence as set forth in SEQ ID NO: 41, and a CH3 region having an amino acid sequence as set forth in SEQ ID NO: 43.

In the Examples, which are used to illustrate the present disclosure but are not intended to limit the disclosure in any way, each binding domain of the bispecific antibodies comprises a light chain variable region having an amino acid sequence as set forth in SEQ ID NO: 52 and a light chain constant region having an amino acid sequence as set forth in SEQ ID NO: 51.

The bispecific antibodies preferably are IgG1 antibodies comprising a CH1, hinge, CH2, and CH3. The Fc region of said IgG1 antibodies may be engineered to reduce or enhance ADCC and/or CDC activity of the antibody.

In the Examples, which are used to illustrate the present disclosure but are not intended to limit the disclosure in any way, bispecific antibodies were screened in IgG1 format, wherein the FAP binding heavy chain comprises a CH1 having an amino acid sequence as set forth in SEQ ID NO: 39, a CH2 having an amino acid sequence as set forth in SEQ ID NO: 41 or 42, and a CH3 having an amino acid sequence as set forth in SEQ ID NO: 44; and the TGF-βRII binding heavy chain comprises a CH1 having an amino acid sequence as set forth in SEQ ID NO: 39, a CH2 having an amino acid sequence as set forth in SEQ ID NO: 41 or 42, and a CH3 having an amino acid sequence as set forth in SEQ ID NO: 45.

Reference antibodies and molecules used in the Examples include:
Analog reference TGF-βRII antibody TGF1 (herein referred to as analog reference TGF1), which is a bivalent monospecific analog of TGF1 and comprises two heavy chains having an amino acid sequence as set forth in SEQ ID NO: 1 and two light chains having an amino acid sequence as set forth in SEQ ID NO: 2.
Analog reference FAP antibody sibrotuzumab (herein referred to as analog reference sibrotuzumab or sibrotuzumab analog), which is a bivalent monospecific analog of sibrotuzumab and comprises two heavy chains having an amino acid sequence as set forth in SEQ ID NO: 7 and two light chains having an amino acid sequence as set forth in SEQ ID NO: 8.
Negative control IgG1 antibody (RSV-G) (herein referred to as negative control RSV-G antibody or negative control RSV antibody), which is a bivalent monospecific antibody comprising two heavy chains having an amino acid sequence as set forth in SEQ ID NO: 3 and two light chains having an amino acid sequence as set forth in SEQ ID NO: 4, or comprising two heavy chains having an amino acid sequence as set forth in SEQ ID NO: 5 and two light chains having an amino acid sequence as set forth in SEQ ID NO: 4.
Positive control antibody cetuximab which is an anti-EGFR monoclonal antibody obtained from Refdrug, Inc (NDC #66733-958-23).
Pembrolizumab, which is a bivalent anti-PD-1 antibody, obtained from Refdrug, Inc (NDC #0006-3026-02).
An analog of reference antibody ESC11 (herein referred to as ESC11 analog), which is a bivalent monospecific analog of ESC11 and comprises two heavy chains having an amino acid sequence as set forth in SEQ ID NO: 74 and two light chains having an amino acid sequence as set forth in SEQ ID NO: 75.
A positive control IgG1 antibody against human and mouse FAP (herein referred to as positive control hu/moFAP antibody), which is a bivalent monospecific antibody comprising two heavy chains having an amino acid sequence as set forth in SEQ ID NO: 76 and two light chains having an amino acid sequence as set forth in SEQ ID NO: 77.
A negative control IgG1 antibody against tetanus toxoid (TT) (herein referred to as negative control TT antibody), which is a bivalent monospecific antibody comprising two heavy chains having an amino acid sequence as set forth in SEQ ID NO: 78 and two light chains having an amino acid sequence as set forth in SEQ ID NO: 4.
Ab0625 (R&D Systems, FAB1180P).
Small molecule inhibitor Talabostat (Gentaur, GEN2327500).

### Example 1—Generation of FAP Binding Domains and Antibodies Comprising Such Binding Domains

Binding domains, antibodies and heavy chain variable regions with binding specificity to human FAP were obtained by immunizing transgenic mice comprising a common IGKV1-39 light chain (MeMo® mice) with human FAP antigenic moieties, including the use of different forms of DNA, protein, and cell-based antigen delivery.

Phage display libraries were constructed and human FAP binders were selected by performing recombinant protein panning and cell selections. Binders were reformatted into bivalent monospecific IgG format for subsequent screening and characterization in binding and functional assays.

The binding domain sequences herein, once characterized and sequenced through the techniques provided herein, can be subsequently obtained by any method known in the art.

### Example 2—Characterization of Anti-Human FAP Bivalent Monospecific IgG's

A large and diverse panel of anti-human FAP bivalent monospecific IgG's was screened for cross-reactivity to cynomolgus FAP (cyFAP), binding to mouse FAP (moFAP), and binding to human CD26 (huCD26), in FACS assays; and for binding to WI-38 cells endogenously expressing human FAP (huFAP). The IgG's were also screened for their ability to inhibit FAP catalytic activity. Binning experiments were performed to bin the FAP binding domains in groups binding differently to human FAP.

### FACS

FACS analysis was performed to investigate the specificity, species cross-reactivity, and CD26 cross-reactivity of the anti-human FAP panel.

For this assay, 293FF-huFAP cells (stably expressing huFAP) and 2833FF-cyFAP cells (stably expressing cyFAP) were used. Moreover, 293FF cells that were transiently transfected with the following constructs were used: moFAP, and huCD26. Mock transfected 293FF cells were used to analyze background binding of the antibodies.

The following control antibodies were included: sibrotuzumab analog (as positive control for huFAP and cyFAP), positive control hu/moFAP antibody (as positive control for huFAP and moFAP), negative control TT antibody (as negative control), and Ab0625 (as positive control for huCD26).

The binding of all test and control antibodies to the cells was tested at a single concentration of 2.5 ug/ml. Goat-anti-human IgG F(ab)2-PE (Invitrogen, H10104) was used as secondary antibody (1:100 in FACS buffer). Ab0625 was used at 10 ug/ml and its binding was detected using Ab0250 (Becton Dickinson, 550767) (1:100 in FACS buffer). The FACS buffer containing PBS (Gibco, cat. #10010-015), 0.5% BSA (Thermo Scientific, cat. #37525), and 2 mM EDTA (Invitrogen, cat. #15575-020) was kept ice cold throughout the assay.

The target cells were harvested, counted, and centrifuged for 5 min at 300 g at 4ºC. Cells were then resuspended in FACS buffer at a concentration of 1×10⁶ cells/ml and transferred (200,000 cells/well) to a U-bottom 96-well FACS plate (BD, cat. #353910). Cells were then centrifuged for 3 min at 300 g at 4° C. and supernatant was discarded. 50 μl of primary antibody solutions were added to the cells, mixed, and incubated for 30 min at 2-8° C. in the dark. Cells were washed by adding 150 μl FACS buffer and centrifuged for 3 min at 300 g at 4° C. Supernatant was discarded, and cells were washed again by adding 200 μl FACS buffer to the plate. Cells were then centrifuged for 3 min at 300 g at 4° C. and afterwards the supernatant was discarded. 50 μl of secondary antibody solutions were added to the cells, mixed, and incubated for 30 min at 2-8° C. in the dark. Cells were then washed by adding 150 μl FACS buffer to the plate and centrifuged for 3 min at 300 g at 4° ° C. Supernatant was discarded and cells were washed again by adding 200 μl FACS buffer to the plate and centrifuged for 3 min at 300 g at 4° C. Supernatant was discarded and cells were fixated by adding 100 μl/well ice-cold Fixation buffer (2% formaldehyde solution in PBS, Sigma-Aldrich, cat. #47608-250ML-F). Cells were incubated for 15 min on ice in the dark. Afterwards, 100 μl ice-cold PBS was added to the cells and cells were centrifuged for 3 at 300 g at 4° C. Supernatant was discarded and cells were resuspended in 120 μl FACS buffer. The plate was then sealed with EASYseal (Greiner, cat. #67600) and stored at 4° C. in the dark until measurement. Fluorescence intensity of the cells was then measured on the iQue VBR (Intellicyt).

All IgG's screened showed binding to 293FF cells stably transfected with huFAP, as well as to 293FF cells stably transfected with cyFAP (data not shown). The IgG's showed a large range in binding activity.

Only a few of the IgG's screened showed binding to 293FF cells stably transfected with moFAP (data not shown). An example of an IgG that binds moFAP is the one comprising a heavy chain variable region having an amino acid sequence as set forth in SEQ ID NO: 69.

None of the IgG's screened showed binding to huCD26 expressing cells (data not shown).

### Binding to WI-38 Cells Endogenously Expressing Human FAP

The human anti-FAP panel was tested on WI-38 cells (endogenously expressing huFAP) and binding was measured by FACS.

The target cells were harvested, counted, and centrifuged for 5 min at 300 g at 4° C. Cells were then resuspended in FACS buffer at a concentration of 1×10⁶ cells/ml and transferred (200,000 cells/well) to a U-bottom 96-well FACS plate (BD, cat. #353910). Cells were washed by adding 200 μl PBS and centrifuged for 3 min at 300 g at 4ºC. Cells were then centrifuged for 3 min at 300 g at 4° C. and supernatant was discarded. Antibodies were diluted in an 8 steps, semi-log dilution series ranging from 10 ug/ml to 3.16 ng/ml in FACS buffer (50 μ/well), mixed, and incubated for 30 min at 2-8° C. in the dark. A titration of the sibrotuzumab analog was included as positive control and used to normalize data. The negative control TT antibody was included as negative control. Cells were washed by adding 150 μl FACS buffer and centrifuged for 3 min at 300 g at 4° C. Supernatant was discarded, and cells were washed again by adding 200 μl FACS buffer to the plate. Cells were then centrifuged for 3 min at 300 g at 4° C. and afterwards the supernatant was discarded. Goat-anti-human IgG F(ab)2-PE (Invitrogen, H10104) was used as secondary antibody (1:100 in FACS buffer) and 50 μl of secondary antibody solutions were added to the cells, mixed, and incubated for 30 min at 2-8° C. in the dark. Cells were then washed by adding 150 μl FACS buffer to the plate and centrifuged for 3 min at 300 g at 4° C. Supernatant was discarded and cells were washed again by adding 200 μl FACS buffer to the plate and centrifuged for 3 min at 300 g at 4° C. Supernatant was discarded and cells were fixated by adding 100 μl/well ice-cold Fixation buffer (2% formaldehyde solution in PBS, Sigma-Aldrich, cat. #47608-250ML-F). Cells were incubated for 15 min on ice in the dark. Afterwards, 100 μl ice-cold PBS was added to the cells and cells were centrifuged for 3 min at 300 g at 4° C. Supernatant was discarded and cells were resuspended in 120 μl FACS buffer. The plate was then sealed with EASYseal (Greiner, cat. #67600) and stored at 4° C. in the dark until measurement. Fluorescence intensity of the cells was then measured on the iQue VBR (Intellicyt).

All IgG's screened showed binding to WI-38 cells endogenously expressing human FAP (data not shown). Some IgG's, including a bivalent bispecific antibody comprising a FAP binding domain comprising a heavy chain variable region having an amino acid sequence as set forth in SEQ ID NO: 19, showed a similar or higher binding than the positive control.

### Characterization of Domain Specificity

FACS analysis was performed to investigate the domain specificity of anti-human FAP antibodies. For this assay, the same FACS assay as described above was used; however, instead of using 293FF cells transiently transfected with moFAP or huCD26, 293FF cells were transiently transfected with human FAP and chicken FAP chimeric constructs. The chimeric constructs used were: a huFAP Dom1-chFAP Dom2 chimeric construct, and a chFAP Dom1-huFAP Dom2 chimeric construct. The human FAP domain 1 is a beta propeller domain, whereas the human FAP domain 2 is an alpha-beta hydrolase domain.

All the antibodies from the panel bound to the FAP beta-propeller domain and none of the antibodies bound to the alpha-beta hydrolase domain (data not shown).

### Inhibition of FAP Catalytic Activity

The Flurogenic FAP Assay Kit (Bio-connect, cat. #80210) was used to test the ability of the IgG's to inhibit FAP catalytic activity. A series of 2-fold dilutions of the fluorescent AMC standard was made in DPP buffer as follows: 1.25 μM, 0.625 μM, 0.312 μM, 0.156 μM, 0.078 μM, 0.039 μM; 100 μl per well in a 96-well assay plate. Recombinant FAP protein (2 ng/μl, 25 μl per well) (Fluorogenic FAP Assay kit, Bio-connect, cat. #80210) was added in the 96-well assay plate, followed by the IgG's at a single concentration of 25 ug/ml (50 μl/well). ESC11 analog and the small molecule inhibitor Talabostat (at a concentration of 1 μM) (Gentaur, GEN2327500) were included as positive controls. A negative control TT antibody was included as negative control. The assay plate was incubated for 15 min at 22° C. The fluorogenic substrate Ala-Pro-AMC (2.5 μM, 25 μl/well) was then added. After 6 hours incubation at 22° C., the fluorescence (generated by cleaved fluoropore AMC) was measured on a fluorescence plate reader (excitation 380 nm and emission 460 nm).

The small molecule inhibitor Talabostat showed strong inhibition of FAP catalytic activity. None of the IgG's could inhibit FAP catalytic activity as potently as the small molecule inhibitor Talabostat in this assay (data not shown).

### Competition ELISA

ELISA was used to investigate the competitive binding of the human anti-FAP antibody panel with Fab fragments generated from the sibrotuzumab analog to huFAP-His protein (R&D Systems cat. #3715-SE).

The huFAP-His protein was coated (0.5 ug/ml, 50 μl/well) on two 96-wells ELISA plates (Certified Nunc-Immuno Maxisorp F96, Greiner Bio-One, cat. #655061). The plates were sealed (EASYseal, Greiner, cat. #676001) and incubated overnight at 4° ° C. The plates were washed with PBS/0.05% Tween 20 (PBS, Gibco, cat. #10010-015) (Tween 20, Merck, cat. #8.22184.0500) using the ELISA plate washer (BioTek 405 TS). Afterwards, the plates were emptied. Fab fragments of sibrotuzumab analog (25 ug/ml) were incubated on one plate for 30 min at RT, whereas the second plate was incubated with 300 μl/well PBS/2% BSA block buffer (BSA, Sigma, cat. #A3294-500 g) for 1 hour at RT. Afterwards, the plates were emptied. Antibodies from the human anti-FAP panel (0.05 ug/ml, 50 μl/well) were added, plates were covered with a EASYseal, and incubated for one hour at RT. The plates were washed three times with wash buffer using the ELISA washer and emptied. HRP-conjugated anti-human Fc detection antibody (1:2000 diluted, Ab #0074, Bethyl labs, A80-104P) was added to the wells (50 μl/well), covered with EASYseal and incubated for 60 min at RT. The plates were then washed three times with wash buffer using the ELISA washer and emptied. BD OptEIATMB Substrate Reagent Set (BD, cat. #555214) was then added to the wells (50 μl/well) and developed for maximal 10 min. Afterwards, 1 M H2SO4 (50 μl/well) was added to the wells to stop the staining reaction (color changes from blue to yellow). The plates were then measured using an ELISA plate reader (BioTek ELx808).

In the presence of the sibrotuzumab analog Fab fragments, the anti-FAP IgG's from the panel showed a diverse extent of binding (data not shown). Some IgG's fully competed with the sibrotuzumab analog Fabs for binding to huFAP protein, including an IgG of which the FAP binding domain comprises a heavy chain variable region having an amino acid sequence as set forth in SEQ ID NO: 15, and an IgG of which the FAP binding domain comprises a heavy chain variable region having an amino acid sequence as set forth in SEQ ID NO: 19; some IgG's partially competed; and some IgG's did not compete, including an IgG of which the FAP binding domain comprises a heavy chain variable region having an amino acid sequence as set forth in SEQ ID NO: 11. Interestingly, the extent of competition of the IgG's with the sibrotuzumab analog Fabs was not correlated with their affinities.

### Example 3—Binning

Binning was performed with AR2G Biosensors (Pall Forte Bio, cat. #18-5092) and 96-well black microplates. For this experiment, FAP binding domains were reformatted into IgG1 bispecific antibodies comprising a second binding domain that targets TGF-βRII, as described in Example 5.

FAPxTGF-βRII bispecific antibodies, sibrotuzumab analog, and the negative control TT antibody were diluted at 10 ug/ml (66.7 nM, 200 μl/well) in 1×PBS. The negative control TT antibody and acetate buffer pH 6 (used as mock immobilization) were used to identify non-specific interference during the binning assay. Each antibody that was immobilized in AR2G biosensors was incubated with 5.7 ug/ml (66.7 nM, 200 μl/well) of huFAP-His recombinant protein (R&D systems, cat. #3715-SE-010) and then sandwiched with each one of the antibodies that were used for immobilization at 10 g/ml (66.7 nM, 200 μl/well).

The binning assay was performed as follows: first the AR2G biosensors were dipped for 60 sec in Ultrapure water for equilibration, then the sensors were activated in activation reagent (20 nM of EDC (Pall ForteBio, cat. #18-1033) mixed with 10 nM S-NHS (Pall ForteBio, cat. #18-1067) in Ultrapure water (200 μl/well)) for 300 sec. Then anti-huFAP:huTGF-βRII bispecific antibodies were immobilized on the sensors for 1200 sec and quenched afterwards for 300 sec with 1M ethanolamine pH 8.5 (Pall Forté Bio, cat. no. 18-1071, 200 μl/well). The sensors are then dipped in Ultrapure water (200 μl/well) for 120 sec and 1×PBS (200 μl/well) for 300 sec. Then the binding of huFAP-His recombinant protein took place for 300 sec, followed by dipping the sensors in 1×PBS for 600 sec for dissociation. The sensors were then dipped in seven cycles of 5 sec in regeneration buffer (10 mM Glycine pH 2.5) followed by 5 sec in 1×PBS. Next the sensors are dipped in 1×PBS for 120 sec, followed by huFAP-His recombinant protein binding for 120 sec. Then the sensors are dipped in 1×PBS for 60 sec, followed by binding with anti-huFAP:huTGF-βRII bispecific antibodies, sibrotuzumab analog, and negative control TT antibody for 120 sec. Finally, the sensors are dipped in 7 cycles of 5 sec in regeneration buffer (10 mM Glycine pH 2.5) followed by 5 sec in 1×PBS.

Results are shown in Table 1. Sibrotuzumab analog is in bin G, sub-bin G1 (data not shown). A bivalent bispecific antibody of which the FAP binding domain comprises a heavy chain variable region having an amino acid sequence as set forth in SEQ ID NO: 19 is in the same bin (bin G1) as the sibrotuzumab analog, and a bivalent bispecific antibody of which the FAP binding domain comprises a heavy chain variable region having an amino acid sequence as set forth in SEQ ID NO: 15 or SEQ ID NO: 11 are in different bins than sibrotuzumab analog (bin F1 and B2, resp.).

From this data we can conclude that a bivalent bispecific antibody of which the FAP binding domain comprises a heavy chain variable region having an amino acid sequence as set forth in SEQ ID NO: 15 competes with sibrotuzumab for binding to human FAP but binning experiments indicate that binding of this antibody to human FAP differs from that of sibrotuzumab.

### Example 4—Selection of FAP Binding Domains for Use in Bispecific IgG Format

From the large and diverse panel of anti-human FAP bivalent monospecific IgG's that was generated, three FAP binding domains were identified to be particularly useful for the generation of bispecific antibodies: FAP binding domain comprising a heavy chain variable region having an amino acid sequence as set forth in SEQ ID NOs: 15; 19; and 11. These three FAP binding domains were combined with different TGF-βRII binding domains, as described in Example 5, and the resulting FAP×TGF-βRII bispecific antibodies were screened in an in vitro mixed culture pSMAD2 assay and an in vivo tumor targeting study.

TABLE 1
This table summarizes if IgGs comprising a FAP binding domain that comprises
a heavy chain variable region (VH) having an amino acid sequence as set forth
in SEQ ID NO: 15; 19; or 11, compete with a sibrotuzumab analog for binding to
human FAP, and the results of binning including binding affinities of
FAP × TGF-βRII bispecific antibodies comprising these FAP binding domains.

| | Competition | | | | | |
|---|---|---|---|---|---|---|
| | with | | | | | |
| | sibrotuzumab | Ka (1/Ms) | Kdis (1/s) | KD (M) | | |
| FAP VH | analog | (AVG) | (AVG) | (AVG) | Bin | Sub-bin |
| SEQ ID | No | 562333 | <1.00E−07 | 1.00E−12 | B | B2 |
| NO: 11 | competition | | | | | |
| SEQ ID | Competition | 314533 | <1.00E−07 | 1.00E−12 | F | F1 |
| NO: 15 | | | | | | |
| SEQ ID | Competition | 436033 | 6.38E−05 | 1.47E−10 | G | G1 |
| NO: 19 | | | | | | |

### Example 5: Generation of FAP×TGF-βRII Bispecific Antibodies

Binding domains, antibodies and heavy chain variable regions with binding specificity to human FAP and heavy chain variable regions with binding specificity to human TGF-βRII were obtained by immunizing transgenic mice comprising a common IGKV1-39 light chain (MeMo® mice) with human FAP or TGF-βRII antigenic moieties, including the use of different forms of DNA, protein and cell-based antigen delivery.

Heavy chain variable regions with binding specificity to TGF-βRII are also described in WO 2021/133167 as SEQ ID NO: 10-12; SEQ ID NO: 22-91 and SEQ ID NO: 93-96.

Heavy chain variable regions with binding specificity to human FAP having an amino acid sequence as set forth in SEQ ID NO: 11, 15 and 19, and heavy chain variable regions with binding specificity to human TGF-βRII having an amino acid sequence as set forth in SEQ ID NOs: 23, 27, 31, and 35, were selected for the production of bispecific antibodies. The binding domain sequences herein, once characterized and sequenced through the techniques provided herein, can be subsequently obtained by any method known in the art.

TABLE 2
Combination of FAP heavy chain variable regions and
TGF-βRII heavy chain variable regions that can
be used for the generation of bispecific antibodies.

FAP

| | | SEQ ID | SEQ ID | SEQ ID |
|---|---|---|---|---|
| | | NO: 11 | NO: 15 | NO: 19 |
| TGF-βRII | SEQ ID NO: 23 | PB1 | PB2 | PB3 |
| | SEQ ID NO: 27 | PB4 | PB5 | PB6 |
| | SEQ ID NO: 31 | PB7 | PB8 | PB9 |
| | SEQ ID NO: 35 | PB10 | PB11 | PB12 |

Bispecific IgG antibodies were generated by transient co-transfection of two plasmid vectors: one encoding an IgG heavy chain with a FAP binding VH region and the other encoding an IgG heavy chain with a TGF-βRII binding VH region. CH3 engineering technology as described in WO 2013/157954 and WO 2013/157953 was employed to ensure efficient heterodimerization and formation of bispecific antibodies. Both vectors further encode a common light chain comprising the IGKV1-39/Jk1 light chain variable region. Cell transfection, cell culture, and the harvesting and purification of antibodies was performed by methods known in the art. Further CH3 engineering technologies, for instance as described in WO 2021/235936, may be employed to ensure efficient dimerization and formation of bispecific antibodies.

Antibodies with Modified Effector Function:

Bispecific antibodies with unmodified effector function comprise a CH2 region having an amino acid sequence as set forth in SEQ ID NO: 41. Fc-silenced variants of bispecific antibodies were produced by introducing L235G and G236R mutations in the CH2 domain (SEQ ID NO: 42). Fc-enhanced variants of bispecific antibodies were produced using FUT-8 knock-out CHO cells, which generate afucosylated antibodies (Zong H, et al. Producing defucosylated antibodies with enhanced in vitro antibody-dependent cellular cytotoxicity via FUT8 knockout CHO-S cells. Eng Life Sci. 2017 Apr. 18; 17(7):801-808). An Fc-enhanced variant of analog reference FAP antibody sibrotuzumab was produced using the methods as set out in Roy G, et. al., A novel bicistronic gene design couples stable cell line selection with a fucose switch in a designer CHO host to produce native and afucosylated glycoform antibodies, MAbs, 2018 April; 10(3):416-430.

### Example 6: Primary CAF Cell Lines and Expression

Primary cancer associated fibroblast (CAF) cells were obtained and validated for use in the characterization of the bispecific antibodies.

TABLE 3
CAF cell lines used for TGF-βRII signaling inhibition
assay with selected bispecific antibodies.

| | Primary CAF Information | | | |
|---|---|---|---|---|
| Vendor | Lot ID | Cat # | Primary cancer indication | Passage |
| Bio IVT | DT01012 | HPCCAFLUN-1 | Primary Human Lung Sq Cell | 1 |
| | | | Cancer CAFs (LUSC) | |
| | DT01045 | HPCCAFBL-05 | Primary Human Bladder CAFs | 1 |
| | | | (BLDA) | |
| | DT01094 | HPCCAFBR-1 | Primary Human Breast CAFs | 2 |
| | | | (Breast) | |
| | DT01249 | HPCCAFHN-05 | Primary Human Head and Neck | 1 |
| | | | CAFs (HNSC) | |
| | 427650A1-6037; | | Primary Colon Cancer CAFs | 4 |
| | 373853A2-6020 | | (COAD1) | |
| Neuromics | 003C | CAF08 | Primary Pancreatic Stellate | 3 |
| | | | CAFs (PAAD) | |
| | oo2 | CAF09 | Primary Melanoma CAFs (MEL) | 2 |
| | oo4 | AF07-AD | Primary Lung adenocarcinoma | 4 |
| | | | CAFs (LUAD) | |
| | oo4 | CAF05 | Primary Colorectal | 4 |
| | | | adenocarcinoma CAFs (COAD2) | |
| | oo5 | CAF02 | Primary Ovarian serous (OV) | 5 |
| | oo3 | CAF03 | Primary Glioblastoma (GBM) | 3 |

CAF cells (Table 3) were obtained from BioIVT or Neuromics and shown to express both human FAP and human TGF-βRII.

huFAP was detected using primary mouse anti-FAP antibody (R&D systems, cat. no. MAB3715) and secondary FITC-conjugated goat anti-mouse antibody (Jackson I R, cat. no. 115-095-062). huTGF-βRII was detected using analog reference TGF1 antibody and secondary Alexa fluor F647 conjugated goat anti-human antibody (Jackson I R, cat no. 109-605 003). Cells were cultured in DMEM+10% FBS without Pen/Strep. On the day of assay, cells were trypsinized, washed in growth media, pelleted, and re-suspended in FACS buffer 10 (PBS/1% FBS). One million cells per well were re-suspended in 100 μl of primary antibody in PBS/1% FBS. Cells were stained for 30 minutes in the dark. Cells were then washed 2× with FACS buffer, pelleted, and stained with viability dye (Zombie Violet™ Fixable Viability Kit, BioLegend, cat. no. 423114) and secondary IgG in the dark for 25 min. Cells were washed twice with FACS buffer, pelleted, and fixed in paraformaldehyde for 15 min on ice. Cells were then washed in PBS, re-suspended in 200 μl PBS, and measured by flow cytometry. Graphs were plotted in mean fluorescence intensity (MFI).

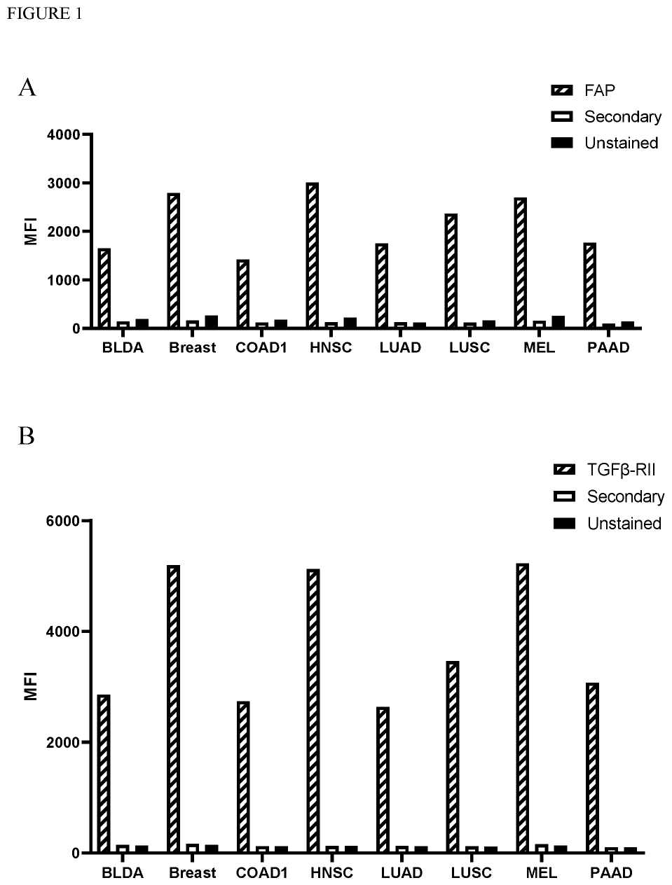

FIG. 1 shows the MFI obtained from FACS for FAP (1A) and TGF-βRII (1B) detection. All CAFs tested were found to express both FAP and TGF-βRII. The MFI of expression of both FAP and TGF-βRII was at least 2-3 fold higher compared to the background MFI obtained with only the secondary antibody staining.

### Example 7: TGF-βRII Signaling Inhibition Assay in Primary CAF Cells Expressing FAP and TGF-βRII

Bispecific antibodies were characterized in a TGF-βRII signaling inhibition assay to determine their ability to inhibit TGF-βRII mediated signaling. Expression of TGF-β induced IL-11 and pSMAD2 was used as a read-out for TGF-βRII signaling. The assay was performed using primary colon CAFs (BioIVT) which were first validated to express both FAP and TGF-βRII using FACS, as described in Example 6.

Several FAP×TGF-βRII bispecific antibodies were tested. A reference antibody included in the assay was analog reference antibody TGF1 as described herein.

IL-11 inhibition: Primary colon CAFs or other primary CAFs as indicated herein, were cultured in DMEM+10% FBS medium without antibiotics. When cells were 80-90% confluent, they were trypsinized with TrypLE (Gibco, 12604-013), pelleted at 200 g and washed 1× with DMEM+0.2% FBS. Cells were re-suspended to 1×10⁶/ml (primary colon CAFs) or 5×10⁵/ml (other CAFs) and seeded 50 ul/well (50000 cells or 25000 cells) in 96-well flat plates in DMEM+0.2% FBS. Cells were pre-incubated with bispecific or reference antibodies at room temperature for 1 hour. The antibodies were serially diluted 5-fold or 8-fold and added to wells starting at 400 ug/ml (final concentration 100 ug/ml). After 1 hr, 100 ul of 2 ng/ml (2×) of recombinant human TGF-β1 (R&D #7754-BH) was added to the cells (200 ul total volume/well) for 48 or 72 hours at 37° C. Plates were then spun down at 300 g for 3 minutes and supernatants were assayed for IL-11 expression using the Ella platform.

pSMAD2 inhibition: Primary colon CAFs or other primary CAFs as indicated herein, were cultured in DMEM+10% FBS medium without antibiotics. When cells were 80-90% confluent, they were trypsinized with TrypLE (Gibco, 12604-013), pelleted at 200 g and washed 1× with DMEM+0.2% FBS. Cells were re-suspended and seeded at 120,000 cells/well in DMEM+0.2% FBS. Cells were pre-incubated with bispecific or reference antibodies at room temperature for 1 hour. The antibodies were serially diluted 5-fold and added to wells starting at 400 ug/ml (final concentration 100 ug/ml). After 1 hr, 100 ul of 2 ng/ml (2λ) of recombinant human TGF-β1 was added to wells for 2 hours. Cells were washed in PBS and re-suspended in 50 μl of 300-fold diluted viability dye (Zombie Violet, BioLegend, cat. no. 423113) in 1% FBS/PBS for 10 min. Cells were then washed and fixed and permeabilized according to TFP protocol (Transcription Factor Phospho Buffer Set, BD Biosciences, cat. no. 563239) using 200 μl of buffer. Cells were stained with Rabbit anti-human pSMAD2 antibody (Cell Signaling, cat. no. 18338) on ice for 1 hour. Cells were then washed and stained with anti-rabbit IgG secondary antibody (JAX Immuno, cat. no. 611-605-215) for 30 minutes on ice in the dark. Following staining, cells were washed 3× in wash buffer, re-suspended in 200 μl of PBS, and cells were acquired in a Fortessa flow cytometer.

Data was plotted in GraphPad Prism and IC50 calculated using non-linear regression. Inhibition of IL-11/pSMAD2 expression in CAFs incubated with TGF-β1 and test antibodies was compared with respect to expression in the presence of TGF-β1 alone.

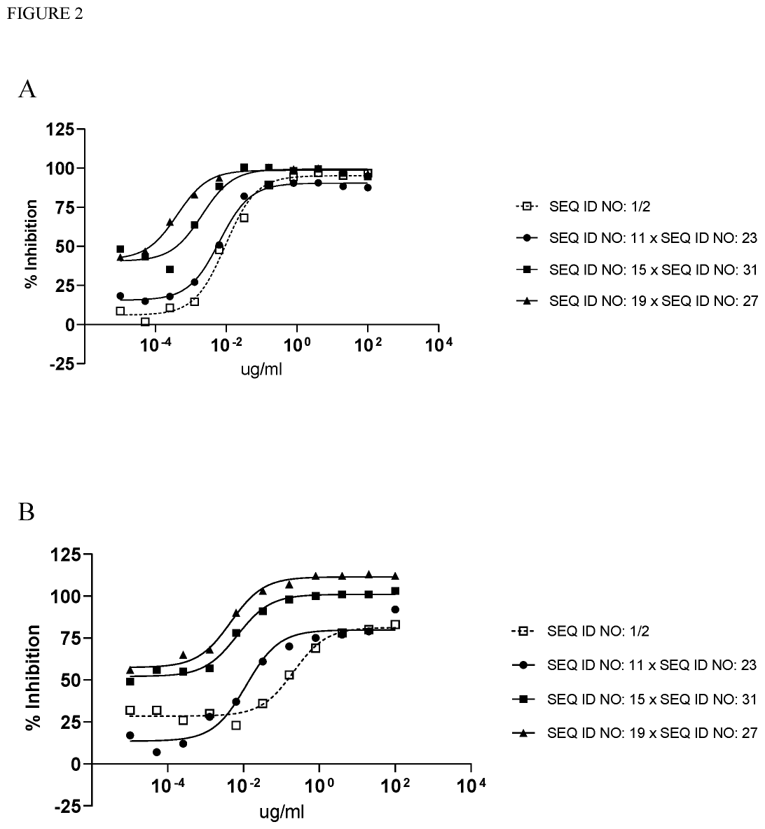

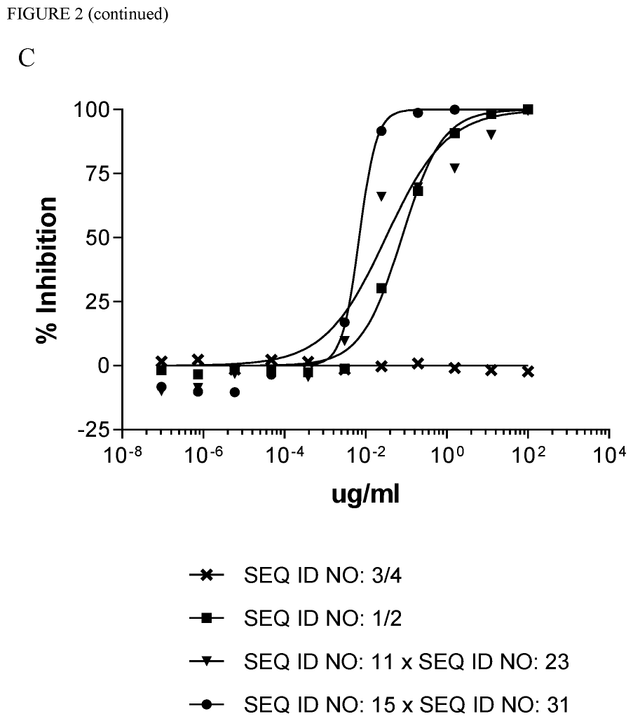

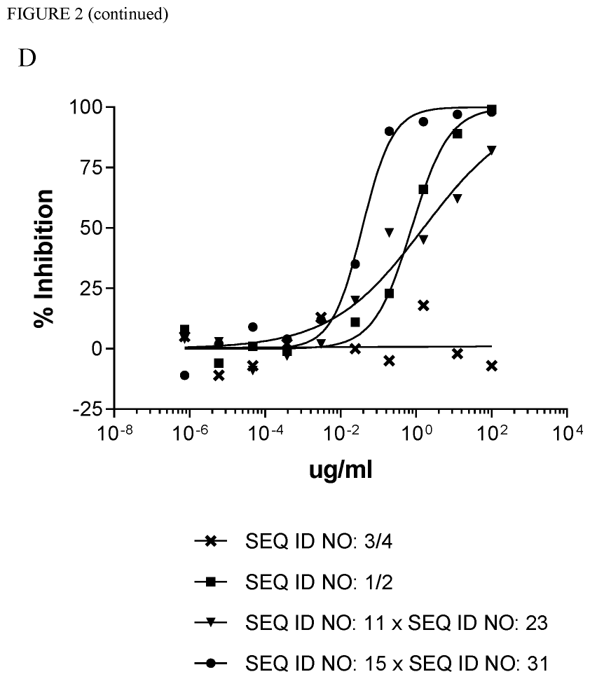

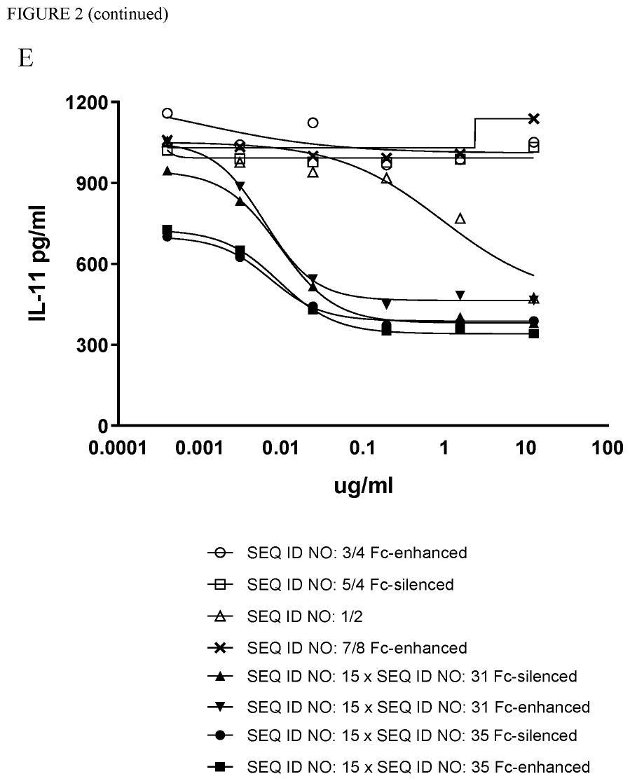

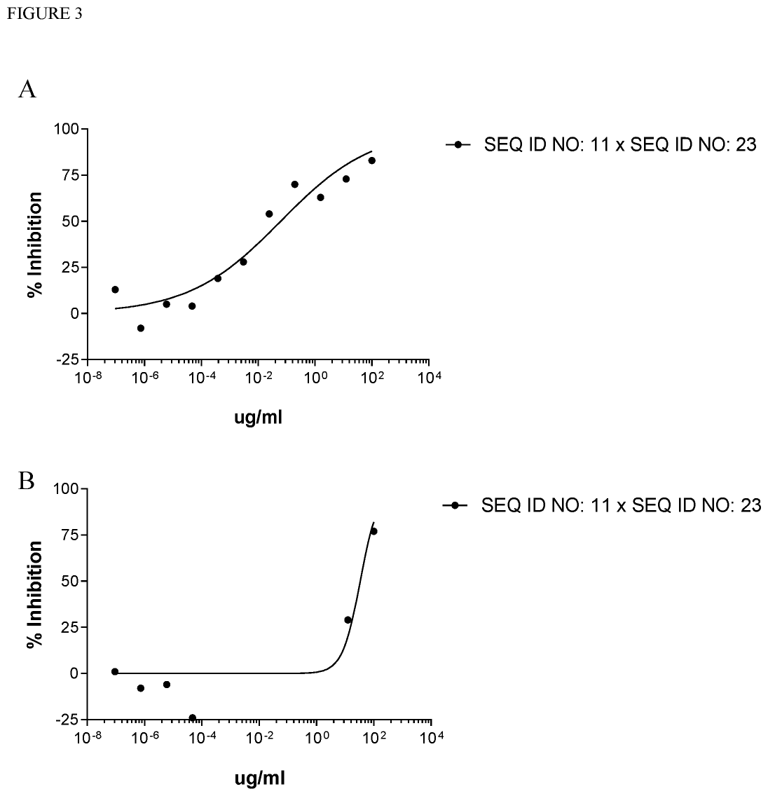

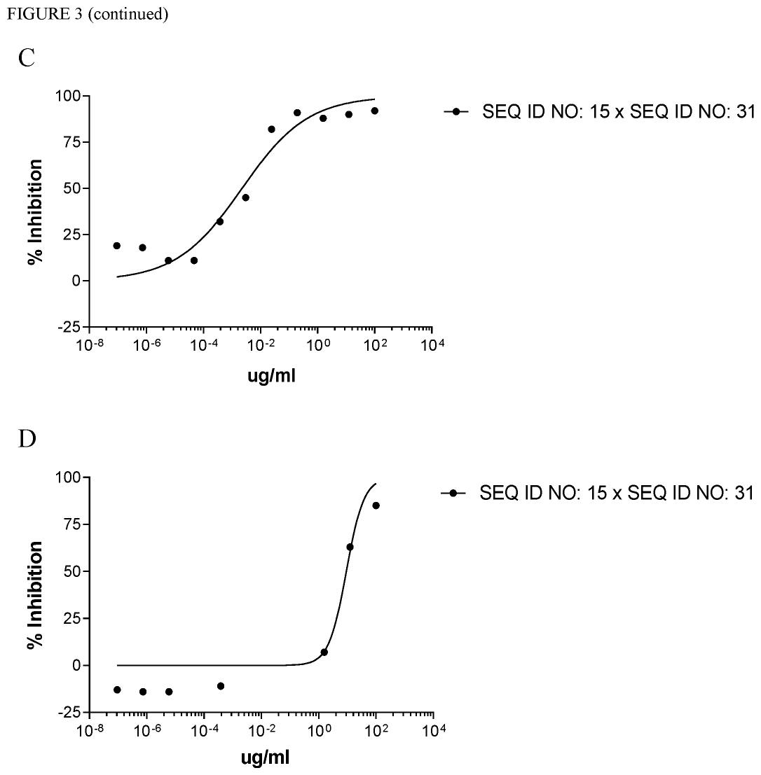

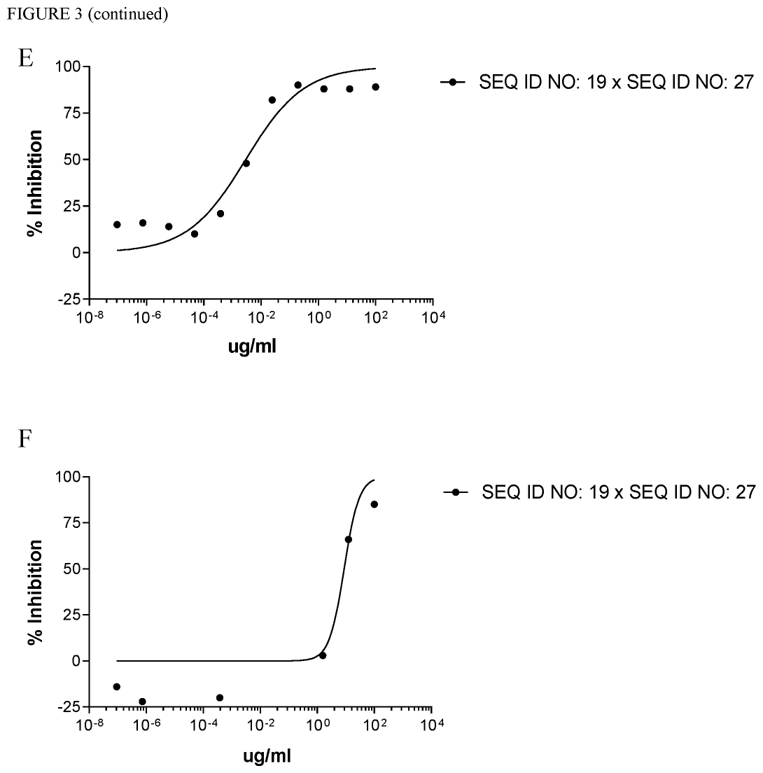

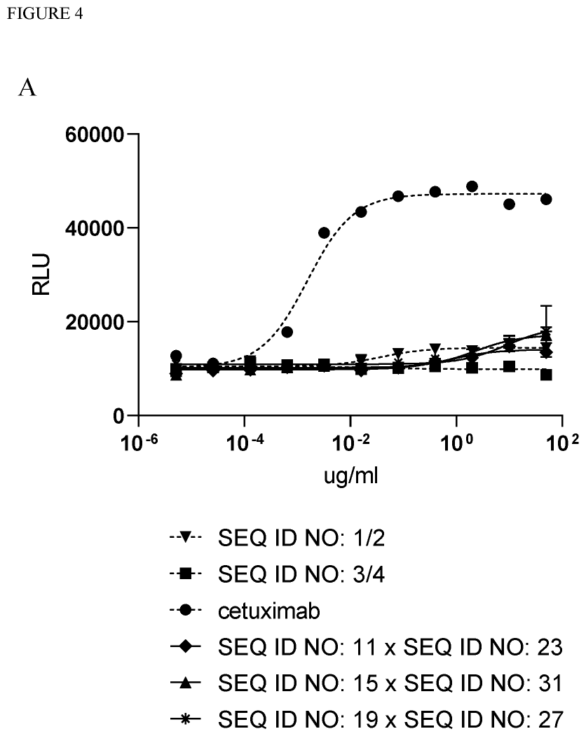

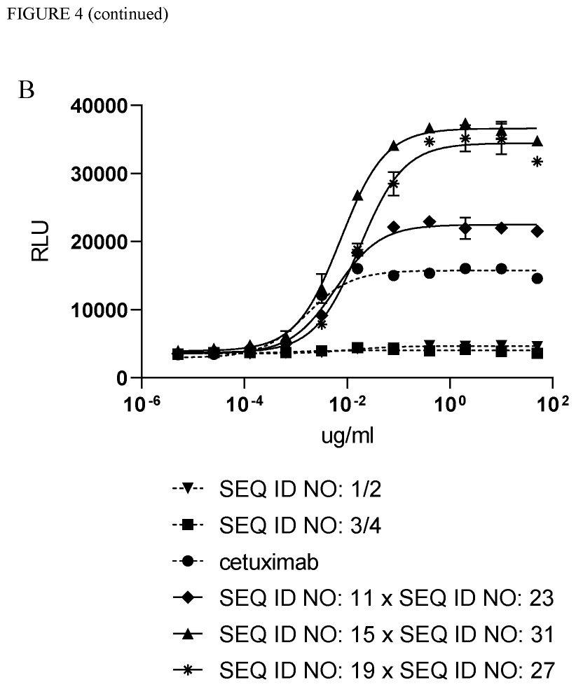

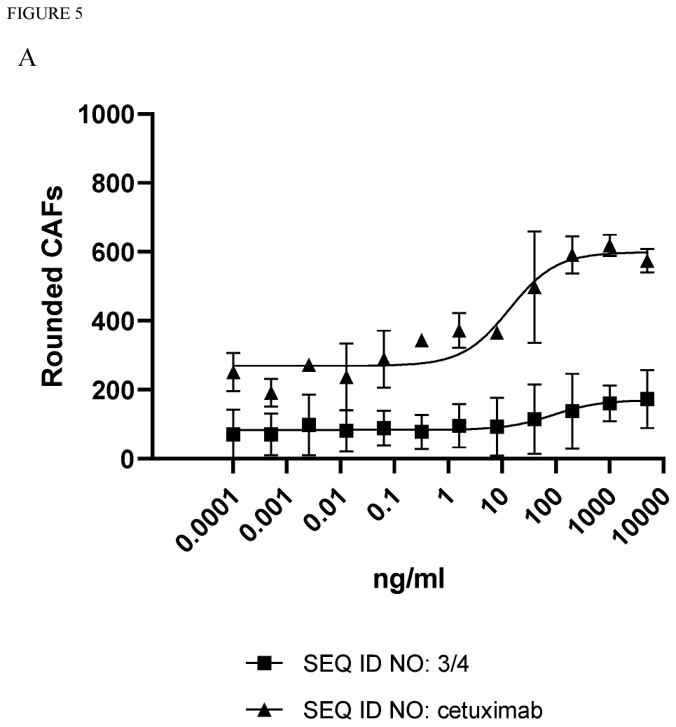

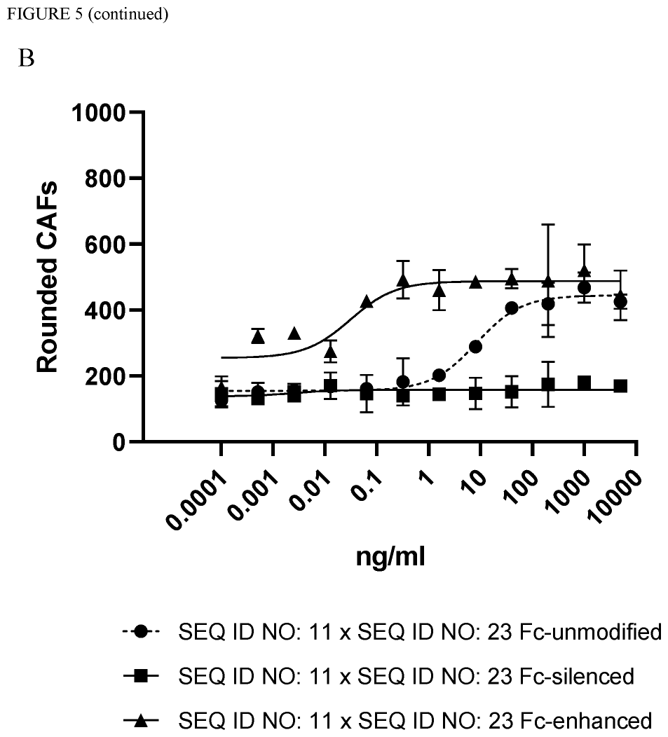

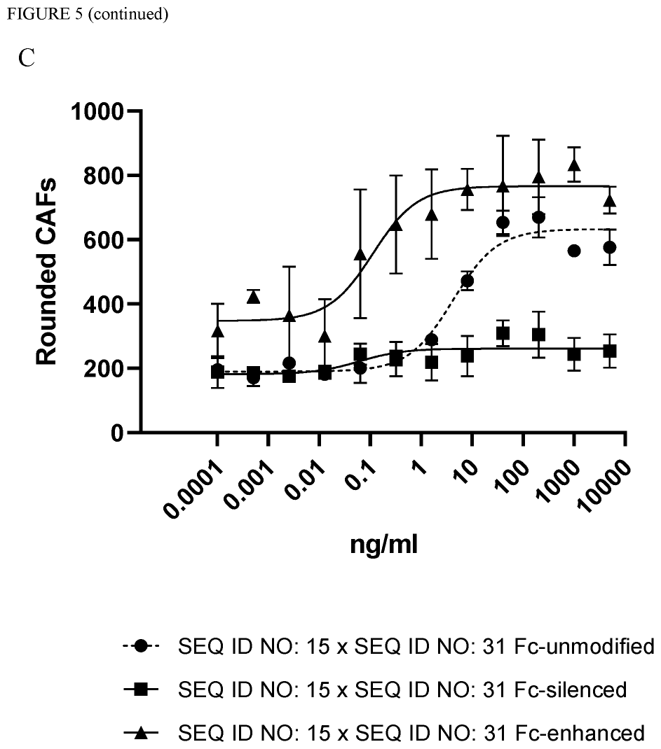

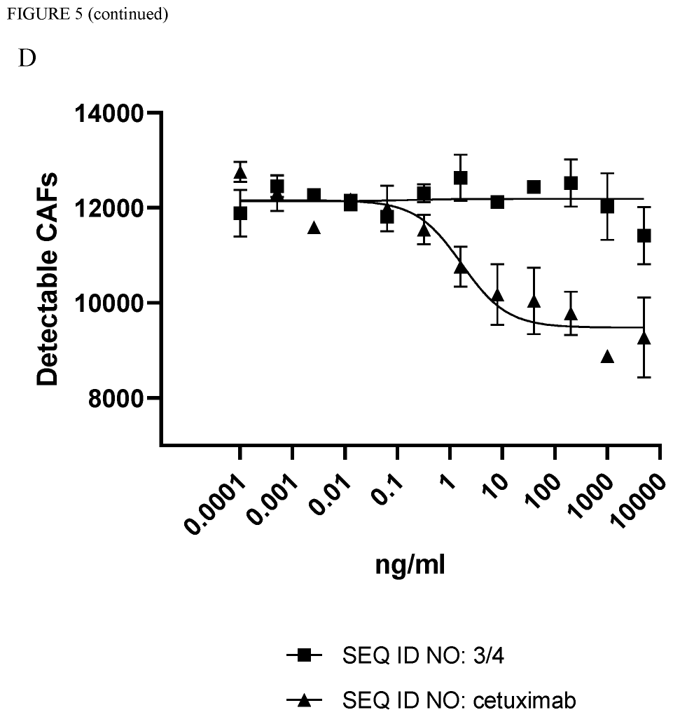

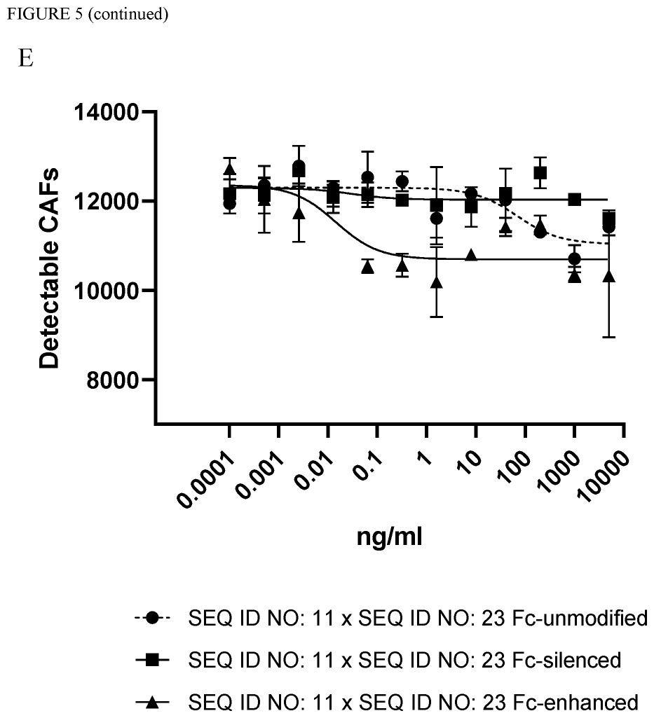

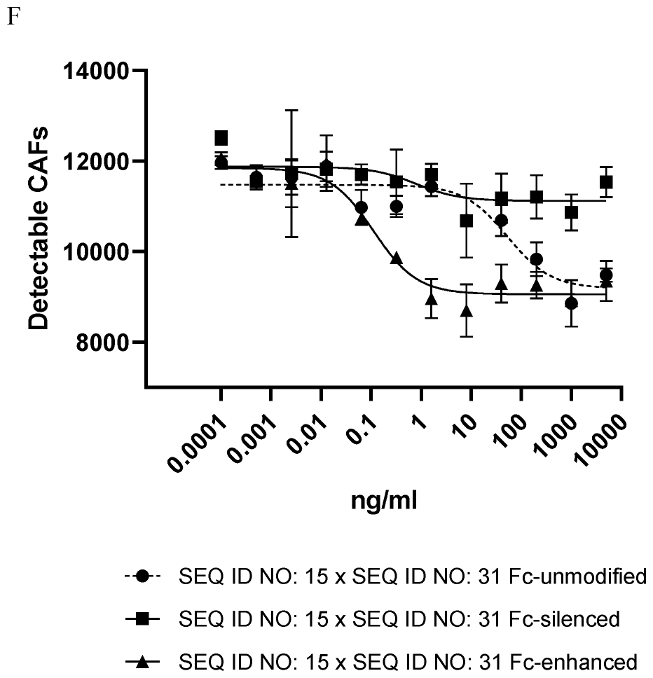

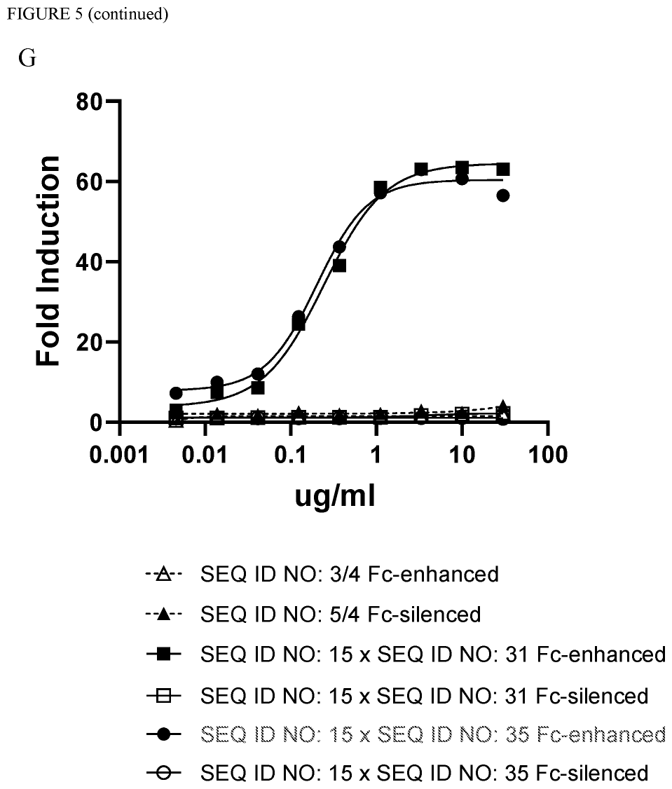

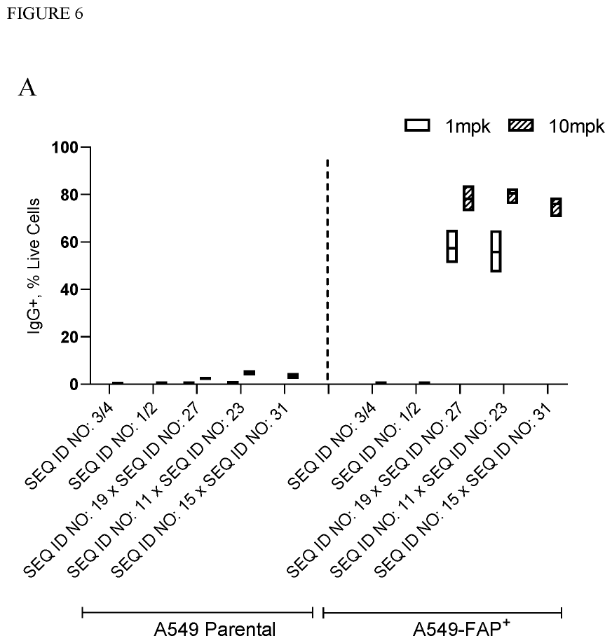

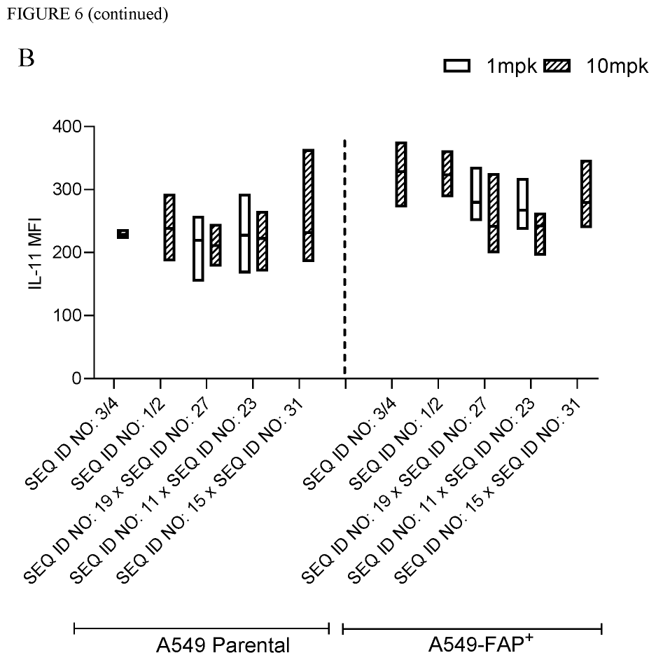

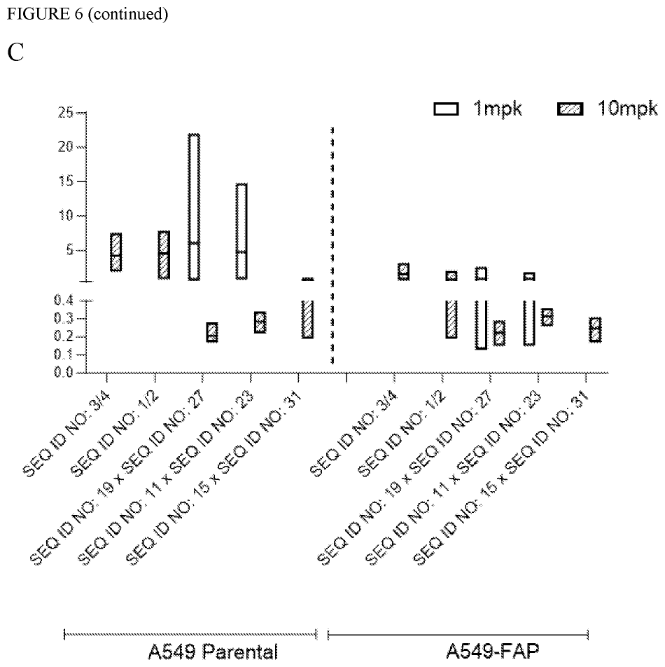

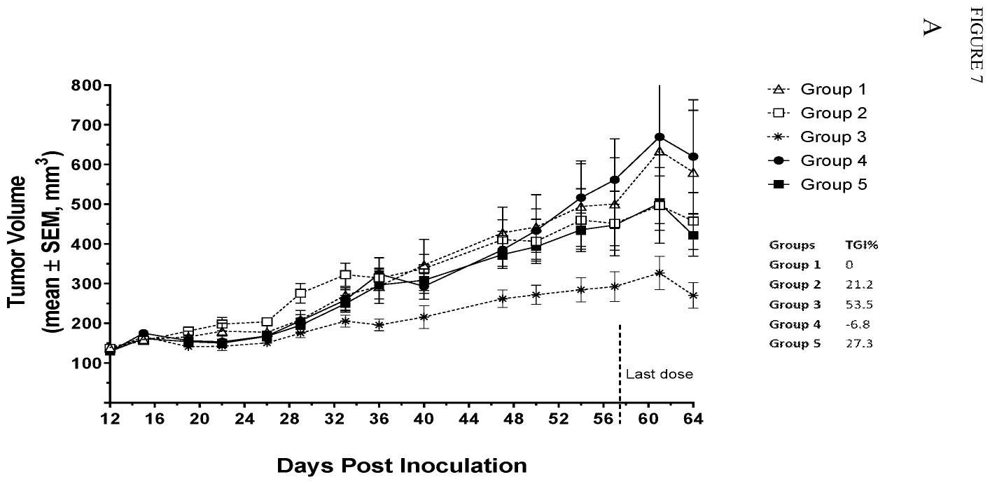

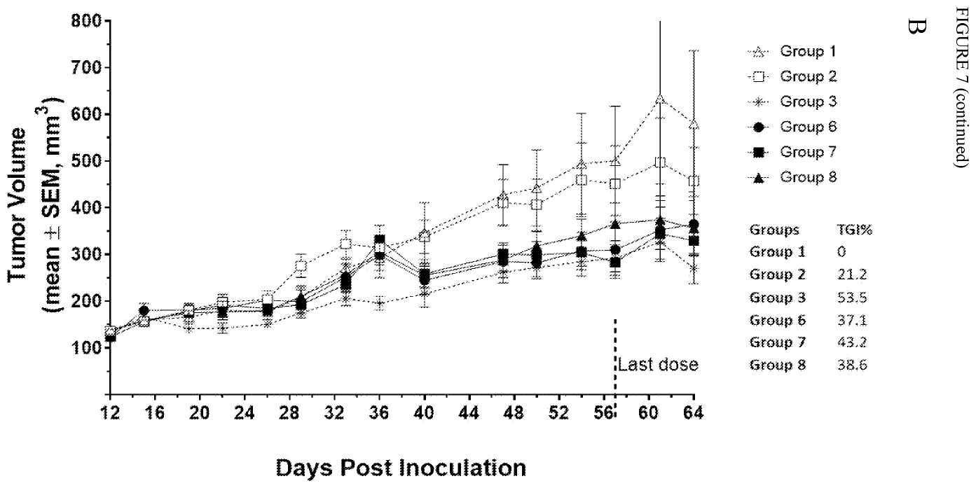

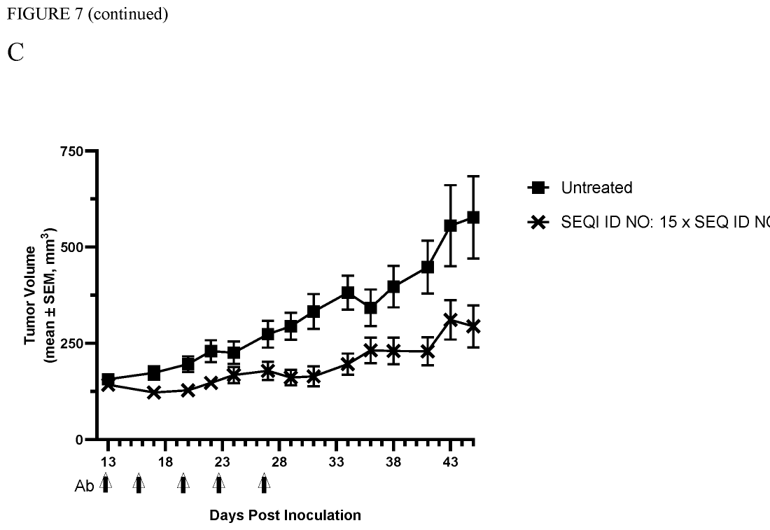

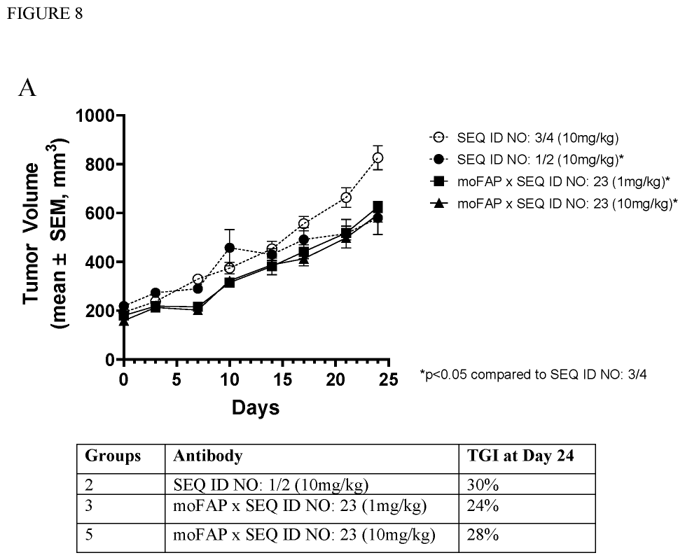

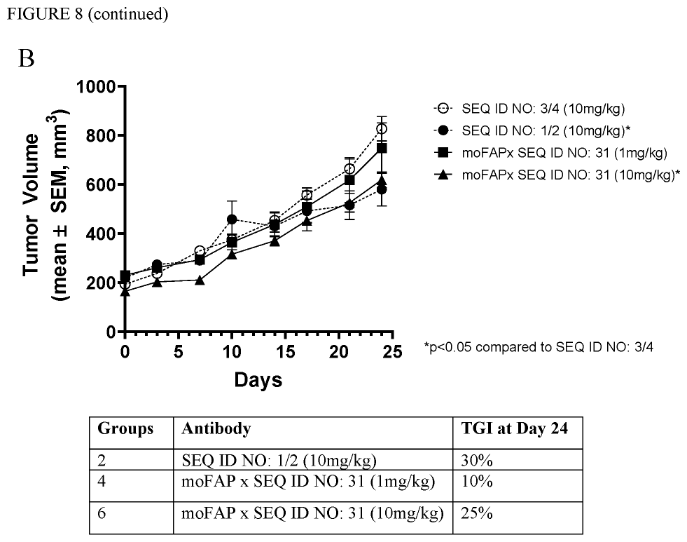

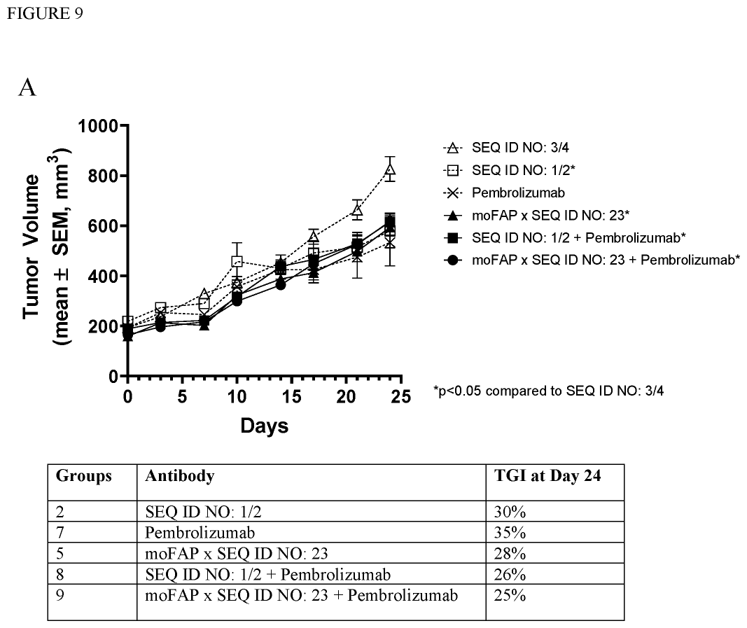

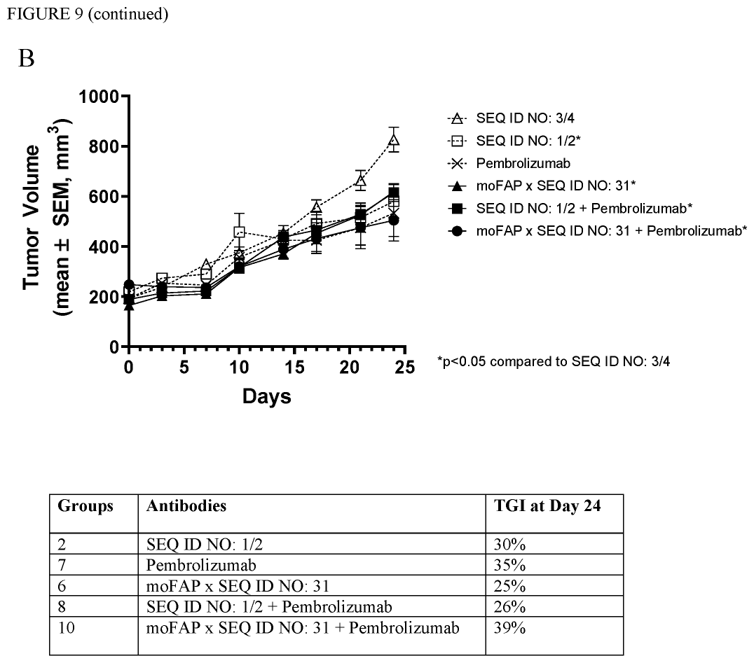

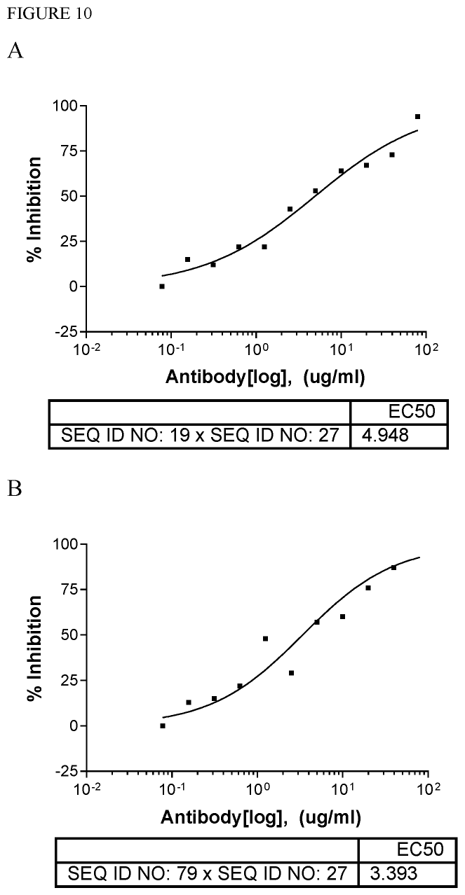

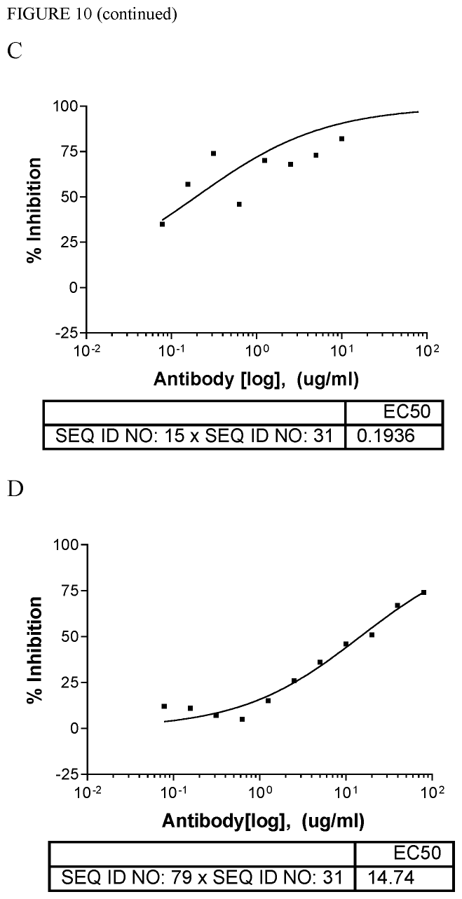

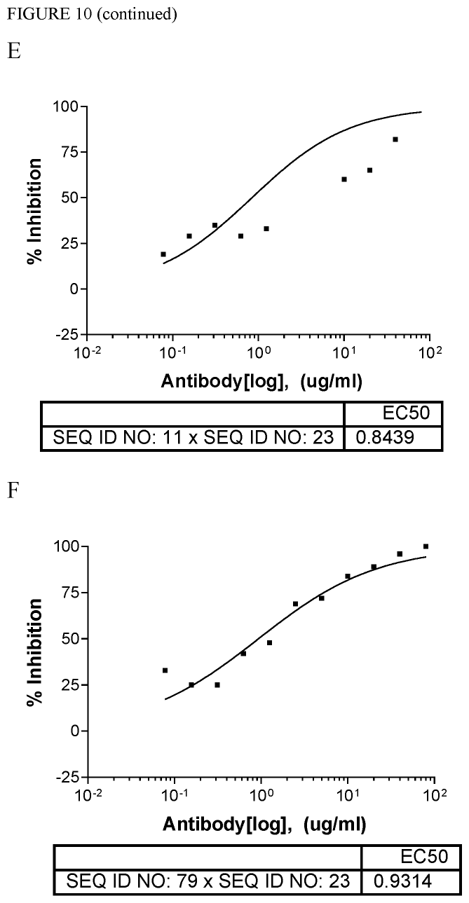

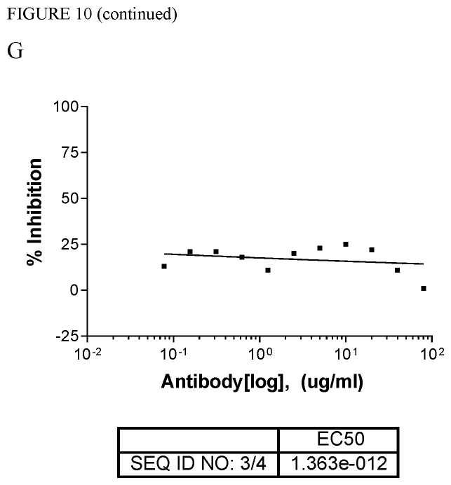

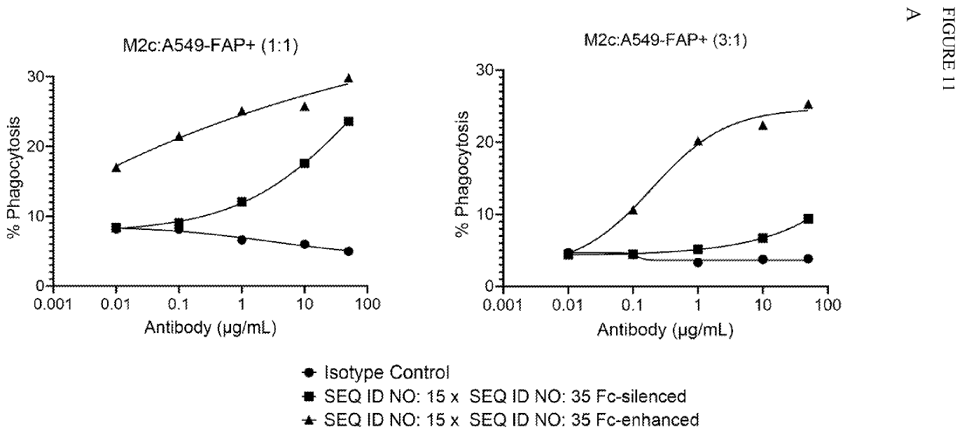

Results are shown in Table 4 and FIG. 2. All bispecific antibodies inhibited TGF-βRII mediated signaling. FIG. 2A shows the % inhibition of pSMAD2 expression and FIG. 2B shows the % inhibition of IL-11 expression in primary colon CAFs for some of the antibodies. Bispecific antibodies set out below are monovalent for binding to TGF-βRII and demonstrate superior inhibition of TGF-βRII signaling compared to the bivalent monospecific analog reference antibody TGF1. The range of IL-11 inhibition of the bispecific antibodies is between 2-2700 fold higher, when compared to the inhibition obtained with the analog reference antibody TGF1 in this assay. The range of pSMAD2 inhibition of the bispecific antibodies is between 2-300 fold higher, when compared to the inhibition obtained with the analog reference antibody TGF1 in this assay.

TABLE 4
Inhibition of TGF-βRII induced pSMAD2 and IL-11 expression
by FAP × TGF-βRII antibodies.

| | | Fold | | Fold |
|---|---|---|---|---|
| | | difference | | difference |
| | | wrt analog | | wrt analog |
| | pSMAD2 IC50 | reference | IL-11 IC50 | reference |
| Antibody | (ug/ml) | TGF1 | (ug/ml) | TGF1 |
| SEQ ID NO: 19 × | 0.00004 | 232.2 | 0.00002 | 2628.5 |
| SEQ ID NO: 27 | | | | |
| SEQ ID NO: 15 × | 0.00011 | 84.4 | 0.00004 | 1314.3 |
| SEQ ID NO: 31 | | | | |
| SEQ ID NO: 11 × | 0.00448 | 2.1 | 0.02515 | 2.1 |
| SEQ ID NO: 23 | | | | |
| analog reference | 0.009286 | — | 0.05257 | — |
| TGF1 | | | | |

In addition to primary colon CAFs, inhibition of TGF-βRII signaling by the bispecific antibodies was also tested in a variety of different primary CAF cell lines. Details of the different CAFs used is provided in Table 3. The FAP×TGF-βRII bispecific antibodies tested were those indicated with SEQ ID NO: 15×SEQ ID NO: 31 and SEQ ID NO: 11×SEQ ID NO: 23.

FAP×TGF-βRII bispecific antibodies effectively inhibited TGF-βRII mediated signaling expression in 10 different primary human CAF cell lines. FIG. 2C shows pSMAD2 inhibition and FIG. 2D shows IL-11 inhibition for CAFs from lung adenocarcinoma (LUAD). Table 5 shows IL-11 and pSMAD2 inhibition for all CAFs tested.

TABLE 5
Inhibition of TGF-βRII induced pSMAD2 and IL-11 expression by FAP × TGF-βRII antibodies.

| Antibody | BLDA | COAD(1) | COAD(2) | GBM | HNSC | LUAD | LUSC | MEL | OV | PAAD |
|---|---|---|---|---|---|---|---|---|---|---|
| | Primary CAF pSMAD IC50 (ug/ml) | | | | | | | | | |
| SEQ ID NO: | 0.138 | 0.104 | 0.057 | 0.019 | 6.271 | 0.032 | 0.015 | 0.956 | 0.379 | 0.018 |
| 11 × SEQ | | | | | | | | | | |
| ID NO: 23 | | | | | | | | | | |
| SEQ ID NO: | 0.018 | 0.010 | 0.011 | 0.010 | 0.041 | 0.007 | 0.004 | 0.009 | 0.023 | 0.007 |
| 15 × SEQ | | | | | | | | | | |
| ID NO: 31 | | | | | | | | | | |
| analog | 0.087 | 0.081 | 0.023 | 0.048 | 0.714 | 0.078 | 0.125 | 0.117 | 0.188 | 0.126 |
| reference | | | | | | | | | | |
| TGF1 | | | | | | | | | | |
| | Primary CAF IL-11 IC50 (ug/ml) | | | | | | | | | |
| SEQ ID NO: | 2.811 | 1.897 | 0.735 | 0.046 | NA | 1.582 | 0.050 | 0.122 | 0.016 | 0.055 |
| 11 × SEQ | | | | | | | | | | |
| ID NO: 23 | | | | | | | | | | |
| SEQ ID NO: | 0.015 | 0.032 | 0.011 | 0.003 | 0.057 | 0.037 | 0.011 | 0.007 | 0.004 | 0.028 |
| 15 × SEQ | | | | | | | | | | |
| ID NO: 31 | | | | | | | | | | |
| analog | 1.045 | 1.221 | 0.313 | 0.264 | 20.17 | 0.765 | 0.085 | 0.588 | 0.191 | 0.380 |
| reference | | | | | | | | | | |
| TGF1 | | | | | | | | | | |

Further bispecific antibodies indicated with SEQ ID NO: 15×SEQ ID NO: 31 Fc-silenced, SEQ ID NO: 15×SEQ ID NO: 31 Fc-enhanced, SEQ ID NO: 15×SEQ ID NO: 35 Fc-silenced and SEQ ID NO: 15×SEQ ID NO: 35 Fc-enhanced, were tested in a TGF-βRII mediated signaling inhibition assay from MSD (MSD, cat. no. K151E2R-2), using IL-11 as a read-out. Primary colon CAFs were used (BioIVT, lot 427650A1-6037, 373853A2-6020 passage 5) and the assay was performed as described above. The negative control IgG1 antibody (RSV-G) in Fc-silenced and Fc-enhanced formats was used as a control antibody; the analog reference FAP antibody sibrotuzumab in Fc-enhanced format and the analog reference TGF-βRII antibody TGF1 were used as reference antibodies. The control antibodies and the analog reference FAP antibody sibrotuzumab did not affect IL-11 expression in primary CAFs, thereby validating the assay.

All bispecific antibodies effectively inhibited TGF-βRII mediated signaling expression in primary colon CAF cells as shown in FIG. 2E. Similar results were obtained in lung and additional colon CAFs, as shown in Table 6.

TABLE 6
Inhibition of TGF-βRII induced IL-11 expression by FAP × TGF-βRII antibodies.

| | Colon CAF | Colon CAF | Lung CAF |
|---|---|---|---|
| | (COAD1) | (COAD2) | (LUSC) |
| Antibody | IC50 (ug/mL) | IC50 (ug/mL) | IC50 (ug/mL) |
| SEQ ID NO: 15 × SEQ ID | 0.006 | 0.010 | 0.034 |
| NO: 31 Fc -enhanced | | | |
| SEQ ID NO: 15 × SEQ ID | 0.010 | 0.010 | 0.020 |
| NO: 31 Fc -silenced | | | |
| SEQ ID NO: 15 × SEQ ID | 0.009 | 0.006 | 0.084 |
| NO: 35 Fc - enhanced | | | |
| SEQ ID NO: 15 × SEQ ID | 0.007 | 0.010 | 0.007 |
| NO: 35 Fc - silenced | | | |

### Example 8: Receptor Density of huFAP and huTGF-βRII

FAP×TGF-βRII bispecific antibodies were characterized in various assays to determine their ability to mediate their function correlated with FAP expression. For this purpose two cell lines were generated. Parental A549 cells were obtained from ATCC and designated A549 parental cells. These parental A549 cells were modified to stably express FAP (A549-FAP⁺ cells).

Receptor density of huFAP and huTGF-βRII was evaluated on A549 parental and A549-FAP⁺ cells using PE beads and flow cytometry. Both BD Quantibrite PE Beads and Bangs Quantum MESF PE beads were used to quantify receptor density, according to the manufacturer's instructions provided in the kit. Cells were thawed, washed, and counted using the Guava ViaCount assay on a Guava easyCyte instrument. Cells were plated evenly across all wells in Ultra low binding U bottom plates, for immediate antibody staining. Viability dye (BD FVS780, cat. no. 565388) was added to all the samples to discriminate between live and dead cells. FAP and hu TGF-βRII antibodies (described in example 6) conjugated to PE were used for cell staining. PE-conjugated isotype controls (Isotype mIgG1, Isotype hIgG1) were also used as negative controls.

TABLE 7
Receptor Density on A549 parental cells and A549-FAP⁺ cells.

| | | | Fold difference |
|---|---|---|---|
| | huFAP | huTGF-βRII | in FAP levels |
| A549 parental | 244 | 7267 | na |
| A549-FAP⁺ | 1098213 | 10994 | 4500.9 |

Expression levels of huFAP and huTGF-βRII is described in Table 7 for A549 parental cells and A549-FAP⁺ cells. A549-FAP⁺ cells express huFAP at a higher level compared to A549 parental cells with a fold difference of about 4500 fold.

### Example 9: Inhibition of TGF-βRII Mediated Signaling in Mixed Culture pSMAD2 Assay

FAP×TGF-βRII bispecific antibodies were characterized in a mixed culture pSMAD2 assay to determine their ability to block TGF-βRII mediated signaling in correlation to FAP expression. The mixed culture pSMAD2 assay involved a comparison of the inhibition of TGF-βRII signaling by the bispecific antibodies in A549 parental cells and A549-FAP⁺ cells. A549 parental cells express human TGF-βRII but no, or undetectable levels of, FAP; and A549-FAP⁺ cells are A549 cells engineered to overexpress human FAP.

A549 parental cells were cultured in DMEM+10% FBS and A549-FAP⁺ (Example 8) cells were cultured in DMEM+10% FBS supplemented with 5 ug/mL puromycin until 80-90% confluent. Cells were trypsinized, washed 3× in medium, trypsinized and centrifuged at 200 g for 10 min. Cells were then washed twice in PBS. A549-FAP⁺ cells were labeled with 1 uM CFSE by re-suspending cells in pre-warmed PBS and mixing with equal volume of warm 2 uM CFSE (BD Bioscience). The cells were incubated at 37° ° C. for 20 min with occasional mixing. The CFSE reaction was stopped by adding cold FBS/medium followed by centrifuging. Labeled cells were subsequently washed 3× in media. A549 parental and A549-FAP⁺ cells were then mixed 1:1 and 50 μl transferred into 96-well at 1.2×10⁵ cells/well.

Antibodies were serially diluted 5-fold and added to wells starting at 400 ug/ml (4×) in 11 concentration-points. 50 μl of antibodies was added to wells, mixed and incubated at room temperature for 1 hour. 100 μl of 2 ng/ml (2×) of recombinant human TGF-β1 was added to wells for 2 hours. Cells were washed in PBS and re-suspended in 50 μl of 300-fold diluted viability dye (Zombie Violet, BioLegend #423113) in 1% FBS/PBS for 10 min. Cells were then washed, fixed and permeabilized according to BD TFP protocol using 200 μl of buffer. Cells were stained with anti-pSMAD2 antibody (Cell Signaling #E8F3R) on ice for 1 hour. Cells were then washed and stained with anti-rabbit IgG secondary antibody (JAX #611-605-215) for 30 minutes on ice in the dark. Following staining, cells were washed 3× in wash buffer, re-suspended in 200 μl of PBS, and cells were acquired with a Fortessa flow cytometer.

Results are shown in Table 8 and FIG. 3. Table 8 shows IC50 values and the fold difference in potency in inhibiting TGF-βRII signaling in A549 parental versus A549-FAP⁺ cells for exemplary bispecific antibodies. The fold difference of pSMAD2 inhibition for the bispecific antibodies in A549-FAP⁺ cells is between 600-19000 fold higher than the fold difference in A549 parental cells in this assay.

All bispecific antibodies inhibited TGF-βRII mediated signaling in both A549 parental cells and A549-FAP⁺ cells. The bispecific antibodies are more potent in inhibiting TGF-βRII signaling in A549-FAP⁺ cells than in A549 parental cells, indicating that they inhibit TGF-β-induced SMAD2 signaling in a manner correlated to FAP expression.

The results of the mixed culture pSMAD2 assay indicate that the specificity of the bispecific antibodies to FAP⁺ cells is by the FAP binding domain. This is further supported by the in vivo tumor targeting study described in Example 12.

TABLE 8
Inhibition of TGF-βRII induced pSMAD2 expression by FAP × TGF-βRII
antibodies in A549 parental and A549-FAP⁺ cells.

| | | | | | Parental |
|---|---|---|---|---|---|
| | | | | A549 | FAP/FAP+ |
| | | | A549-FAP+ | Parental | cells IC50 |
| Antibody | TGFBRII arm | FAP arm | cells IC50 | IC50 | Ratio |
| 1 | SEQ ID NO: 27 | SEQ ID NO: 19 | 0.00055 | 10.28 | 18606 |
| 2 | SEQ ID NO: 31 | SEQ ID NO: 15 | 0.00366 | 12.17 | 3327 |
| 3 | SEQ ID NO: 23 | SEQ ID NO: 11 | 0.0209 | 14.33 | 686 |

### Example 10: Killing of FAP⁺ Cells: ADCC Assay

An ADCC assay was performed to test the ability of bispecific antibodies to mediate killing of antibody-coated target cells by immune effector cells via Fc receptors that recognize the constant region of the antibodies.

Several bispecific antibodies were tested in unmodified format in the ADCC assay. Technical controls of the assay were negative control RSV-G antibody and cetuximab. Target cells A549 parental and A549-FAP⁺ cells were obtained as described in Example 8.

For detection of ADCC, the KILR® detection kit (#97-0001M) from Eurofins was used. In this assay, A549-FAP⁺ and A549 parental cells were engineered to express a protein with an enhanced ProLabel, a β-Gal reporter fragment. When target cells are killed by CD16 effector cells, the reporter fragment can be detected in the supernatant using the enzyme acceptor fragment of the β-Gal enzyme. CD16 KILR effector cells were grown in AssayComplete Cell culture media supplemented with 600 IU/ml of recombinant human IL-2 according to Eurofins protocol. Cells were fed with fresh media and 600 IU/ml recombinant human IL-2 every 2 days. A549 parental cells were grown in DMEM supplemented with 10% HI-FBS, 1× glutamine and 500 ug/mL G418. A549-FAP⁺ target pool cells were grown in DMEM supplemented with 10% HI-FBS, 1× glutamine, 5 ug/mL puromycin, and supplemented with 500 ug/mL G418. Once A549-FAP⁺ cells were established, they were maintained in media containing 500 μg/ml of G418. For the experiment, A549-FAP⁺ or A549 parental target cells were harvested following having been cultured in antibiotic-free media for 48 hours. Cells were washed twice with media and re-suspended in media at 200,000 cells/mL. Target cells were seeded in 50 μL of medium for 30 min. Antibodies were added to target cells and incubated for 30 minutes at 37° C. and 5% CO₂. CD16 effector cells were re-suspended to a concentration of 1.6×10⁶/mL, and added to the target cells at an E:T ratio of 10:1. Cells were incubated for 3 hr at 37° ° C. and 5% CO₂. KILR detection solution was added and incubated for 1 hour at room temperature in the dark. Following incubation, chemiluminescent signal was detected.

Results are shown for exemplary antibodies in FIG. 4 and Table 9. FAP×TGF-βRII bispecific antibodies exhibit high ADCC killing activity in A549-FAP⁺ cells as compared to A549 parental cells. The ADCC activity of FAP×TGF-βRII bispecific antibodies was higher compared to the ADCC activity of cetuximab.

TABLE 9
ADCC activity of FAP × TGF-βRII antibodies
in A549-FAP⁺ cells.

| | CD16 A549-FAP KILR |
|---|---|
| Antibody | ADCC EC50 (ug/ml) |
| SEQ ID NO: 11 × SEQ ID NO: 23 | 0.0082 |
| SEQ ID NO: 15 × SEQ ID NO: 31 | 0.0045 |
| SEQ ID NO: 19 × SEQ ID NO: 27 | 0.0156 |
| cetuximab | 0.0004 |

### Example 11: ADCC Activity Mediated by Different Fc Formats

FAP×TGF-βRII bispecific antibodies were tested in Fc-silenced, unmodified, and Fc-enhanced IgG1 format to compare the effect of different Fc backbones in mediating ADCC killing. Bispecific antibodies indicated with SEQ ID NO: 15×SEQ ID NO: 31 and SEQ ID NO: 11×SEQ ID NO: 23 were tested for ADCC activity using primary CAF cells as target cells (melanoma CAFs and lung adenocarcinoma CAFs as described in Example 6) and human NK cells as immune effector cells. Technical controls of the assay were negative control RSV-G antibody and positive control cetuximab, both in unmodified Fc format.

Two days prior to co-culture with CAFs, human NK cells were thawed, washed and re-suspended in RPMI+10% FBS with 50 IU/ml of rhIL-2 for 2 days. On the day of co-culture, NK cells were centrifuged, washed, and re-suspended at 2×10⁶/mL in phenol red free assay media. CAFs were grown to ˜90% confluency in a T150 flask. CAFs were washed and stained with complete media containing Nuclight Rapid Red reagent at 1:500 dilution in a total of 20 mL. CAFs were allowed to incubate at 37° C. and 5% CO₂ overnight. The following morning, cells were washed with PBS, trypsinized, and re-suspend at 1×10⁵/mL in phenol red free assay media. The morning of the assay setup 100 μl of the stained CAFs were added to each well of a 96 well flat bottom plate. Plates were incubated for 30 min at RT, then for 6 hours at 37° C. and 5% CO₂. Antibody dilutions were prepared in a 12 concentration-point curve in phenol red free media. Following 6 hours, 50 μl of NK cells and 50 uL of antibody dilutions were added to each appropriate well. Plates were incubated for 30 min at room temperature. Plates were scanned using the Incucyte instrument in phase contrast and red channels at 3 hr interval using the ‘adherent cell by cell module’, and stopped after 15 hr. For the melanoma CAFs, cells that were visibly dead with retained red stain were counted. For the lung adenocarcinoma CAFs, cells that were visibly viable were counted. NK cells were excluded from the cell count by size filtering. Live or dead cell numbers were plotted against antibody concentrations, and EC50 calculated for live cells in GraphPad Prism. FIG. 5 shows the killing activity of the antibodies either as rounded CAFs per well as an indication that killed CAFs detach from the adherent surface (A-C) or as detectable CAFs (D-F).

Results are shown in Table 10 and FIGS. 5A-F. FAP×TGF-βRII bispecific antibodies indicated with SEQ ID NO: 15×SEQ ID NO: 31 and SEQ ID NO: 11×SEQ ID NO: 23 exhibit ADCC killing activity in different primary human CAF cell lines. Both antibodies were more potent in ADCC killing when used in an enhanced-Fc format, compared to unmodified Fc format.

TABLE 10
ADCC activity of bispecific antibodies in Fc-enhanced and unmodified
IgG1 format using primary CAF target and NK effector cells.

| | | | ADCC EC50 (ng/mL) | | | | |
|---|---|---|---|---|---|---|---|
| | | | | SEQ ID | SEQ ID | SEQ ID | SEQ ID |
| | | | | NO: 11 × | NO: 11 × | NO: 15 × | NO: 15 × |
| | Receptor | | | SEQ ID | SEQ ID | SEQ ID | SEQ ID |
| CAF | Density | | | NO: 23 - | NO: 23 - | NO: 31 - | NO: 31 - |
| Origin | TGFBR2 | FAP | Cetuximab | unmodified | Enhanced Fc | unmodified | Enhanced Fc |
| LUAD | 5-10k | 25-50k | 1.674 | 68.53 | 0.01451 | 53.05 | 0.1121 |
| MEL | 10-15k | 100-300k | 13.72 | 8.341 | 0.03117 | 4.187 | 0.113 |

A further ADCC reporter assay was performed with the following antibodies: SEQ ID NO: 15×SEQ ID NO: 31 Fc-silenced, SEQ ID NO: 15×SEQ ID NO: 31 Fc-enhanced, SEQ ID NO: 15×SEQ ID NO: 35 Fc-silenced and SEQ ID NO: 15×SEQ ID NO: 35 Fc-enhanced. Negative control RSV-G antibody in Fc enhanced and Fc silenced formats were used as reference antibodies.

A Promega ADCC reporter bioassay kit (cat. no. G7018) was used and the assay was performed according to the manufacturer's protocol. Briefly, colon CAFs were plated at 20,000 cells in 100 uL/well in a 96-well plate and incubated overnight. The next day 25 μl assay buffer/well was added. Antibodies were then added at 25 μl/well starting at 10 ug/ml with a serial dilution of 1:5 resulting in the following ug/ml concentrations (10, 2, 0.4, 0.08, 0.016, 0.0032, 0.00064, 0.000128, 0.0000256). FcγRIIIa effector cells were thawed and immediately added to the CAFs at 7.5×10⁴ cells in 25 uL/well. The plates were covered and incubated for 6 hours at 37° C. in a humidified CO₂ incubator. The plates were then equilibrated to ambient temperature for 15 minutes. Bio-Glo Luciferase Assay Reagent was added at 75 μl/well. The plates were incubated for 20 minutes. Luminescence was measured using a plate reader. Fold of induction was calculated by the following:

$$Fold of Induction = RLU ( induced - background ) / RLU ( no antibody ctrl - background )$$

Results are shown in FIG. 5G and Table 11. The negative control antibody RSV-G-Fc enhanced and negative control antibody RSV-G-Fc silenced did not mediate ADCC killing of primary CAFs, thereby validating the assay. Fc-enhanced bispecific antibodies effectively mediated ADCC killing as compared to Fc-silenced bispecific antibodies.

TABLE 11
EC50 values for ADCC activity of Fc-Enhanced antibodies.

| Antibody | EC50 (nM) |
|---|---|
| SEQ ID NO: 15 × SEQ ID NO: 31 Fc Enhanced | 0.24 |
| SEQ ID NO: 15 × SEQ ID NO: 35 Fc Enhanced | 0.20 |

### Example 12: FAP×TGF-βRII Antibody Mediated In Vivo Tumor Targeting

Bispecific antibodies were characterized in vivo in an NSG mouse model to determine their ability to selectively localize and inhibit TGF-βRII mediated signaling in tumor cells expressing both FAP and TGF-βRII. For this purpose, NSG mice were inoculated with either A549 parental or A549-FAP⁺ tumor cells and treated with the bispecific antibodies. The mouse model was validated using the following antibodies: negative control RSV-G antibody, experimental control FAP×TGF-βRII antibody and analog reference antibody TGF1 (data not shown).

Several bispecific antibodies were tested in Fc-silenced or Fc unmodified formats.

Approximately 5 million A549 parental or A549-FAP⁺ cells were re-suspended in 200 ul in a 1:1 mix of PBS and Matrigel (VWR cat. no. 47743-706), and inoculated into the flank of NSG mice. After tumors were established, the mice were randomized by body weight into the following treatment groups:
1) Negative control RSV-G antibody 10 mg/kg
2) Analog reference TGF1 antibody 10 mg/kg
3) SEQ ID NO: 19×SEQ ID NO: 27 1 mg/kg
4) SEQ ID NO: 19×SEQ ID NO: 27 10 mg/kg
5) SEQ ID NO: 11×SEQ ID NO: 23 1 mg/kg
6) SEQ ID NO: 11×SEQ ID NO: 23 10 mg/kg
7) SEQ ID NO: 15×SEQ ID NO: 31 1 mg/kg
8) SEQ ID NO: 15×SEQ ID NO: 31 10 mg/kg

Bispecific antibodies, control, and reference antibodies were administered on Day 17 and Day 20 after cell inoculation. Mice were dosed on days 3 and 4, and sacrificed and tumors collected on day 5 post-inoculation.

Tumors were collected and placed on ice. Single cells were made by mashing tumors in a 100 micron filter using 3 ml syringe plunger into 50-ml falcon tubes. Filters were rinsed with DMEM+10% FBS into Falcon tubes to drain single cells into tubes. The cells were then pelleted by centrifuging at 300 g for 10 min. Cell pellets were washed 1× in 1% FBS/PBS FACS media and counted. 1-2 million cells were transferred into a 96-well plate and stained in 100 μl of diluted surface primary antibodies for 30 min on ice in FACS buffer protected from light.

For IL-11 stain, cells were incubated in media containing 1× monensin (eBioscience™, cat. no. 00-4505-51, ThermoFisher Scientific) for 5 hr before FACs staining. The cells were then washed 3× in FACS buffer by centrifuging. For pSMAD2 staining (Cell Signaling, Cat no. E8F3R), cells were washed, fixed and permeabilized according to BD TFP protocol (cat. no. 563239) using 200 μl of buffer. For IL-11 staining (Thermo Fisher, cat. no. 551691AP), cells were fixed/permeabilized according to Ebioscience Foxp3 protocol (Thermo Fisher, cat. no. 00-5523-00) using 200 μl of buffer. For human IgG staining, antibody from Jackson Laboratories (cat. no. 109-605 003) was used. The cells were then re-suspended in 200 μl of 400-fold primary antibody and stained on ice in the dark for 1 hr. The cells were washed 2× with wash buffer and subsequently stained with 400-fold diluted secondary antibodies for 30 min on ice in the dark. Following staining, cells were washed 3× in wash buffer, re-suspended in 200 μl of PBS and acquired in a BD Fortessa flow cytometer.

Results are shown in FIG. 6. Data is not shown for pSMAD2 staining.

FIG. 6A shows IgG staining of tumor cells isolated from mice inoculated with A549 parental vs A549-FAP⁺ cells. All FAP×TGF-βRII bispecific antibodies preferentially localized to A549-FAP⁺ tumors compared to A549 parental tumors when administered at 1 mg/ml (mpk) and 10 mg/ml (mpk). This is in contrast to the analog reference TGF1 antibody.

FIG. 6B shows inhibition of TGF-βRII induced IL-11 expression in tumor cells. FAP×TGF-βRII bispecific antibodies effectively reduced IL-11 levels in mice inoculated with A549-FAP⁺ tumor cells when compared to A549 parental cells. The extent of IL-11 inhibition was greater in mice harboring A549-FAP⁺ tumors and treated with bispecific antibodies as compared to IL-11 inhibition in mice harboring A549-FAP⁺ tumors and treated with analog reference TGF1 antibody TGF1. Negative control RSV-G antibody did not suppress IL-11 expression in this assay.

FIG. 6C shows inhibition of TGF-βRII induced pSMAD2 expression in tumor cells. FAP×TGF-βRII bispecific antibodies effectively reduced pSMAD2 levels in mice inoculated with A549-FAP⁺ tumor cells when compared to A549 parental cells. The extent of pSMAD2 inhibition was greater in mice harboring A549-FAP⁺ tumors and treated with bispecific antibodies as compared to pSMAD2 inhibition in mice harboring A549-FAP⁺ tumors and treated with analog reference TGF1 antibody TGF1. Negative control RSV-G antibody did not suppress pSMAD2 expression in this assay.

Taken together, the results indicate that FAP×TGF-βRII bispecific antibodies selectively localize to tumors expressing both FAP and TGF-βRII in an in vivo mouse model and inhibit TGF-βRII mediated signaling in manner correlated with FAP expression.

Bispecific antibodies comprising a FAP binding domain comprising a heavy chain variable region having an amino acid sequence as set forth in SEQ ID NO: 15, SEQ ID NO: 19; or SEQ ID NO: 11, thus show selective functional activity towards cells expressing both FAP and TGF-βRII as compared to cells expressing TGF-βRII but undetectable levels of FAP. These FAP binding domains, and variants thereof, therefore are useful for the development of FAPxTGF-βRII bispecific antibodies for the treatment of cancer.

### Example 13: FAP×TGF-βRII Antibody Mediated In Vivo Tumor Efficacy

Bispecific antibodies were characterized in vivo in an athymic nude mice model bearing A549-FAP⁺ tumors, to determine their potency in reducing tumor volume.

Female BALB/c nu/nu, (5 to 7 weeks of age, Charles River Laboratories or Taconic Biosciences) were inoculated with 1×10⁷ tumor cells (A549-FAP⁺ cells, described in Example 8) and matrigel (BD Biosciences #354234) in 0.2 mL sterile PBS. The inoculation was performed subcutaneously on the flank. The treatment of tumor bearing mice was started 12 or 13 days post cell inoculation with average tumor volume of 135 mm³ or 143 mm³. Experimental therapeutic agents were administered to mice by intraperitoneal injection (IP). Cetuximab (Erbitux, NDC: 66733-958-23; LOT #C2100112) was purchased from RefDrug. Treatment frequency was two times a week for 7 or 5 weeks for this study. Treatment groups included are as set out in Table 12:

TABLE 12
Treatment groups for evaluation of TGF-βRII bispecific
antibodies mediated in vivo tumor cell killing in athymic
nude mice bearing A549-FAP⁺ tumors.

| | | Fc effector | | |
|---|---|---|---|---|
| | Antibody | function | N/group | Does |
| Group 1 | Negative control RSV-G | Enhanced | 10 | IP 2×/wk |
| | antibody- 30 mg/kg | | | |
| Group 2 | Analog reference TGF1 | Unmodified | 10 | IP 2×/wk |
| | antibody - 30 mg/kg | | | |
| Group 3 | Positive control | Unmodified | 10 | IP 2×/wk |
| | cetuximab - 30 mg/kg | | | |
| Group 4 | Analog reference | Enhanced | 10 | IP 2×/wk |
| | Sibrotuzumab- 3 mg/kg | | | |
| Group 5 | Analog reference | Enhanced | 10 | IP 2×/wk |
| | Sibrotuzumab - 30 mg/kg | | | |
| Group 6 | SEQ ID NO: 15 × SEQ ID | Enhanced | 10 | IP 2×/wk |
| | NO: 31 - 3 mg/kg | | | |
| Group 7 | SEQ ID NO: 15 × SEQ ID | Enhanced | 10 | IP 2×/wk |
| | NO: 31 - 30 mg/kg | | | |
| Group 8 | SEQ ID NO: 15 × SEQ ID | Unmodified | 10 | IP 2×/wk |
| | NO: 31 - 30 mg/kg | | | |
| Group 9 | SEQ ID NO: 15 × SEQ ID | Silenced | 8 | IP 2×/wk |
| | NO: 35 - 30 mg/kg | | | |

The size of subcutaneous tumors was measured two times weekly using a digital caliper. Tumor volume was calculated by measuring the tumor in 2 dimensions and using the equation: Volume=[Length×(Width2)]/2; where the larger number was length, and the smaller number was width. The effects on tumor growth were reported as percent tumor growth inhibition (% TGI), which was calculated using the equation: (1−(Tx vol./control vol.))*100, where control volume was the vehicle or untreated tumor. No significant weight loss was seen in all the treatment groups (data not shown).

Results are shown in FIG. 7. The bispecific antibody indicated with SEQ ID NO: 15×SEQ ID NO: 31 induced an anti-tumor response which was comparable to the anti-tumor response elicited by positive control antibody cetuximab (FIG. 7B). The bispecific antibody indicated with SEQ ID NO: 15×SEQ ID NO: 31 (Fc-Enhanced) at 3 mg/kg was as potent as the antibody indicated with SEQ ID NO: 15×SEQ ID NO: 31 (Fc unmodified) at 30 mg/kg in inducing an anti-tumor response (FIG. 7B). The bispecific antibody indicated with SEQ ID NO: 15×SEQ ID NO: 35 (Fc-silenced) induced higher in vivo tumor reduction in comparison to the untreated mice (FIG. 7C).

### Example 14: Binding Affinity

Binding affinity of bispecific antibodies comprising FAP binding domain according to SEQ ID NO: 15 and TGF-βRII binding domain according to SEQ ID NO: 31, was determined for binding to human FAP and to human TGF-βRII. SPR experiments were run on a Biacore T200 controlled by T200 control software.

All test antibodies were captured on a CM5 chip surface by an immobilized anti-human IgG antibody (Biacore®), followed by addition of human TGF-βRII (R&D, cat. no. 241-R2/CF) or human FAP (R&D, cat. No. 3715-SE). Measurements were carried out at 25° C. Binding data was analyzed using Biacore T200 Evaluation Software with double-reference subtraction (0 concentration and reference flow cell 1 with no antibody capture).

Results are shown in Table 13 for the exemplary antibodies tested. Affinity of the FAP binding domain in the FAP×TGF-βRII bispecific antibodies is in the range of 0.1-0.2 nM. Affinity of TGF-βRII binding domain in the FAP×TGF-βRII bispecific antibodies is in the range of 3.8-5 nM. As shown in Table 13, affinity of the TGF-βRII binding domain is in the range of 25-50 fold lower compared to the affinity of the FAP binding domain.

TABLE 13
SPR based binding kinetics of bispecific antibodies.

| | FAP binding | | | TGFBR2 binding | | | Fold KD |
|---|---|---|---|---|---|---|---|
| | ka | kd | KD | ka | kd | KD | (TGF- |
| Antibody | (1/Ms) | (1/s) | (M) | (1/Ms) | (1/s) | (M) | βRII/FAP) |
| SEQ ID NO: 15 × | 3.23E+05 | 4.04E−05 | 1.25E−10 | 4.45E+05 | 1.73E−03 | 3.88E−09 | 31.04 |
| SEQ ID NO: | | | | | | | |
| 31 Fc-silenced) | | | | | | | |
| SEQ ID NO: 15 × | 3.58E+05 | 3.61E−05 | 1.01E−10 | 3.98E+05 | 1.77E−03 | 4.44E−09 | 43.96 |
| SEQ ID NO: | | | | | | | |
| 31 Fc- enhanced | | | | | | | |
| SEQ ID NO: 15 × | 2.77E+05 | 5.03E−05 | 1.82E−10 | 3.35E+05 | 1.69E−03 | 5.03E−09 | 27.64 |
| SEQ ID NO: | | | | | | | |
| 35 Fc-silenced | | | | | | | |
| SEQ ID NO: 15 × | 3.05E+05 | 3.55E−05 | 1.16E−10 | 4.37E+05 | 1.81E−03 | 4.15E−09 | 35.78 |
| SEQ ID NO: | | | | | | | |
| 35 Fc- enhanced | | | | | | | |

### Example 15: TGF-β Reporter Assay—Trans TGF-βRII Inhibition Assay

Bispecific antibodies were characterized in a TGF-β reporter assay to determine their ability to inhibit TGF-βRII mediated signaling by trans-mode activity (trans binding) on HEKBlue-TGF-βRII reporter cells (HEK-Blue cells). The assay was performed using a coculture of MRC5 cells which were obtained from ATCC (#CCL171) and validated for expression of FAP and TGF-βRII, and HEKBlue cells, which were purchased from In Vivogen (#hkb-tgfbv2) known to express TGF-βRII.

Recombinant human TGF-β1 (rhTGF-β1; R&D #240-B) was prepared in 2-fold serial dilutions in media containing 0.1% FBS in duplicates with 40 ng/ml (2×) initial concentration. Eighty percent confluent HEK-Blue and MRC-5 cells were trypsinized, washed 2λ, re-suspended to 2×10⁶/ml, mixed 1:1 and transferred 25 μl (25,000 cells)/well.

Serially diluted bispecific antibodies were added to the mixture of cells, mixed gently and incubated at room temperature for 2 hours. Then, 100 μl of diluted of rhTGF-β1 was transferred to cells at 2 ng/ml, gently mixed and cultured at 37° C. for 24 hours. Quanti-Blue™ substrate was then transferred to a flat 96-well plate together with 40 μl of cell culture supernatants, mixed thoroughly and incubated at 37° ° C. for one hour. Secreted alkaline phosphatase (SEAP) levels in supernatants were measured in a spectrophotometer at 650 nm OD.

Results are shown in Table 14 and FIG. 10 and expressed as EC50. Reference antibody used was negative control RSV-G antibody. FAP×TGF-βRII bispecific antibodies indicated with SEQ ID NO: 15×SEQ ID NO: 31 and SEQ ID NO: 11×SEQ ID NO: 23 inhibited TGF-βRII signaling in HEK-Blue-TGF-βRII reporter cells, when these cells were incubated together with FAP-expressing MRC5 cells. FAP×TGF-βRII bispecific antibodies inhibited TGF-βRII signaling more than the control RSV-G×TGF-βRII antibodies comprising a heavy chain having an amino acid sequence as set forth in SEQ ID NO: 79 and a heavy chain having an amino acid sequence as set forth in SEQ ID NO: 31 or 23. The results indicate that FAP×TGF-βRII bispecific antibodies bound to HEK-Blue cells via the TGF-βRII binding domain and MRC5 cells via the FAP binding domain, to inhibit TGF-βRII mediated signaling in the HEK-Blue cells.

TABLE 14
TGF-βRII inhibition in trans by FAP ×
TGF-βRII bispecific antibodies.

| Antibody | EC50 HEKBlue/MRC5 |
|---|---|
| SEQ ID NO: 19 × SEQ ID NO: 27 | 4.948 |
| SEQ ID NO: 15 × SEQ ID NO: 31 | 0.1936 |
| SEQ ID NO: 11 × SEQ ID NO: 23 | 0.8439 |
| SEQ ID NO: 79 × SEQ ID NO: 27 | 3.393 |
| SEQ ID NO: 79 × SEQ ID NO: 31 | 14.74 |
| SEQ ID NO: 79 × SEQ ID NO: 23 | 0.9314 |
| Negative control RSV-G antibody | 1.36E−12 |

### Example 16: moFAP×huTGF-βRII Antibody Mediated Anti-Tumor Efficacy as Single Agent and in Combination with a PD-1 Inhibitor

Bispecific antibodies were characterized in vivo in a transgenic mouse model harboring human TGF-βRII expressing immune cells and mouse FAP (moFAP) expressing tumors, to determine their ability to inhibit TGF-βRII signaling on non-fibroblast cells in a trans-mode of activity. For this purpose, a CD34 humanized NSG mice was inoculated with MDA-MB-231 tumor cells and dosed with bispecific or reference antibodies.

Bispecific antibodies tested for trans-mode of activity (moFAP×TGF-βRII) were generated in Fc-silenced format, according to methods known in the art and comprise a mouse-FAP binding domain (moFAP) and a light chain variable region having an amino acid sequence as set forth in SEQ ID NO: 52, and a TGF-βRII binding domain, comprising a heavy chain variable region having an amino acid sequence as set forth in SEQ ID NO: 31 or SEQ ID NO: 23 and a light chain variable region having an amino acid sequence as set forth in SEQ ID NO: 52. The bispecific antibodies are referred to as moFAP×SEQ ID NO: 31 and moFAP×SEQ ID NO: 23. Reference antibodies used were negative control RSV-G antibody and analog reference TGF-βRII antibody TGF1.

Female CD34⁺ humanized mice (JAX) were inoculated with approximately 3 million MDA-MB-231 cells with 50% matrigel. Seven days after tumor implantation, mice were randomized by tumor volume and stem cell donor and dosed i.p. with the indicated agents twice a week. Mice received 7 total doses of antibodies.

TGI (tumor growth inhibition) was calculated from mean tumor volumes at day 24 using the formula (1−(VT/VC))×100, where VT is the tumor volume of the treatment group on the last day of treatment and VC is the tumor volume of the control group on the last day of treatment. Statistical analysis was performed using Two-Way ANOVA with Dunnett's Multiple Comparisons test.

In addition to their ability to inhibit TGF-βRII signaling on T cells, moFAP×TGF-βRII bispecific antibodies were also characterized for their ability to inhibit the PD-1/PD-L1 axis on T-cells. This was achieved by combining moFAP×TGF-βRII bispecific antibodies with pembrolizumab, an anti-PD-1 antibody. Treatment groups and details on dosing are provided in Table 15.

TABLE 15
Treatment groups for evaluation of TGF-βRII bispecific antibodies mediated
trans-mode of activity in vivo in humanized mice bearing MDA-MB-231 tumors.

| | No. of | | | | Dose | Total |
|---|---|---|---|---|---|---|
| Group | Animals | Antibody | mg/Kg | ug/mouse | schedule | Doses |
| 1 | 8 | Negative control RSV-G antibody | 10 | 250 | Q5D | 4 |
| 2 | 9 | Analog reference TGF1 antibody | 10 | 250 | Q5D | 4 |
| 3 | 9 | moFAP × SEQ ID NO: 23 | 1 | 25 | Q5D | 4 |
| 4 | 9 | moFAP × SEQ ID NO: 31 | 1 | 25 | Q5D | 4 |
| 5 | 9 | moFAP × SEQ ID NO: 23 | 10 | 250 | Q5D | 4 |
| 6 | 9 | moFAP × SEQ ID NO: 31 | 10 | 250 | Q5D | 4 |
| 7 | 9 | Pembrolizumab | 10 | 250 | Q5D | 4 |
| 8 | 9 | Analog reference TGF1 | 5 + 5 | 125 + 125 | Q5D | 4 |
| | | antibody + Pembrolizumab | | | | |
| 9 | 9 | moFAP × SEQ ID NO: 23 + | 5 + 5 | 125 + 125 | Q5D | 4 |
| | | Pembrolizumab | | | | |
| 10 | 9 | moFAP × SEQ ID NO: 31 + | 5 + 5 | 125 + 125 | Q5D | 4 |
| | | Pembrolizumab | | | | |

Results are shown in FIG. 8. When administered as a single agent, moFAP×TGF-βRII bispecific antibodies in treatment groups 3, 5 and 6 induced an anti-tumor response which was comparable to the anti-tumor response elicited by analog reference TGF1 antibody (Group 2). All groups, except Group 4, were found to result in significant tumor volume reduction when compared to negative control RSV-G antibody (p<0.05) for single agent treatment.

Results for the combination treatment are shown in FIG. 9. When tested in combination with pembrolizumab, moFAP×TGF-βRII bispecific antibodies resulted in tumor volume reduction, which was comparable to the tumor volume reduction achieved by analog reference TGF1 antibody and pembrolizumab used alone (Group 2 and 7, respectively) and in combination (Group 8).

It was noted that the bispecific antibodies which are monovalent for binding to TGF-βRII elicit a similar anti-tumor response as the bivalent monospecific analog reference antibody TGF1, at the same dosage level, in both single treatment and combination treatment experiments.

### Example 17: Macrophage Effector Function Assay

FAP×TGF-βRII bispecific antibodies were tested for their ability to mediate macrophage effector function in an antibody-dependent cellular phagocytosis (ADCP) assay. Bispecific antibodies indicated with SEQ ID NO: 15×SEQ ID NO: 35, in both Fc-silenced and Fc-enhanced format, were tested for ADCP activity using A549-FAP⁺ cells, or lung CAFs as target cells and M0/M2c macrophages as effector cells. An isotype IgG1 control antibody was the negative control for the assay. A549-FAP⁺ cells and lung CAFs were validated to express both TGF-βRII and FAP proteins (data not shown).

Macrophage differentiation protocol: Human peripheral blood monocytes were differentiated into M0/M2c macrophages as follows: Monocytes without CD16 depletion (CD14+CD16+) were isolated from human peripheral blood using EasySep Human Monocyte isolation Kit (Stemcell technologies, cat. no. 19058) as per manufacturer's instructions and resuspended in XVIVO10 media (Lonza, cat. no. BEBP02-055Q) containing 10% FBS. Cells were counted on cellometer and plated in two 150 mm petri dishes at 15×10⁶/30 mL with M-CSF (25 ng/mL, (R&D systems cat. no. 216-MC-025/CF)) for 3 days (72 hours) at 37° C., 5% CO₂ incubator. On day 4, after half media change, IL-10 (10 ng/ml; R&D systems cat. no. 217-IL/CF) was added to one petri dish for 48 hours to differentiate into M2c macrophages and same volume of media with no IL-10 was added to another petri dish for M0 macrophages. After washing of the petri dishes with sterile PBS, macrophages were harvested by incubation with Accutase™ (20 mL; Millipore cat. no. SCR005) in 37° ° C. incubator for 10 min. Cells were counted and used for staining with macrophage marker antibodies and the ADCP assay.

Differentiated macrophages were stained with live/dead Fix Aqua dye (100 uL from 1:1000 in 1×DPBS; Invitrogen cat. no. L34957) for 15 mins at room temperature. Cells were washed with BSA stain buffer (300 uL; BD cat. no. 554657) by centrifuging at 500×g for 5 mins at room temperature and the supernatant discarded. 100 uL Fc block (1:20 in staining buffer) was added and incubated for 10 min at room temperature, followed by another washing step with BSA stain buffer. 50 uL antibody mix containing CD11b BC421, CD163 APC, CD206 BUV737, CD80 BV650, HLA-DR PerCP Cy5.5, PD1 PE, TGFBR2 was added to sample tubes and incubated for 30 min on ice. Flowcytometry minus one (FMO) controls were included for CD163, CD206, CD80, HLADR, PD1, TGF-βRII for interpretation of flow cytometry data. Cells were washed with BSA stain buffer (300 μL) and centrifuged at 500×g for 5 mins at room temperature and resuspended in 300 μL BSA stain buffer. Acquisition was performed on LSR Fortessa X-20.

M2c macrophages were validated to specifically express M1/M2 markers. M2c macrophages also expressed TGF-βRII but not FAP (data not shown).

The ability of FAPxTGF-βRII bispecific antibodies to mediate macrophage effector function on A549-FAP⁺ cells was tested by flow cytometry and lung CAFs by IncuCyte Live cell imaging.

ADCP assay: Target cells (A549-FAP⁺ cells and lung CAFs) were trypsinized, washed, resuspended at 1×10⁶ cells/mL and labeled with 2 μM CFSE (Invitrogen cat. no. C34554) as per manufacturer's instructions. 50 uL of labeled target cells were added at the indicated concentration to 96 well u-bottom polypropylene plates (for 50,000 cells target cells in 50 uL, cell concentration required is 1×10⁶/mL). 100 uL bispecific antibodies indicated with SEQ ID NO: 15×SEQ ID NO: 35, in both Fc-silenced and Fc-enhanced format, and isotype control antibody were added at concentrations ranging from 10 ug/mL to 0.001 ug/mL (10-fold dilutions) for 30 min at room temperature. No antibody control (0 ug/mL) was also included. Harvested macrophages as described earlier were resuspended at the indicated concentrations at 50 uL/well to 96 well u-bottom polypropylene plate (Effector:Target cell ratio: 1:1 for all target cell types). A549-FAP⁺ cells and Lung CAFs were tested at both Effector:Target cell ratios of 1:1 and 3:1.

Flow cytometry: The cell/antibody suspension was mixed and centrifuged at 800 rpm for 3 min to concentrate cells at the bottom. The plate was incubated for 24 hours at 37° C., 5% CO₂ and washed by centrifuging at 500×g for 5 min. Cell pellets were stained with live dead aqua dye (100 uL from 1:1000 in 1×DPBS) and incubated for 15 min at room temperature. 100 uL of BSA stain buffer was added and cells centrifuged at 500×g, 5 min at room temperature. The supernatant was discarded and 100 uL Fc block (1:20 in staining buffer) was added and incubated for 10 min at room temperature. 300 uL of BSA stain buffer was added again, cells centrifuged at 500×g, 5 min at room temperature and the supernatant discarded. Cells were stained with 50 uL of anti-CD11b APC-Cy7 (2 μL antibody+48 μL BSA Stain buffer; BD Biosciences cat. no. 557754) for 30 mins on ice. Cells were washed again with 300 μL of BSA stain buffer, centrifuged at 500×g, 5 min at room temperature and supernatant discarded. Acquisition was performed on LSR Fortessa X-20.

Live cell imaging: Macrophages were labelled with 3 μM Cell Tracker Red CMPTX dye (Invitrogen cat. no. C34552) as per the manufacturer's recommendations and the mixture of effector and target cells (based on the indicated ratios) was added to 96 well polystyrene flat bottom plates. Cell/antibody suspension was mixed, and the plate centrifuged at 800 rpm for 3 min to concentrate cells at the bottom. The plate was then placed on IncuCyte Live cell imaging incubator (Leica microsystems) for data acquisition.

Results are shown in FIG. 11. The FAPxTGF-βRII Fc-enhanced antibody resulted in a higher dose-dependent increase in phagocytosis of A549-FAP⁺ cells (FIG. 11A) for both effector:target cell ratios of 1:1 and 3:1 than the FAPxTGF-βRII Fc-silenced antibody, as measured by flow cytometry. Killing of lung CAFs by M2c macrophages was also observed with the Fc enhanced FAPxTGF-βRII bispecific antibody (FIG. 11B) but not with the Fc silenced FAPxTGF-βRII bispecific antibody, as measured by IncuCyte Live cell imaging. The negative control IgG1 isotype antibody did not mediate ADCP killing of A549-FAP⁺ or lung CAFs.

SEQUENCES

SEQ ID NO: 1 Heavy chain reference anti-TGF-βRII antibody TGF1 analog
QLQVQESGPGLVKPSETLSLTCTVSGGSISNSYFSWGWIRQPPGKGLEWIGSFYYGEKTY
YNPSLKSRATISIDTSKSQFSLKLSSVTAADTAVYYCPRGPTMIRGVIDSWGQGTLVTVSS
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKRVEPKSCDKTHTCPPCPAPELLGG
PSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSR
EEMTKNQVSLTCLVKGFYPSDIAVEWESNGQPENNYKTTPPVLDSDGSFFLYSKLTVDK
SRWQQGNVFSCSVMHEALHNHYTQKSLSLSPGK
SEQ ID NO: 2 Light chain reference anti-TGF-βRII antibody TGF1 analog
EIVLTQSPATLSLSPGERATLSCRASQSVRSYLAWYQQKPGQAPRLLIYDASNRATGIPA
RFSGSGSGTDFTLTISSLEPEDFAVYYCQQRSNWPPTFGQGTKVEIKRTVAAPSVFIFPPS
DEQLKSGTASVVCLLNNFYPREAKVQWKVDNALQSGNSQESVTEQDSKDSTYSLSSTL
TLSKADYEKHKVYACEVTHQGLSSPVTKSFNRGEC
SEQ ID NO: 3 Heavy chain negative control RSV IgG1 antibody
EVQLVESGGGVVQPGRSLRLSCAASGFTFSNYGMHWVRQAPGKGLEWVAVISYDGST
KYSADSLKGRFTISRDNSKNTLYLQMNSLRADDTAVYYCAKEGWSFDSSGYRSWFDS
WGQGTLVTVSSASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDKRVEPKSCDKTHT
CPPCPAPELLGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPG
SEQ ID NO: 4 Light chain
DIQMTQSPSSLSASVGDRVTITCRASQSISSYLNWYQQKPGKAPKLLIYAASSLQSGVPSR
FSGSGSGTDFTLTISSLQPEDFATYYCQQSYSTPPTFGQGTKVEIKRTVAAPSVFIFPPSDE
QLKSGTASVVCLLNNFYPREAKVQWKVDNALQSGNSQESVTEQDSKDSTYSLSSTLTLS
KADYEKHKVYACEVTHQGLSSPVTKSFNRGEC
SEQ ID NO: 5 Heavy chain negative control RSV IgGI antibody
EVQLVESGGGVVQPGRSLRLSCAASGFTFSNYGMHWVRQAPGKGLEWVAVISYDGST
KYSADSLKGRFTISRDNSKNTLYLQMNSLRADDTAVYYCAKEGWSFDSSGYRSWFDS
WGQGTLVTVSSASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDKRVEPKSCDKTHT
CPPCPAPELGRGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPG
SEQ ID NO: 6 huFAP
MKTWVKIVFGVATSAVLALLVMCIVLRPSRVHNSEENTMRALTLKDILNGTFSYKTFF
PNWISGQEYLHQSADNNIVLYNIETGQSYTILSNRTMKSVNASNYGLSPDRQFVYLESD
YSKLWRYSYTATYYIYDLSNGEFVRGNELPRPIQYLCWSPVGSKLAYVYQNNIYLKQRP
GDPPFQITFNGRENKIFNGIPDWVYEEEMLATKYALWWSPNGKFLAYAEFNDTDIPVIA
YSYYGDEQYPRTINIPYPKAGAKNPVVRIFIIDTTYPAYVGPQEVPVPAMIASSDYYFSW
LTWVTDERVCLQWLKRVQNVSVLSICDFREDWQTWDCPKTQEHIEESRTGWAGGFFVS
TPVFSYDAISYYKIFSDKDGYKHIHYIKDTVENAIQITSGKWEAINIFRVTQDSLFYSSNEF
EEYPGRRNIYRISIGSYPPSKKCVTCHLRKERCQYYTASFSDYAKYYALVCYGPGIPISTL
HDGRTDQEIKILEENKELENALKNIQLPKEEIKKLEVDEITLWYKMILPPQFDRSKKYPLL
IQVYGGPCSQSVRSVFAVNWISYLASKEGMVIALVDGRGTAFQGDKLLYAVYRKLGVY
EVEDQITAVRKFIEMGFIDEKRIAIWGWSYGGYVSSLALASGTGLFKCGIAVAPVSSWEY
YASVYTERFMGLPTKDDNLEHYKNSTVMARAEYFRNVDYLLIHGTADDNVHFQNSAQI
AKALVNAQVDFQAMWYSDQNHGLSGLSTNHLYTHMTHFLKQCFSLSD
SEQ ID NO: 7 Heavy chain analog reference FAP antibody sibrotuzumab
QVQLVQSGAEVKKPGASVKVSCKTSRYTFTEYTIHWVRQAPGQRLEWIGGINPNNGIPN
YNQKFKGRVTITVDTSASTAYMELSSLRSEDTAVYYCARRRIAYGYDEGHAMDYWGQ
GTLVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKKVEPKSCDKTHTCPPC
PAPELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 8 Light chain analog reference FAP antibody sibrotuzumab
DIVMTQSPDSLAVSLGERATINCKSSQSLLYSRNQKNYLAWYQQKPGQPPKLLIFWAST
RESGVPDRFSGSGFGTDFTLTISSLQAEDVAVYYCQQYFSYPLTFGQGTKVEIKRTVAAP
SVFIFPPSDEQLKSGTASVVCLLNNFYPREAKVQWKVDNALQSGNSQESVTEQDSKDST
YSLSSTLTLSKADYEKHKVYACEVTHQGLSSPVTKSFNRGEC
SEQ ID NO: 9 cyFAP
MKTWVKIVFGVATSAVLALLVMCIVLRPPRVHNSEENTMRALTLKDILNGTFSYKTFF
PNWISGQEYLHQSADNNIVLYNIETGQSYTILSNRTMKSVNASNYGLSPDRQFVYLESD
YSKLWRYSYTATYYIYDLSNGEFVRGNELPRPIQYLCWSPVGSKLAYVYQNNIYLKQRP
GDPPFQITFNGRENKIFNGIPDWVYEEEMLATKYALWWSPNGKFLAYAEFNDTDIPVIA
YSYYGDEQYPRTINIPYPKAGAKNPFVRIFIIDTTYPAYVGPQEVPVPAMIASSDYYFSWL
TWVTDERVCLQWLKRVQNVSVLSICDFREDWQTWDCPKTQEHIEESRTGWAGGFFVST
PVFSYDAISYYKIFSDKDGYKHIHYIKDTVENAIQITSGKWEAINIFRVTQDSLFYSSNEFE
DYPGRRNIYRISIGSYPPSKKCVTCHLRKERCQYYTASFSDYAKYYALVCYGPGIPISTLH
DGRTDQEIKILEENKELENALKNIQLPKEEIKKLEVDEITLWYKMILPPQFDRSKKYPLLI
QVYGGPCSQSVRSVFAVNWISYLASKEGMVIALVDGRGTAFQGDKLLYAVYRKLGVY
EVEDQITAVRKFIEMGFIDEKRIAIWGWSYGGYVSSLALASGTGLFKCGIAVAPVSSWEY
YASVYTERFMGLPTKDDNLEHYKNSTVMARAEYFRNVDYLLIHGTADDNVHFQNSAQI
AKALVNAQVDFQAMWYSDQNHGLSGLSTNHLYTHMTHFLKQCFSLSD
SEQ ID NO: 10 huCD26
MKTPWKVLLGLLGAAALVTIITVPVVLLNKGTDDATADSRKTYTLTDYLKNTYRLKLY
SLRWISDHEYLYKQENNILVFNAEYGNSSVFLENSTFDEFGHSINDYSISPDGQFILLEYN
YVKQWRHSYTASYDIYDLNKRQLITEERIPNNTQWVTWSPVGHKLAYVWNNDIYVKIE
PNLPSYRITWTGKEDIIYNGITDWVYEEEVFSAYSALWWSPNGTFLAYAQFNDTEVPLIE
YSFYSDESLQYPKTVRVPYPKAGAVNPTVKFFVVNTDSLSSVTNATSIQITAPASMLIGD
HYLCDVTWATQERISLQWLRRIQNYSVMDICDYDESSGRWNCLVARQHIEMSTTGWV
GRFRPSEPHFTLDGNSFYKIISNEEGYRHICYFQIDKKDCTFITKGTWEVIGIEALTSDYLY
YISNEYKGMPGGRNLYKIQLSDYTKVTCLSCELNPERCQYYSVSFSKEAKYYQLRCSGP
GLPLYTLHSSVNDKGLRVLEDNSALDKMLQNVQMPSKKLDFIILNETKFWYQMILPPHF
DKSKKYPLLLDVYAGPCSQKADTVFRLNWATYLASTENIIVASFDGRGSGYQGDKIMH
AINRRLGTFEVEDQIEAARQFSKMGFVDNKRIAIWGWSYGGYVTSMVLGSGSGVFKCGI
AVAPVSRWEYYDSVYTERYMGLPTPEDNLDHYRNSTVMSRAENFKQVEYLLIHGTAD
DNVHFQQSAQISKALVDVGVDFQAMWYTDEDHGIASSTAHQHIYTHMSHFIKQCFSLP
SEQ ID NO: 11 Heavy Chain Variable region
EVQLVESGGGLVQPGGSLRLSCAASGFTFSSYAMIWVRQAPGKGLEWVSAISGSGGYTF
NADSVKGRFTMSRDNSKNTLYLQMNSLRAEDTAVYYCAKRDGGYEGGAFDIWGQGTL
VTVSS
SEQ ID NO: 12 Heavy Chain CDR1
SYAMI
SEQ ID NO: 13 Heavy Chain CDR2
AISGSGGYTFNADSVKG
SEQ ID NO: 14 Heavy Chain CDR3
RDGGYEGGAFDI
SEQ ID NO: 15 Heavy Chain Variable region
QLQLQESGPGLVKPSETLSLTCTVSGGSISSSSYFWAWIRQPPGKGLEFIGNIYYSGSTYY
NPSLKSRVTISVDTSKNQFSLKLSSVTAADTAVYYCARPSYYGSGNYYFAFWGQGTLVT
VSS
SEQ ID NO: 16 Heavy Chain CDR1
SSSYFWA
SEQ ID NO: 17 Heavy Chain CDR2
NIYYSGSTYYNPSLKS
SEQ ID NO: 18 Heavy Chain CDR3
PSYYGSGNYYFAF
SEQ ID NO: 19 Heavy Chain Variable region
EVQLVESGGGLVKPGGSLRLSCAASGFPFNYAWMSWVRQAPGKGLEWVGRIKPKTSG
GATDYAAPVKDRFTISRDDSRNTLYLQMNSLKTEDTAVYYCSAREDIWNFFFDLWGQG
TLVTVSS
SEQ ID NO: 20 Heavy Chain CDR1
YAWMS
SEQ ID NO: 21 Heavy Chain CDR2
RIKPKTSGGATDYAAPVKD
SEQ ID NO: 22 Heavy Chain CDR3
REDIWNFFFDL
SEQ ID NO: 23 Heavy Chain Variable region
QVQLVESGGGLVQPGGSLRLSCAVSGFTFRRYAMSWVRQAPGKGLEWVSAISASGDRT
HNTDSVKGRFSISRDNSKNTLYLQMNSLRAEDTAVYFCAKGIAASGKNYFDPWGQGTL
VTVSS
SEQ ID NO: 24 Heavy Chain CDR1
RYAMS
SEQ ID NO: 25 Heavy Chain CDR2
AISASGDRTHNTDSVKG
SEQ ID NO: 26 Heavy Chain CDR3
GIAASGKNYFDP
SEQ ID NO: 27 Heavy Chain Variable region
QVQLVESGGGLVQPGGSLRLSCAVSGFTFSRYAMSWVRQAPGKGLEWVSAISASGDRT
KNTDSVKGRFSISRDNSKNTLYLQMNSLRAEDTAVYFCAKGTAAAGKNYFDPWGQGT
LVTVSS
SEQ ID NO: 28 Light chain V-region
EIVLTQSPGTLSLSPGERATLSCRASQSVSSSYLAWYQQKPGQAPRLLIYGASSRATGIPD
RFSGSGSGTDFTLTISRLEPEDFAVYYCQQYGSSP
SEQ ID NO: 29 Heavy Chain CDR2
AISASGDRTKNTDSVKG
SEQ ID NO: 30 Heavy Chain CDR3
GTAAAGKNYFDP
SEQ ID NO: 31 Heavy Chain Variable region
QVQLVESGGGLVQPGGSLRLSCAVSGFTFERYAMSWVRQAPGKGLEWVSAISASGDRT
QNTDSVKGRFSISRDNSKNTLYLQMNSLRAEDTAVYFCAKGTAASGRNYFDPWGQGTL
VTVSS
SEQ ID NO: 32 Light chain variable region
SYVLTQPPSVSVAPGETARITCGGDNIGRKSVYWYQQKSGQAPVLVIYYDSDRPSGIPER
FSGSNSGNTATLTISRVEAGDEADYYCQVWDGSSDHWVFGGGTKLTVL
SEQ ID NO: 33 Heavy Chain CDR2
AISASGDRTQNTDSVKG
SEQ ID NO: 34 Heavy Chain CDR3
GTAASGRNYFDP
SEQ ID NO: 35 Heavy Chain Variable region
QVQLVESGGGLVQPGGSLRLSCAVSGFTFERYAMSWVRQAPGKGLEWVSAISASGDRT
QYTDSVKGRFSISRDNSKNTLYLQMNSLRAEDTAVYFCAKGTAASGRNYFDPWGQGTL
VTVSS
SEQ ID NO: 36 LCDR1 indicated according to IMGT
NIGRKS
SEQ ID NO: 37 Heavy Chain CDR2
AISASGDRTQYTDSVKG
SEQ ID NO: 38 LCDR2 indicated according to IMGT
YDS
SEQ ID NO: 39 CH1 region
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKRV
SEQ ID NO: 40 Hinge
EPKSCDKTHTCPPCP
SEQ ID NO: 41 CH2 region un-modified effector function
APELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAK
SEQ ID NO: 42 CH2-DM region
APELGRGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAK
SEQ ID NO: 43 CH3 region
GQPREPQVYTLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPG
SEQ ID NO: 44 CH3-DE region
GQPREPQVYTDPPSREEMTKNQVSLTCEVKGFYPSDIAVEWESNGQPENNYKTTPPVLD
SDGSFFLYSKLTVDKSRWQQGNVFSCSVMHEALHNHYTQKSLSLSPG
SEQ ID NO: 45 CH3-KK region
GQPREPQVYTKPPSREEMTKNQVSLKCLVKGFYPSDIAVEWESNGQPENNYKTTPPVLD
SDGSFFLYSKLTVDKSRWQQGNVFSCSVMHEALHNHYTQKSLSLSPG
SEQ ID NO: 46 human TGF-βRII isoform A
MGRGLLRGLWPLHIVLWTRIASTIPPHVQKSVNNDMIVTDNNGAVKFPQLCKFCDVRFS
TCDNQKSCMSNCSITSICEKPQEVCVAVWRKNDENITLETVCHDPKLPYHDFILEDAASP
KCIMKEKKKPGETFFMCSCSSDECNDNIIFSEEYNTSNPDLLL VIFQVTGISLLPPLGVAIS
VIIIFYCYRVNRQQKLSSTWETGKTRKLMEFSEHCAIILEDDRSDISSTCANNINHNTELLP
IELDTLVGKGRFAEVYKAKLKQNTSEQFETVAVKIFPYEEYASWKTEKDIFSDINLKHEN
ILQFLTAEERKTELGKQYWLITAFHAKGNLQEYLTRHVISWEDLRKLGSSLARGIAHLHS
DHTPCGRPKMPIVHRDLKSSNILVKNDLTCCLCDFGLSLRLDPTLSVDDLANSGQVGTA
RYMAPEVLESRMNLENVESFKQTDVYSMALVLWEMTSRCNAVGEVKDYEPPFGSKVR
EHPCVESMKDNVLRDRGRPEIPSFWLNHQGIQMVCETLTECWDHDPEARLTAQCVAER
FSELEHLDRLSGRSCSEEKIPEDGSLNTTK
SEQ ID NO: 47 extracellular domain of human TGF-βRII isoform A
TIPPHVQKSVNNDMIVTDNNGAVKFPQLCKFCDVRFSTCDNQKSCMSNCSITSICEKPQE
VCVAVWRKNDENITLETVCHDPKLPYHDFILEDAASPKCIMKEKKKPGETFFMCSCSSD
ECNDNIIFSEEYNTSNPDLLLVIFQ
SEQ ID NO: 48 human TGF-βRII isoform B
MGRGLLRGLWPLHIVLWTRIASTIPPHVQKSDVEMEAQKDEIICPSCNRTAHPLRHINND
MIVTDNNGAVKFPQLCKFCDVRFSTCDNQKSCMSNCSITSICEKPQEVCVAVWRKNDE
NITLETVCHDPKLPYHDFILEDAASPKCIMKEKKKPGETFFMCSCSSDECNDNIIFSEEYN
TSNPDLLLVIFQVTGISLLPPLGVAISVIIIFYCYRVNRQQKLSSTWETGKTRKLMEFSEHC
AIILEDDRSDISSTCANNINHNTELLPIELDTLVGKGRFAEVYKAKLKQNTSEQFETVAVK
IFPYEEYASWKTEKDIFSDINLKHENILQFLTAEERKTELGKQYWLITAFHAKGNLQEYL
TRHVISWEDLRKLGSSLARGIAHLHSDHTPCGRPKMPIVHRDLKSSNIL VKNDLTCCLCD
FGLSLRLDPTLSVDDLANSGQVGTARYMAPEVLESRMNLENVESFKQTDVYSMALVL
WEMTSRCNAVGEVKDYEPPFGSKVREHPCVESMKDNVLRDRGRPEIPSFWLNHQGIQM
VCETLTECWDHDPEARLTAQCVAERFSELEHLDRLSGRSCSEEKIPEDGSLNTTK
SEQ ID NO: 49 extracellular domain of isoform B of human TGF-βRII
TIPPHVQKSDVEMEAQKDEIICPSCNRTAHPLRHINNDMIVTDNNGAVKFPQLCKFCDVR
FSTCDNQKSCMSNCSITSICEKPQEVCVAVWRKNDENITLETVCHDPKLPYHDFILEDAA
SPKCIMKEKKKPGETFFMCSCSSDECNDNIIFSEEYNTSNPDLLL VIFQ
SEQ ID NO: 50 huPD-1
MQIPQAPWPVVWAVLQLGWRPGWFLDSPDRPWNPPTFSPALLVVTEGDNATFTCSFSN
TSESFVLNWYRMSPSNQTDKLAAFPEDRSQPGQDCRFRVTQLPNGRDFHMSVVRARRN
DSGTYLCGAISLAPKAQIKESLRAELRVTERRAEVPTAHPSPSPRPAGQFQTLVVGVVGG
LLGSLVLLVWVLAVICSRAARGTIGARRTGQPLKEDPSAVPVFSVDYGELDFQWREKTP
EPPVPCVPEQTEYATIVFPSGMGTSSPARRGSADGPRSAQPLRPEDGHCSWPL
SEQ ID NO: 51 Light chain constant region
RTVAAPSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC
SEQ ID NO: 52 Light chain variable region
DIQMTQSPSSLSASVGDRVTITCRASQSISSYLNWYQQKPGKAPKLLIYAASSLQSGVPSR
FSGSGSGTDFTLTISSLQPEDFATYYCQQSYSTPPTFGQGTKVEIK
SEQ ID NO: 53 LCDR1 indicated according to IMGT
QSISSY
SEQ ID NO: 54 LCDR2 indicated according to IMGT
AAS
SEQ ID NO: 55 LCDR3 indicated according to IMGT
QQSYSTPPT
SEQ ID NO: 56 Light chain variable region
DIQMTQSPSSLSASVGDRVTITCRASQSISSYLNWYQQKPGKAPKLLIYAASSLQSGVPSR
FSGSGSGTDFTLTISSLQPEDFATYYCQQSYSTPPITFGQGTRLEIK
SEQ ID NO: 57 LCDR3 indicated according to IMGT
QVWDGSSDHWV
SEQ ID NO: 58 Light chain V-region
SYVLTQPPSVSVAPGETARITCGGDNIGRKSVYWYQQKSGQAPVLVIYYDSDRPSGIPER
FSGSNSGNTATLTISRVEAGDEADYYCQVWDGSSDH
SEQ ID NO: 59 LCDR3 indicated according to IMGT
QQSYSTPPIT
SEQ ID NO: 60 Light chain V-region
DIQMTQSPSSLSASVGDRVTITCRASQSISSYLNWYQQKPGKAPKLLIYAASSLQSGVPSR
FSGSGSGTDFTLTISSLQPEDFATYYCQQSYSTP
SEQ ID NO: 61 Light chain variable region
EIVMTQSPATLSVSPGERATLSCRASQSVSSNLAWYQQKPGQAPRLLIYGASTRATGIPA
RFSGSGSGTEFTLTISSLQSEDFAVYYCQQYNNWPWTFGQGTKVEIK
SEQ ID NO: 62 LCDR1 indicated according to IMGT
QSVSSN
SEQ ID NO: 63 LCDR2 indicated according to IMGT
GAS
SEQ ID NO: 64 LCDR3 indicated according to IMGT
QQYNNWPWT
SEQ ID NO: 65 Light chain V-region
EIVMTQSPATLSVSPGERATLSCRASQSVSSNLAWYQQKPGQAPRLLIYGASTRATGIPA
RFSGSGSGTEFTLTISSLQSEDFAVYYCQQYNNWP
SEQ ID NO: 66 Light chain variable region
EIVLTQSPGTLSLSPGERATLSCRASQSVSSSYLAWYQQKPGQAPRLLIYGASSRATGIPD
RFSGSGSGTDFTLTISRLEPEDFAVYYCQQYGSSPWTFGQGTKVEIK
SEQ ID NO: 67 LCDR1 indicated according to IMGT
QSVSSSY
SEQ ID NO: 68 LCDR3 indicated according to IMGT
QQYGSSPWT
SEQ ID NO: 69-Heavy chain variable region
EVQLVETGGGLIQPGGSLRLSCAASGFTVSSNYMSWVRQAPGKGLEWVSVIYSGGSTY
YADSVKGRFTISRDNSKNTLYLQMNSLRAEDTAVYYCARGPTDAYPYLDYLWGQGTL
VTVSS
SEQ ID NO: 70-Heavy chain CDR1
SNYMS
SEQ ID NO: 71-Heavy chain CDR 2
VIYSGGSTYYADSVKG
SEQ ID NO: 72-Heavy chain CDR 3
GPTDAYPYLDYL
SEQ ID NO: 73-Mouse FAP
MKTWLKTVFGVTTLAALALVVICIVLRPSRVYKPEGNTKRALTLKDILNGTFSYKTYF
PNWISEQEYLHQSEDDNIVFYNIETRESYIILSNSTMKSVNATDYGLSPDRQFVYLESDYS
KLWRYSYTATYYIYDLQNGEFVRGYELPRPIQYLCWSPVGSKLAYVYQNNIYLKQRPG
DPPFQITYTGRENRIFNGIPDWVYEEEMLATKYALWWSPDGKFLAYVEFNDSDIPIIAYS
YYGDGQYPRTINIPYPKAGAKNPVVRVFIVDTTYPHHVGPMEVPVPEMIASSDYYFSWL
TWVSSERVCLQWLKRVQNVSVLSICDFREDWHAWECPKNQEHVEESRTGWAGGFFVS
TPAFSQDATSYYKIFSDKDGYKHIHYIKDTVENAIQITSGKWEAIYIFRVTQDSLFYSSNE
FEGYPGRRNIYRISIGNSPPSKKCVTCHLRKERCQYYTASFSYKAKYYALVCYGPGLPIST
LHDGRTDQEIQVLEENKELENSLRNIQLPKVEIKKLKDGGLTFWYKMILPPQFDRSKKYP
LLIQVYGGPCSQSVKSVFAVNWITYLASKEGIVIALVDGRGTAFQGDKFLHAVYRKLGV
YEVEDQLTAVRKFIEMGFIDEERIAIWGWSYGGYVSSLALASGTGLFKCGIAVAPVSSW
EYYASIYSERFMGLPTKDDNLEHYKNSTVMARAEYFRNVDYLLIHGTADDNVHFQNSA
QIAKALVNAQVDFQAMWYSDQNHGISSGRSQNHLYTHMTHFLKQCFSLSD
SEQ ID NO: 74-Heavy chain ESC11 analog
QVQLQESGPGLVKPSETLSLTCTVSGGSISSNNYYWGWIRQTPGKGLEWIGSIYYSGSTN
YNPSLKSRVTISVDTSKNQFSLKLSSVTAADTAVYYCARGARWQARPATRIDGVAFDIW
GQGTMVTVSSASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKRVEPKSCDKTHTC
PPCPAPELLGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQP
REPQVYTLPPSREEMTKNQVSLTCLVKGFYPSDIAVEWESNGQPENNYKTTPPVLDSDG
SFFLYSKLTVDKSRWQQGNVFSCSVMHEALHNHYTQKSLSLSPGK
SEQ ID NO: 75-Light chain ESC11 analog
EIVLTQSPGTLSLSPGERATLSCRASQTVTRNYLAWYQQKPGQAPRLLMYGASNRAAG
VPDRFSGSGSGTDFTLTISRLEPEDFA VYYCQQFGSPYTFGQGTVEIKRTVAAPSVFIFPPS
DEQLKSGTASVVCLLNNFYPREAKVQWKVDNALQSGNSQESVTEQDSKDSTYSLSSTL
TLSKADYEKHKVYACEVTHQGLSSPVTKSFNRGEC
SEQ ID NO: 76-Heavy chain IgGI hu/moFAP
EVQLVQSGAEVKKPGASVKVSCKASGYTFTSYGISWVRQAPGQGLEWMGWISAYNGN
TNYAQKLQGRVTMTTDTSTSTAYMELRSLRSDDTAVYYCARDWSRSGYYLPDYWGQ
GTLVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDKTHTCPPC
PAPELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 77-Light chain IgG1 hu/moFAP
DVVMTQSPLSLPVTLGQPASISCRSSQSLLHSNGYNYLDWYLQRPGQSPHLLIFLGSNRA
SGVPDRFSGSGSGTDFTLKISR VEAEDVGIYYCMQALQTPPTFGQGTKVEIKRTVAAPSV
FIFPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC
SEQ ID NO: 78-Heavy chain IgGI negative control TT
EVQLVETGAEVKKPGASVKVSCKASDYIFTKYDINWVRQAPGQGLEWMGWMSANTG
NTGYAQKFQGRVTMTRDTSINTAYMELSSLTSGDTAVYFCARSSLFKTETAPYYHFALD
VWGQGTTVTVSSASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVDKRVEPKSCDKTH
TCPPCPAPELLGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKAKG
QPREPQVYTLPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPG
SEQ ID NO: 79-Heavy chain variable region negative control RSV IgG1 antibody
EVQLVESGGGVVQPGRSLRLSCAASGFTFSNYGMHWVRQAPGKGLEWVAVISYDGST
KYSADSLKGRFTISRDNSKNTLYLQMNSLRADDTAVYYCAKEGWSFDSSGYRSWFDS
WGQGTLVTVSS

### CLAUSES

1. A bispecific binding moiety comprising a FAP binding domain and a TGF-βRII binding domain, wherein the TGF-βRII binding domain blocks TGF-βRII binding to TGF-βRII ligand.

2. The bispecific binding moiety according to clause 1, wherein the bispecific binding moiety blocks TGF-βRII mediated signaling in a cell expressing FAP and TGF-βRII.

3. The bispecific binding moiety according to any of the preceding clauses, wherein the potency of the bispecific binding moiety in blocking TGF-βRII mediated signaling is 2.0-500 fold higher than the potency of a reference anti-TGF-βRII antibody in a cell expressing FAP and TGF-βRII, wherein the reference anti-TGF-βRII antibody is a bivalent monospecific antibody comprising a heavy chain having an amino acid sequence as set forth in SEQ ID NO: 1 and a light chain having an amino acid sequence as set forth in SEQ ID NO: 2.

4. The bispecific binding moiety according to any of the preceding clauses, wherein the blocking of TGF-βRII mediated signaling is measured as reduction in pSMAD2 expression in a TGF-βRII signaling inhibition assay.

5. The bispecific binding moiety according to any of the preceding clauses, wherein the cell expressing FAP and TGF-βRII is a human fibroblast.

6. The bispecific binding moiety according to any of the preceding clauses, wherein the cell expressing FAP and TGF-βRII is a primary cancer associated fibroblast (CAF).

7. The bispecific binding moiety according to any of the preceding clauses, wherein the bispecific binding moiety has a higher potency in blocking TGF-βRII mediated signaling in a cell expressing FAP and TGF-βRII than in a cell expressing TGF-βRII and no, or undetectable levels of FAP.

8. The bispecific binding moiety according to any of the preceding clauses, wherein the cell expressing FAP and TGF-βRII is a A549-FAP⁺ cell and wherein the cell expressing TGF-βRII and no, or undetectable levels of FAP is a A549 parental cell.

9. The bispecific binding moiety according to any of the preceding clauses, wherein the potency in blocking TGF-βRII mediated signaling is measured as a reduction in pSMAD2 levels in a mixed culture pSMAD2 assay.

10. The bispecific binding moiety according to any of the preceding clauses, wherein the potency of the bispecific binding moiety in blocking TGF-βRII mediated signaling in a cell expressing FAP and TGF-βRII is between 100-20,000 fold higher than in a cell expressing TGF-βRII and no, or undetectable levels of FAP.

11. A bispecific binding moiety any of the preceding clauses, wherein the FAP binding domain binds to FAP expressed on a first cell and the TGF-βRII binding domain binds to TGF-βRII expressed on a second cell.

12. The bispecific binding moiety according to any of the preceding clauses, wherein upon binding of the FAP binding domain to FAP expressed on the first cell and binding of the TGF-βRII binding domain to TGF-βRII expressed on the second cell, the TGF-βRII binding domain blocks TGF-βRII mediated signaling in the second cell.

13. The bispecific binding moiety according to any of the preceding clauses, wherein the first cell is a fibroblast cell.

14. The bispecific binding moiety according to any of the preceding clauses, wherein the second cell is a non-fibroblast cell.

15. The bispecific binding moiety according to any of the preceding clauses, wherein the second cell is an immune effector cell or a tumor cell.

16. The bispecific binding moiety according to any of the preceding clauses, wherein the second cell is a T-cell.

17. The bispecific binding moiety according to any of the preceding clauses, wherein the blocking of TGF-βRII mediated signaling in the second cell is measured in a TGF-β reporter assay as described in Example 15.

18. The bispecific binding moiety according to any of the preceding clauses, wherein the first cell used in the TGF-β reporter assay is a FAP expressing MRC5 cell and the second cell is a TGF-βRII expressing HEK-Blue cell.

19. The bispecific binding moiety according to any of the preceding clauses, wherein the affinity of the FAP binding domain for human FAP is 25-50 fold higher than the affinity of TGF-βRII binding domain for human TGF-βRII.

20. The bispecific binding moiety according to any of the preceding clauses, wherein the affinity of the FAP binding domain is 0.1-0.2 nM and the affinity of the TGF-βRII binding domain is 3.8-5 nM.

21. The bispecific binding moiety according to any of the preceding clauses, wherein the affinity is determined as the equilibrium dissociation constant K_{D} as measured by SPR.

22. A bispecific binding moiety any of the preceding clauses, wherein the FAP binding domain comprises a heavy chain variable region comprising:
heavy chain CDR1 (HCDR1), heavy chain CDR2 (HCDR2), and heavy chain CDR3 (HCDR3), having an amino acid sequence as set forth in SEQ ID NO: 12, SEQ ID NO: 13, and SEQ ID NO: 14, respectively,
heavy chain CDR1 (HCDR1), heavy chain CDR2 (HCDR2), and heavy chain CDR3 (HCDR3), having an amino acid sequence as set forth in SEQ ID NO: 16, SEQ ID NO: 17, and SEQ ID NO: 18, respectively, or
heavy chain CDR1 (HCDR1), heavy chain CDR2 (HCDR2), and heavy chain CDR3 (HCDR3), having an amino acid sequence as set forth in SEQ ID NO: 20, SEQ ID NO: 21, and SEQ ID NO: 22, respectively,
wherein each of the HCDR1 or HCDR2 may at most comprise three, two, or one conservative amino acid variations.

23. The bispecific binding moiety according to any of the preceding clauses, wherein the FAP binding domain comprises a heavy chain variable region having an amino acid sequence as set forth in any one of SEQ ID NOs: 11, 15 or 19, or having at least 80%, 85%, 90%, or 95% sequence identity within the framework region thereto.

24. The bispecific binding moiety according to any of the preceding clauses, wherein the FAP binding domain comprises a light chain variable region comprising light chain CDR1 (LCDR1), light chain CDR2 (LCDR2), and light chain CDR3 (LCDR3), having an amino acid sequence as set forth in SEQ ID NO: 53, SEQ ID NO: 54, and SEQ ID NO: 55, respectively, or a variant thereof comprising at most three, two, or one amino acid variations in each LCDR.

25. The bispecific binding moiety according to any of the preceding clauses, wherein the FAP binding domain comprises a light chain variable region having an amino acid sequence as set forth in SEQ ID NO: 52, or having at least 80%, 85%, 90%, or 95% sequence identity thereto.

26. The bispecific binding moiety according to any of the preceding clauses, wherein the TGF-βRII binding domain comprises a heavy chain variable region comprising:
heavy chain CDR1 (HCDR1), heavy chain CDR2 (HCDR2), and heavy chain CDR3 (HCDR3), having an amino acid sequence as set forth in SEQ ID NO: 24, SEQ ID NO: 25, and SEQ ID NO: 26, respectively,
heavy chain CDR1 (HCDR1), heavy chain CDR2 (HCDR2), and heavy chain CDR3 (HCDR3), having an amino acid sequence as set forth in SEQ ID NO: 24, SEQ ID NO: 29, and SEQ ID NO: 30, respectively,
heavy chain CDR1 (HCDR1), heavy chain CDR2 (HCDR2), and heavy chain CDR3 (HCDR3), having an amino acid sequence as set forth in SEQ ID NO: 24, SEQ ID NO: 33, and SEQ ID NO: 34, respectively, or
heavy chain CDR1 (HCDR1), heavy chain CDR2 (HCDR2), and heavy chain CDR3 (HCDR3), having an amino acid sequence as set forth in SEQ ID NO: 24, SEQ ID NO: 37, and SEQ ID NO: 34, respectively;
wherein each of the HCDR1 or HCDR2 may comprise at most three, two, or one conservative amino acid variations.

27. The bispecific binding moiety according to any of the preceding clauses, wherein the TGF-βRII binding domain comprises a heavy chain variable region having an amino acid sequence as set forth in any one of SEQ ID NOs: 23, 27, 31 or 35, or having at least 80%, 85%, 90%, or 95% sequence identity within the framework region thereto.

28. The bispecific binding moiety according to any of the preceding clauses, wherein the TGF-βRII binding domain comprises a light chain variable region comprising light chain CDR1 (LCDR1), light chain CDR2 (LCDR2), and light chain CDR3 (LCDR3), having an amino acid sequence as set forth in SEQ ID NO: 53, SEQ ID NO: 54, and SEQ ID NO: 55, respectively, or a variant thereof comprising at most three, two, or one amino acid variations in each LCDR.

29. The bispecific binding moiety according to any of the preceding clauses, wherein the TGF-βRII binding domain comprises a light chain variable region having an amino acid sequence as set forth in SEQ ID NO: 52, or having at least 80%, 85%, 90%, or 95% sequence identity thereto.

30. A bispecific binding moiety according to any of the preceding clauses, wherein the TGF-βRII binding domain comprises a heavy chain variable region comprising:
heavy chain CDR1 (HCDR1), heavy chain CDR2 (HCDR2), and heavy chain CDR3 (HCDR3), having an amino acid sequence as set forth in SEQ ID NO: 24, SEQ ID NO: 25, and SEQ ID NO: 26, respectively,
heavy chain CDR1 (HCDR1), heavy chain CDR2 (HCDR2), and heavy chain CDR3 (HCDR3), having an amino acid sequence as set forth in SEQ ID NO: 24, SEQ ID NO: 29, and SEQ ID NO: 30, respectively,
heavy chain CDR1 (HCDR1), heavy chain CDR2 (HCDR2), and heavy chain CDR3 (HCDR3), having an amino acid sequence as set forth in SEQ ID NO: 24, SEQ ID NO: 33, and SEQ ID NO: 34, respectively, or
heavy chain CDR1 (HCDR1), heavy chain CDR2 (HCDR2), and heavy chain CDR3 (HCDR3), having an amino acid sequence as set forth in SEQ ID NO: 24, SEQ ID NO: 37, and SEQ ID NO: 34, respectively;
wherein each of the HCDR1 or HCDR2 may comprise at most three, two, or one conservative amino acid variations.

31. The bispecific binding moiety according to any of the preceding clauses, wherein the TGF-βRII binding domain comprises a heavy chain variable region having an amino acid sequence as set forth in any one of SEQ ID NOs: 23, 27, 31 or 35, or having at least 80%, 85%, 90%, or 95% sequence identity within the framework region thereto.

32. The bispecific binding moiety according to any of the preceding clauses, wherein the TGF-βRII binding domain comprises a light chain variable region comprising light chain CDR1 (LCDR1), light chain CDR2 (LCDR2), and light chain CDR3 (LCDR3), having an amino acid sequence as set forth in SEQ ID NO: 53, SEQ ID NO: 54, and SEQ ID NO: 55, respectively, or a variant thereof comprising at most three, two, or one amino acid variations in each LCDR.

33. The bispecific binding moiety according to any of the preceding clauses, wherein the TGF-βRII binding domain comprises a light chain variable region having an amino acid sequence as set forth in SEQ ID NO: 52, or having at least 80%, 85%, 90%, or 95% sequence identity thereto.

34. The bispecific binding moiety according to any of the preceding clauses, wherein the FAP binding domain comprises a heavy chain variable region comprising:
heavy chain CDR1 (HCDR1), heavy chain CDR2 (HCDR2), and heavy chain CDR3 (HCDR3), having an amino acid sequence as set forth in SEQ ID NO: 12, SEQ ID NO: 13, and SEQ ID NO: 14, respectively,
heavy chain CDR1 (HCDR1), heavy chain CDR2 (HCDR2), and heavy chain CDR3 (HCDR3), having an amino acid sequence as set forth in SEQ ID NO: 16, SEQ ID NO: 17, and SEQ ID NO: 18, respectively, or
heavy chain CDR1 (HCDR1), heavy chain CDR2 (HCDR2), and heavy chain CDR3 (HCDR3), having an amino acid sequence as set forth in SEQ ID NO: 20, SEQ ID NO: 21, and SEQ ID NO: 22, respectively,
wherein each of the HCDRs may comprise at most three, two, or one conservative amino acid variations.

35. The bispecific binding moiety according to any of the preceding clauses, wherein the FAP binding domain comprises a heavy chain variable region having an amino acid sequence as set forth in any one of SEQ ID NOs: 11, 15 or 19, or having at least 80%, 85%, 90%, or 95% sequence identity within the framework region thereto.

36. The bispecific binding moiety according to any of the preceding clauses, wherein the FAP binding domain comprises a light chain variable region comprising light chain CDR1 (LCDR1), light chain CDR2 (LCDR2), and light chain CDR3 (LCDR3), having an amino acid sequence as set forth in SEQ ID NO: 53, SEQ ID NO: 54, and SEQ ID NO: 55, respectively, or a variant thereof comprising at most three, two, or one amino acid variations in each LCDR.

37. The bispecific binding moiety according to any of the preceding clauses, wherein the FAP binding domain comprises a light chain variable region having an amino acid sequence as set forth in SEQ ID NO: 52, or having at least 80%, 85%, 90%, or 95% sequence identity thereto.

38. The bispecific binding moiety according to any of the preceding clauses, wherein the bispecific binding moiety comprises a Fab domain that binds FAP, a Fab domain that binds TGF-βRII and an Fc region.

39. The bispecific binding moiety according to any of the preceding clauses, wherein the Fc region has enhanced or reduced immune effector function.

40. The bispecific binding moiety according to any of the preceding clauses, wherein the Fc region has enhanced immune cell effector function, in particular enhanced ADCC activity.

41. The bispecific binding moiety according to any of the preceding clauses, wherein the bispecific binding moiety is afucosylated.

42. The bispecific binding moiety according to any of the preceding clauses, wherein the Fc region has reduced immune cell effector function, in particular reduced ADCC and/or ADCP activity.

43. The bispecific binding moiety according to any of the preceding clauses, wherein the Fc region has L235G and/or G236R mutations in the CH2 domain (EU numbering).

44. The bispecific binding moiety according to any of the preceding clauses, wherein the bispecific binding moiety is a bispecific antibody.

45. A pharmaceutical composition comprising an effective amount of a bispecific binding moiety according to any of the preceding clauses, and a pharmaceutically acceptable carrier.

46. The bispecific binding moiety or the pharmaceutical composition according to any of the preceding clauses, for use in therapy.

47. The bispecific binding moiety or the pharmaceutical composition according to any of the preceding clauses, for use in the treatment of cancer.

48. The bispecific binding moiety according to any of the preceding clauses, and a second binding moiety that binds PD-1, for use in therapy.

49. The bispecific binding moiety according to any of the preceding clauses, and a second binding moiety that binds PD-1, for use in the treatment of cancer.

50. The bispecific binding moiety according to any of the preceding clauses for use in therapy, wherein the therapy further comprises administering a PD-1 inhibitor.

51. The bispecific binding moiety according to any of the preceding clauses for use in the treatment of cancer, wherein the treatment further comprises administering a PD-1 inhibitor.

52. The bispecific binding moiety according to any of the preceding clauses, wherein the second binding moiety that binds PD-1 is pembrolizumab.

53. A method for treating a disease in a subject, comprising administering a therapeutically effective amount of a bispecific binding moiety or a pharmaceutical composition according to any one of the preceding clauses, to the subject in need thereof.

54. A method for treating cancer in a subject, comprising administering a therapeutically effective amount of a bispecific binding moiety or a pharmaceutical composition according to any one of the preceding clauses, to the subject in need thereof.

55. The method of treatment according to any of the preceding clauses, wherein the method further comprises administering an effective amount of a second binding moiety that binds PD-1.

56. The method of treatment according to any of the preceding clauses, wherein the second binding moiety that binds PD-1 is pembrolizumab.

57. A nucleic acid sequence encoding a heavy chain variable region as defined in any one of the preceding clauses.

58. A vector comprising a nucleic acid sequence as defined in any one of the preceding clauses.

59. The vector according to any one of the preceding clauses, wherein the vector further comprises a nucleic acid sequence encoding a CH1 region and preferably a hinge, CH2 and CH3 region.

60. The vector according to any one of the preceding clauses, wherein the vector further comprises at least one nucleic acid sequence encoding a light chain variable region, and preferably a CL region.

61. The vector according to any one of the preceding clauses, wherein the light chain variable region is a light chain variable region of a light chain that is capable of pairing with multiple heavy chains having different epitope specificities.

62. A cell comprising one or more nucleic acids that encode the heavy chain variable region of a FAP binding domain as defined in any one of the preceding clauses and the heavy chain variable region of a TGF-βRII binding domain as defined in any one of the preceding clauses.

63. The cell according to any of the preceding clauses, wherein the one or more nucleic acids further encode a CH1 region and preferably a hinge, CH2 and CH3 region.

64. The cell according to any of the preceding clauses, wherein the one or more nucleic acids further encode a light chain variable region, in particular a light chain variable region as defined in any one of the preceding clauses, and preferably a CL region.

65. A cell producing a bispecific binding moiety as defined in any one of the preceding clauses.

66. A bispecific binding moiety that competes with a bispecific binding moiety as defined in any of the preceding clauses, for binding to FAP and TGF-βRII.

67. A polypeptide selected from:
a polypeptide comprising a heavy chain CDR1 (HCDR1) having an amino acid sequence as set forth in SEQ ID NO: 16, a heavy chain CDR2 (HCDR2) having an amino acid sequence as set forth in SEQ ID NO: 17, and a heavy chain CDR3 (HCDR3) having an amino acid sequence as set forth in SEQ ID NO: 18;
a polypeptide comprising a heavy chain CDR1 (HCDR1) having an amino acid sequence as set forth in SEQ ID NO: 20, a heavy chain CDR2 (HCDR2) having an amino acid sequence as set forth in SEQ ID NO: 21, and a heavy chain CDR3 (HCDR3) having an amino acid sequence as set forth in SEQ ID NO: 22;
a polypeptide comprising a heavy chain CDR1 (HCDR1) having an amino acid sequence as set forth in SEQ ID NO: 12, a heavy chain CDR2 (HCDR2) having an amino acid sequence as set forth in SEQ ID NO: 13, and a heavy chain CDR3 (HCDR3) having an amino acid sequence as set forth in SEQ ID NO: 14; or
a polypeptide comprising a heavy chain CDR1 (HCDR1) having an amino acid sequence as set forth in SEQ ID NO: 70, a heavy chain CDR2 (HCDR2) having an amino acid sequence as set forth in SEQ ID NO: 71, and a heavy chain CDR3 (HCDR3) having an amino acid sequence as set forth in SEQ ID NO: 72.

68. The polypeptide according to clause 67, wherein the polypeptide comprises a heavy chain variable region having an amino acid sequence as set forth in SEQ ID NO: 15; 19; 11; 69, or having at least 80%, or at least 85%, or at least 90%, or at least 95%, sequence identity thereto.

69. The polypeptide according to clause 67 or 68, further comprising a CH1 region.

70. A FAP binding domain comprising the polypeptide according to any one of clauses 67-69.

71. The FAP binding domain according to clause 70, wherein the FAP binding domain further comprises a polypeptide comprising light chain CDR1 (LCDR1), light chain CDR2 (LCDR2), and light chain CDR3 (LCDR3), having an amino acid sequence as set forth in SEQ ID NO: 53, SEQ ID NO: 54, and SEQ ID NO: 55, respectively.

72. The FAP binding domain according to clause 70 or 71, wherein the FAP binding domain comprises a light chain variable region having an amino acid sequence as set forth in SEQ ID NO: 52, or having at least 80%, or at least 85%, or at least 90%, or at least 95%, sequence identity thereto.

73. The FAP binding domain according to any one of clauses 70-72, further comprising a CL region.

74. A FAP binding domain that binds to human FAP and mouse FAP.

75. The FAP binding domain according to clause 74, wherein the FAP binding domain comprises a polypeptide comprising a heavy chain CDR1 (HCDR1) having an amino acid sequence as set forth in SEQ ID NO: 70, a heavy chain CDR2 (HCDR2) having an amino acid sequence as set forth in SEQ ID NO: 71, and a heavy chain CDR3 (HCDR3) having an amino acid sequence as set forth in SEQ ID NO: 72.

76. The FAP binding domain according to clause 75, wherein the polypeptide comprises a heavy chain variable region having an amino acid sequence as set forth in SEQ ID NO: 69, or having at least 80%, or at least 85%, or at least 90%, or at least 95%, sequence identity thereto.

77. The FAP binding domain according to clause 75 or 76, wherein the polypeptide further comprises a CH1 region.

78. The FAP binding domain according to any one of clauses 75-77, wherein the FAP binding domain further comprises a polypeptide comprising light chain CDR1 (LCDR1), light chain CDR2 (LCDR2), and light chain CDR3 (LCDR3), having an amino acid sequence as set forth in SEQ ID NO: 53, SEQ ID NO: 54, and SEQ ID NO: 55, respectively, or a variant thereof.

79. The FAP binding domain according to clause 78, wherein the FAP binding domain comprises a light chain variable region having an amino acid sequence as set forth in SEQ ID NO: 52, or having at least 80%, or at least 85%, or at least 90%, or at least 95%, sequence identity thereto.

80. The FAP binding domain according to any one of clauses 75-79, further comprising a CL region.

81. A binding moiety comprising a polypeptide as defined in any one of clauses 67-69, or a FAP binding domain as defined in any one of clauses 70-80.

82. The binding moiety according to clause 81, wherein the binding moiety is a monospecific binding moiety, in particular a bivalent monospecific antibody.

83. A pharmaceutical composition comprising an effective amount of a polypeptide as defined in any one of clauses 67-69, or a FAP binding domain as defined in any one of clauses 70-80, or a binding moiety as defined in clause 81 or 82, and a pharmaceutically acceptable carrier.

84. A polypeptide as defined in any one of clauses 67-69, or a FAP binding domain as defined in any one of clauses 70-80, or a binding moiety as defined in clause 81 or 82, or a pharmaceutical composition as defined in clause 83, for use in therapy.

85. A polypeptide as defined in any one of clauses 67-69, or a FAP binding domain as defined in any one of clauses 70-80, or a binding moiety as defined in clause 81 or 82, or a pharmaceutical composition as defined in clause 83, for use in the treatment of cancer.

86. A method for treating a disease, comprising administering an effective amount of a polypeptide as defined in any one of clauses 67-69, or a FAP binding domain as defined in any one of clauses 70-80, or a binding moiety as defined in clause 81 or 82, or a pharmaceutical composition as defined in clause 83, to an individual in need thereof.

87. A method for treating cancer, comprising administering an effective amount of a polypeptide as defined in any one of clauses 67-69, or a FAP binding domain as defined in any one of clauses 70-80, or a binding moiety as defined in clause 81 or 82, or a pharmaceutical composition as defined in clause 83, to an individual in need thereof.

88. A nucleic acid comprising a sequence encoding the polypeptide as defined in any one of clauses 67-69.

89. A vector comprising a nucleic acid as defined in clause 88.

90. The vector according to clause 89, wherein the vector further comprises a nucleic acid sequence encoding a CH1 region and preferably a hinge, CH2 and CH3 region.

91. The vector according to clause 89 or 90, wherein the vector further comprises at least one nucleic acid sequence encoding a light chain variable region, and preferably a CL region.

92. The vector according to clause 91, wherein the light chain variable region is a light chain variable region comprising the light chain CDRs as defined in clause 78 or is the light chain variable region as defined in clause 79.

93. A cell comprising a nucleic acid as defined in clause 88, or a vector as defined in any one of the clauses 89-92.

94. The cell according to clause 93, wherein the cell further comprises a nucleic acid comprising a sequence that encodes a CH1 region and preferably a hinge, CH2 and CH3 region.

95. The cell according to clause 93 or 94, wherein the cell further comprises at least one nucleic acid comprising a sequence that encodes a light chain variable region, and preferably a CL region.

96. A cell producing a polypeptide as defined in any one of clauses 67-69, or a FAP binding domain as defined in any one of clauses 70-80, or a binding moiety as defined in clause 81 or 82.

97. The cell according to clause 96, wherein the cell is a recombinant cell comprising the vector as defined in any one of clauses 89-92.

98. The bispecific binding moiety according to any of the preceding clauses, wherein the Fc region has immune cell effector function, in particular ADCP activity.

1. A bispecific binding moiety comprising a FAP binding domain and a TGF-βRII binding domain, wherein the TGF-βRII binding domain blocks TGF-βRII binding to TGF-βRII ligand.

2. The bispecific binding moiety according to claim 1, wherein the bispecific binding moiety blocks TGF-βRII mediated signaling in a cell expressing FAP and TGF-βRII.

3. The bispecific binding moiety according to claim 2, wherein the potency of the bispecific binding moiety in blocking TGF-βRII mediated signaling is 2.0-500 fold higher than the potency of a reference anti-TGF-βRII antibody in a cell expressing FAP and TGF-βRII, wherein the reference anti-TGF-βRII antibody is a bivalent monospecific antibody comprising a heavy chain having an amino acid sequence as set forth in SEQ ID NO: 1 and a light chain having an amino acid sequence as set forth in SEQ ID NO: 2.

4. The bispecific binding moiety according to claim 2, wherein the cell expressing FAP and TGF-βRII is a fibroblast.

5. The bispecific binding moiety according to claim 2, wherein the cell expressing FAP and TGF-βRII is a primary cancer associated fibroblast (CAF).

6. The bispecific binding moiety according to claim 2, wherein the blocking of TGF-βRII mediated signaling is measured as reduction in pSMAD2 expression in a TGF-βRII signaling inhibition assay.

7. The bispecific binding moiety according to claim 1, wherein the bispecific binding moiety has a higher potency in blocking TGF-βRII mediated signaling in a cell expressing FAP and TGF-βRII than in a cell expressing TGF-βRII and no, or undetectable levels of FAP, wherein the cell expressing FAP and TGF-βRII is a A549-FAP⁺ cell and wherein the cell expressing TGF-βRII and no, or undetectable levels of FAP is a A549 parental cell.

8. The bispecific binding moiety according to claim 7, wherein the potency in blocking TGF-βRII mediated signaling is measured as a reduction in pSMAD2 levels in a mixed culture pSMAD2 assay.

9. The bispecific binding moiety according to claim 7, wherein the potency of the bispecific binding moiety in blocking TGF-βRII mediated signaling in a cell expressing FAP and TGF-βRII is between about 100-20,000 fold higher than in a cell expressing TGF-βRII and no, or undetectable levels of FAP.

10. A bispecific binding moiety comprising a FAP binding domain and a TGF-βRII binding domain, wherein the FAP binding domain binds to FAP expressed on a first cell and the TGF-βRII binding domain binds to TGF-βRII expressed on a second cell.

11. The bispecific binding moiety according to claim 10, wherein upon binding of the FAP binding domain to FAP expressed on the first cell and binding of the TGF-βRII binding domain to TGF-βRII expressed on the second cell, the TGF-βRII binding domain blocks TGF-βRII mediated signaling in the second cell.

12. The bispecific binding moiety according to claim 10, wherein the first cell is a fibroblast cell.

13. The bispecific binding moiety according to claim 10, wherein the second cell is a non-fibroblast cell.

14. The bispecific binding moiety according to claim 13, wherein the second cell is an immune effector cell or a tumor cell.

15. The bispecific binding moiety according to claim 14, wherein the second cell is a T-cell.

16. The bispecific binding moiety according to claim 11, wherein the blocking of TGF-βRII mediated signaling in the second cell is measured in a TGF-β reporter assay.

17. The bispecific binding moiety according to claim 16, wherein the first cell used in the TGF-β reporter assay is a FAP expressing MRC5 cell and the second cell is a TGF-βRII expressing HEK-Blue cell.

18. A bispecific binding moiety comprising a FAP binding domain and a TGF-βRII binding domain, wherein the FAP binding domain comprises a heavy chain variable region comprising:
heavy chain CDR1 (HCDR1), heavy chain CDR2 (HCDR2), and heavy chain CDR3 (HCDR3), having an amino acid sequence as set forth in SEQ ID NO: 12, SEQ ID NO: 13, and SEQ ID NO: 14, respectively,
heavy chain CDR1 (HCDR1), heavy chain CDR2 (HCDR2), and heavy chain CDR3 (HCDR3), having an amino acid sequence as set forth in SEQ ID NO: 16, SEQ ID NO: 17, and SEQ ID NO: 18, respectively, or
heavy chain CDR1 (HCDR1), heavy chain CDR2 (HCDR2), and heavy chain CDR3 (HCDR3), having an amino acid sequence as set forth in SEQ ID NO: 20, SEQ ID NO: 21, and SEQ ID NO: 22, respectively,
wherein each of the HCDR1 or HCDR2 may comprise at most three, two, or one amino acid variations.

19. The bispecific binding moiety according to claim 18, wherein the FAP binding domain comprises a heavy chain variable region having an amino acid sequence as set forth in any one of SEQ ID NOs: 11, 15 or 19, or having at least 80%, 85%, 90%, or 95% sequence identity thereto.

20. The bispecific binding moiety according to claim 18, wherein the FAP binding domain comprises a light chain variable region comprising light chain CDR1 (LCDR1), light chain CDR2 (LCDR2), and light chain CDR3 (LCDR3), having an amino acid sequence as set forth in SEQ ID NO: 53, SEQ ID NO: 54, and SEQ ID NO: 55, respectively, or a variant thereof comprising at most three, two, or one amino acid variations in each LCDR.

21. The bispecific binding moiety according to claim 18, wherein the FAP binding domain comprises a light chain variable region having an amino acid sequence as set forth in SEQ ID NO: 52, or having at least 80%, 85%, 90%, or 95% sequence identity thereto.

22. The bispecific binding moiety according to claim 18, wherein the TGF-βRII binding domain comprises a heavy chain variable region comprising:
heavy chain CDR1 (HCDR1), heavy chain CDR2 (HCDR2), and heavy chain CDR3 (HCDR3), having an amino acid sequence as set forth in SEQ ID NO: 24, SEQ ID NO: 25, and SEQ ID NO: 26, respectively,
heavy chain CDR1 (HCDR1), heavy chain CDR2 (HCDR2), and heavy chain CDR3 (HCDR3), having an amino acid sequence as set forth in SEQ ID NO: 24, SEQ ID NO: 29, and SEQ ID NO: 30, respectively,
heavy chain CDR1 (HCDR1), heavy chain CDR2 (HCDR2), and heavy chain CDR3 (HCDR3), having an amino acid sequence as set forth in SEQ ID NO: 24, SEQ ID NO: 33, and SEQ ID NO: 34, respectively, or
heavy chain CDR1 (HCDR1), heavy chain CDR2 (HCDR2), and heavy chain CDR3 (HCDR3), having an amino acid sequence as set forth in SEQ ID NO: 24, SEQ ID NO: 37, and SEQ ID NO: 34, respectively;
wherein each of the HCDR1 or HCDR2 may comprise at most three, two, or one amino acid variations.

23. The bispecific binding moiety according to claim 18, wherein the TGF-βRII binding domain comprises a heavy chain variable region having an amino acid sequence as set forth in any one of SEQ ID NOs: 23, 27, 31 or 35, or having at least 80%, 85%, 90%, or 95% sequence identity thereto.

24. The bispecific binding moiety according to claim 18, wherein the TGF-βRII binding domain comprises a light chain variable region comprising light chain CDR1 (LCDR1), light chain CDR2 (LCDR2), and light chain CDR3 (LCDR3), having an amino acid sequence as set forth in SEQ ID NO: 53, SEQ ID NO: 54, and SEQ ID NO: 55, respectively, or a variant thereof comprising at most three, two, or one amino acid variations in each LCDR.

25. The bispecific binding moiety according to claim 18, wherein the TGF-βRII binding domain comprises a light chain variable region having an amino acid sequence as set forth in SEQ ID NO: 52, or having at least 80%, 85%, 90%, or 95% sequence identity thereto.

26. A bispecific binding moiety, comprising a FAP binding domain and a TGF-βRII binding domain, wherein the TGF-βRII binding domain comprises a heavy chain variable region comprising:
heavy chain CDR1 (HCDR1), heavy chain CDR2 (HCDR2), and heavy chain CDR3 (HCDR3), having an amino acid sequence as set forth in SEQ ID NO: 24, SEQ ID NO: 25, and SEQ ID NO: 26, respectively,
heavy chain CDR1 (HCDR1), heavy chain CDR2 (HCDR2), and heavy chain CDR3 (HCDR3), having an amino acid sequence as set forth in SEQ ID NO: 24, SEQ ID NO: 29, and SEQ ID NO: 30, respectively,
heavy chain CDR1 (HCDR1), heavy chain CDR2 (HCDR2), and heavy chain CDR3 (HCDR3), having an amino acid sequence as set forth in SEQ ID NO: 24, SEQ ID NO: 33, and SEQ ID NO: 34, respectively, or
heavy chain CDR1 (HCDR1), heavy chain CDR2 (HCDR2), and heavy chain CDR3 (HCDR3), having an amino acid sequence as set forth in SEQ ID NO: 24, SEQ ID NO: 37, and SEQ ID NO: 34, respectively;
wherein each of the HCDR1 or HCDR2 may comprise at most three, two, or one amino acid variations.

27. The bispecific binding moiety according to claim 26, wherein the TGF-βRII binding domain comprises a heavy chain variable region having an amino acid sequence as set forth in any one of SEQ ID NOs: 23, 27, 31 or 35, or having at least 80%, 85%, 90%, or 95% sequence identity thereto.

28. The bispecific binding moiety according to claim 26, wherein the TGF-βRII binding domain comprises a light chain variable region comprising light chain CDR1 (LCDR1), light chain CDR2 (LCDR2), and light chain CDR3 (LCDR3), having an amino acid sequence as set forth in SEQ ID NO: 53, SEQ ID NO: 54, and SEQ ID NO: 55, respectively, or a variant thereof comprising at most three, two, or one amino acid variations in each LCDR.

29. The bispecific binding moiety according to claim 26, wherein the TGF-βRII binding domain comprises a light chain variable region having an amino acid sequence as set forth in SEQ ID NO: 52, or having at least 80%, 85%, 90%, or 95% sequence identity thereto.

30. The bispecific binding moiety according to claim 26, wherein the FAP binding domain comprises a heavy chain variable region comprising:
heavy chain CDR1 (HCDR1), heavy chain CDR2 (HCDR2), and heavy chain CDR3 (HCDR3), having an amino acid sequence as set forth in SEQ ID NO: 12, SEQ ID NO: 13, and SEQ ID NO: 14, respectively,
heavy chain CDR1 (HCDR1), heavy chain CDR2 (HCDR2), and heavy chain CDR3 (HCDR3), having an amino acid sequence as set forth in SEQ ID NO: 16, SEQ ID NO: 17, and SEQ ID NO: 18, respectively, or
heavy chain CDR1 (HCDR1), heavy chain CDR2 (HCDR2), and heavy chain CDR3 (HCDR3), having an amino acid sequence as set forth in SEQ ID NO: 20, SEQ ID NO: 21, and SEQ ID NO: 22, respectively,
wherein each of the HCDR1 or HCDR2 may comprise at most three, two, or one amino acid variations.

31. The bispecific binding moiety according to claim 26, wherein the FAP binding domain comprises a heavy chain variable region having an amino acid sequence as set forth in any one of SEQ ID NOs: 11, 15 or 19, or having at least 80%, 85%, 90%, or 95% sequence identity thereto.

32. The bispecific binding moiety according to claim 26, wherein the FAP binding domain comprises a light chain variable region comprising light chain CDR1 (LCDR1), light chain CDR2 (LCDR2), and light chain CDR3 (LCDR3), having an amino acid sequence as set forth in SEQ ID NO: 53, SEQ ID NO: 54, and SEQ ID NO: 55, respectively, or a variant thereof comprising at most three, two, or one amino acid variations in each LCDR.

33. The bispecific binding moiety according to claim 26, wherein the FAP binding domain comprises a light chain variable region having an amino acid sequence as set forth in SEQ ID NO: 52, or having at least 80%, 85%, 90%, or 95% sequence identity

34. The bispecific binding moiety according to claim 1, wherein the bispecific binding moiety comprises a Fab domain that binds FAP, a Fab domain that binds TGF-βRII and an Fc region.

35. The bispecific binding moiety according to claim 34, wherein the Fc region has enhanced or reduced immune effector function.

36. The bispecific binding moiety according to claim 34, wherein the Fc region has enhanced immune cell effector function, in particular enhanced ADCC activity.

37. The bispecific binding moiety according to claim 36, wherein the bispecific binding moiety is afucosylated.

38. The bispecific binding moiety according to claim 34, wherein the Fc region has reduced immune cell effector function, in particular reduced ADCC and/or ADCP activity.

39. The bispecific binding moiety according to claim 38, wherein the Fc region has L235G and/or G236R mutations in the CH2 domain (EU numbering).

40. A pharmaceutical composition comprising an effective amount of a bispecific binding moiety according to claim 1, and a pharmaceutically acceptable carrier.

41. A bispecific binding moiety according to claim 1, for use in therapy.

42. A bispecific binding moiety according to claim 1, for use in the treatment of cancer.

43. A combination of a bispecific binding moiety according to claim 1 and a second binding moiety that binds PD-1, for use in therapy.

44. A combination of a bispecific binding moiety according to claim 1 and a second binding moiety that binds PD-1 for use in the treatment of cancer.

45. The combination according to claim 43, wherein the second binding moiety that binds PD-1 is pembrolizumab.

46. A method for treating a disease in a subject, comprising administering a therapeutically effective amount of a bispecific binding moiety according to claim 1.

47. A method for treating cancer in a subject, comprising administering a therapeutically effective amount of a bispecific binding moiety according to claim 1.

48. The method of treatment according to claim 46, wherein the method further comprises administering an effective amount of a second binding moiety that binds PD-1.

49. The method of treatment according to claim 48, wherein the second binding moiety that binds PD-1 is pembrolizumab.

50. A nucleic acid sequence encoding a heavy chain variable region as defined in claim 18.

51. A vector comprising a nucleic acid sequence as claimed in claim 50.

52. The vector according to claim 51, wherein the vector further comprises a nucleic acid sequence encoding a CH1 region and preferably a hinge, CH2 and CH3 region.

53. The vector according to claim 52, wherein the vector further comprises at least one nucleic acid sequence encoding a light chain variable region according to claim 20 and preferably a CL region.

54. The vector according to claim 53, wherein the light chain variable region is a light chain variable region of a light chain that is capable of pairing with multiple heavy chains having different epitope specificities.

55. A cell comprising one or more nucleic acids that encode the heavy chain variable region of a FAP binding domain of claim 18 and a heavy chain variable region of a TGF-βRII binding domain.

56. The cell according to claim 55, wherein the one or more nucleic acids further encode a CH1 region and preferably a hinge, CH2 and CH3 region.

57. The cell according to claim 55, wherein the one or more nucleic acids further encode a light chain variable region, in particular a light chain variable region of claim 20 and preferably a CL region.

58. A cell producing a bispecific binding moiety as claimed in claim 1.

59. A bispecific binding moiety that competes with a bispecific binding moiety as claimed in claim 1 for binding to FAP and TGF-βRII.

60. A polypeptide selected from:
a polypeptide comprising a heavy chain CDR1 (HCDR1) having an amino acid sequence as set forth in SEQ ID NO: 16, a heavy chain CDR2 (HCDR2) having an amino acid sequence as set forth in SEQ ID NO: 17, and a heavy chain CDR3 (HCDR3) having an amino acid sequence as set forth in SEQ ID NO: 18;
a polypeptide comprising a heavy chain CDR1 (HCDR1) having an amino acid sequence as set forth in SEQ ID NO: 20, a heavy chain CDR2 (HCDR2) having an amino acid sequence as set forth in SEQ ID NO: 21, and a heavy chain CDR3 (HCDR3) having an amino acid sequence as set forth in SEQ ID NO: 22;
a polypeptide comprising a heavy chain CDR1 (HCDR1) having an amino acid sequence as set forth in SEQ ID NO: 12, a heavy chain CDR2 (HCDR2) having an amino acid sequence as set forth in SEQ ID NO: 13, and a heavy chain CDR3 (HCDR3) having an amino acid sequence as set forth in SEQ ID NO: 14;
a polypeptide comprising a heavy chain CDR1 (HCDR1) having an amino acid sequence as set forth in SEQ ID NO: 70, a heavy chain CDR2 (HCDR2) having an amino acid sequence as set forth in SEQ ID NO: 71, and a heavy chain CDR3 (HCDR3) having an amino acid sequence as set forth in SEQ ID NO: 72.

61. The polypeptide according to claim 60, wherein the polypeptide comprises a heavy chain variable region having an amino acid sequence as set forth in SEQ ID NO: 15; 19; 11; 69, or having at least 80%, or at least 85%, or at least 90%, or at least 95%, sequence identity thereto.

62. A FAP binding domain comprising the polypeptide according to claim 60.

63. A FAP binding domain that binds to human FAP and mouse FAP.

64. The FAP binding domain according to claim 63, wherein the FAP binding domain comprises a polypeptide comprising a heavy chain CDR1 (HCDR1) having an amino acid sequence as set forth in SEQ ID NO: 70, a heavy chain CDR2 (HCDR2) having an amino acid sequence as set forth in SEQ ID NO: 71, and a heavy chain CDR3 (HCDR3) having an amino acid sequence as set forth in SEQ ID NO: 72.

65. The FAP binding domain according to claim 64, wherein the polypeptide comprises a heavy chain variable region having an amino acid sequence as set forth in SEQ ID NO: 69, or having at least 80%, or at least 85%, or at least 90%, or at least 95%, sequence identity thereto.

66. A binding moiety comprising a polypeptide as claimed in claim 60.

67. A pharmaceutical composition comprising an effective amount of a polypeptide as claimed in claim 60 and a pharmaceutically acceptable carrier.

68. A polypeptide as claimed in claim 60, for use in therapy.

69. A polypeptide as claimed in claim 60 for use in the treatment of cancer.

70. A method for treating a disease, comprising administering an effective amount of a polypeptide as claimed in claim 60 to an individual in need thereof.

71. A method for treating cancer, comprising administering an effective amount of a polypeptide as claimed in claim 60 to an individual in need thereof.

72. A nucleic acid comprising a sequence encoding the polypeptide as claimed in claim 60.

73. A vector comprising a nucleic acid as claimed in claim 72.

74. A cell comprising a nucleic acid as claimed in claim 72.

75. A cell producing a polypeptide as claimed in claim 60.
